(19) United States
(12) Patent Application Publication
(10) Pub. No.: US 2025/0223326 A1
(43) Pub. Date: Jul. 10, 2025

(54) SYNCITIN-1 FUSION PROTEINS AND USES THEREOF FOR CARGO DELIVERY INTO TARGET CELLS

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); FONDATION IMAGINE, Paris (FR); UNIVERSITÉ PARIS CITÉ, Paris (FR)

(72) Inventors: Annarita MICCIO, Paris (FR); Mathieu VON JOEST, Paris (FR)

(21) Appl. No.: 18/853,878

(22) PCT Filed: May 10, 2023
(86) PCT No.: PCT/EP2023/062497

§ 371 Date: Oct. 3, 2024

(30) Foreign Application Priority Data
May 10, 2022 (EP) 22305688.8

(51) Int. Cl. C07K 14/47; C07K 16/26; C07K 16/28; C07K 16/40; C12N 7/00; C12N 15/62

(52) U.S. Cl. CPC C07K 14/4702; C07K 16/26; C07K 16/2812; C07K 16/2815; C07K 16/2896; C07K 16/40; C12N 7/00; C12N 15/625; C07K 2317/622; C07K 2319/02; C07K 2319/33; C12N 2740/16022; C12N 2740/16023; C12N 2740/16042; C12N 2740/16051; C12N 2740/16211

(57) ABSTRACT

The inventors developed a new system to modify tropism of syncytins, envelope proteins, which can be used to functionalize particles such as virus particle or more particularly viral-like particles (VLPs), and used for gene transfer or other applications. In particular, they created a fusion protein containing the Syncytin-1 (SYN) signal sequence (SS), a targeting moiety (either natural or engineered), the SYN protein and a flexible linker between SYN and the targeting moiety to enhance transduction of the cell type expressing the receptor or the antigen targeted by the targeting moiety, for instance hematopoietic stem progenitor cells (HSPCs). The inventors demonstrated that the fusion strategy allows modification of syncytin tropism towards different receptors in order to target f the desired cell type. The system is adaptable to other desired antigens to retarget the fusion protein to a specific cell type.

### FIELD OF THE INVENTION

The present invention is in the field of medicine, in particular in the field of cargo delivery into target cells.

### BACKGROUND OF THE INVENTION

Particles are particularly suitable for cargo delivery in to cells. In particular, virus particles can be spontaneously self-assembled by viral structural proteins under appropriate conditions in vitro. In addition, virus particles possess several features including can be rapidly produced in large quantities through existing expression systems, and highly resembling native viruses in terms of conformation and appearance. Furthermore, virus particles, with a diameter of approximately 20 to 150 nm, also have the characteristics of nanometer materials, such as large surface area, surface-accessible amino acids with reactive moieties (e.g., lysine and glutamic acid residues), inerratic spatial structure, and good biocompatibility. Therefore, assembled virus particles have great potential as a delivery system for specifically carrying a variety of cargos. Several results demonstrate the importance of having both the viral structural protein (e.g. capsid) to form the virus particle and a functional fusogenic envelope on the surface of the virus particle for efficient delivery of cargos into cells. In said context, the fusogenic envelope G glycoprotein of the vesicular stomatitis virus (VSV-G) has been extensively used for enhancing the fusogenicity of virus particles. Other fusogenic proteins have been also investigated for improving the delivery of viral particles. In particular, the interest of syncytin glycoproteins that are envelope proteins of the human endogenous retrovirus family W (HERV-W) was explored. For instance, WO/2017/182607 describes methods to transduce immune cells using lentiviral vectors pseudotyped with an ERV syncytin glycoprotein. More recently, virus particles pseudotyped with murine syncytin and incorporating mammalian Gag homologs were engineered to package, secrete, and deliver specific RNAs (Segel M, Lash B, Song J, Ladha A, Liu C C, Jin X, Mekhedov S L, Macrae R K, Koonin E V, Zhang F. Mammalian retrovirus-like protein PEG10 packages its own mRNA and can be pseudotyped for mRNA delivery. Science. 2021 Aug. 20; 373(6557):882-889. doi: 10.11261/science.abg6155. PMID: 34413232; PMCID: PMC8431961). Together, these results demonstrate that syncytin represents a modular platform suited for development as an efficient therapeutic delivery modality. However, there is still a need for improving the targeting of the virus particles to the target cells so as to enhance the therapeutical efficiency and for avoiding any unspecific effects.

### SUMMARY OF THE INVENTION

The present invention is defined by the claims. In particular, the present invention relates to syncitin-1 fusion proteins and uses thereof for cargo delivery into target cells.

### DETAILED DESCRIPTION OF THE INVENTION

### Main Definitions

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “fusion protein” means a protein created by joining two or more polypeptide sequences together. The fusion polypeptides encompassed in this invention include translation products of a chimeric gene construct that joins the nucleic acid sequences encoding a first polypeptide, e.g., an RNA-binding domain, with the nucleic acid sequence encoding a second polypeptide, e.g., an effector domain, to form a single open-reading frame. In other words, a “fusion polypeptide” or “fusion protein” is a recombinant protein of two or more proteins which are joined by a peptide bond or via several peptides. The fusion protein may also comprise a peptide linker between the two domains. Within the fusion protein, the term “operably linked” is intended to indicate that the peptide of the present invention and the heterologous polypeptide are fused in-frame to each other.

As used herein, the term “linker” has its general meaning in the art and refers to an amino acid sequence of a length sufficient to ensure that the proteins form proper secondary and tertiary structures. Typically, linkers are those which allow the compound to adopt a proper conformation. The most suitable linker sequences (1) will adopt a flexible extended conformation, (2) will not exhibit a propensity for developing ordered secondary structure which could interact with the functional domains of fusion proteins, and (3) will have minimal hydrophobic or charged character which could promote interaction with the functional protein domains.

As used herein, the term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some embodiments, the polynucleotide comprises an mRNA. In other aspect, the mRNA is a synthetic mRNA. In some embodiments, the synthetic mRNA comprises at least one unnatural nucleobase. In some embodiments, all nucleobases of a certain class have been replaced with unnatural nucleobases (e.g., all uridines in a polynucleotide disclosed herein can be replaced with an unnatural nucleobase, e.g., 5-methoxyuridine). In some embodiments, the polynucleotide (e.g., a synthetic RNA or a synthetic DNA) comprises only natural nucleobases, i.e., A, C, T and G in the case of a synthetic DNA, or A, C, T, and U in the case of a synthetic RNA.

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “polynucleotide sequence encoding an amino acid sequence” includes all nucleotide sequences that are degenerate versions of each other and that encode the same amino acid sequence. The phrase “polynucleotide sequence that encodes a protein or a RNA” may also include introns to the extent that the nucleotide sequence encoding the protein may in some version contain an intron(s).

As used herein, the expression “derived from” refers to a process whereby a first component (e.g., a first polypeptide), or information from that first component, is used to isolate, derive or make a different second component (e.g., a second polypeptide that is different from the first one).

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99 or 100% of identity with the second amino acid sequence.

As used herein, the term “mutation” has its general meaning in the art and refers to a substitution, deletion or insertion. In particular, the term “substitution” means that a specific amino acid residue at a specific position is removed and another amino acid residue is inserted into the same position. Within the specification, the mutation are references according to the standard mutation nomenclature.

As used herein, the term “syncytin-1” or “SYN” has its general meaning in the art and refers to a protein found in humans and other primates that is encoded by the ERVW-1 gene (endogenous retrovirus group W envelope member 1). Syncytin-1 is a cell-cell fusion protein whose function is best characterized in placental development. The term is also known as Endogenous retrovirus group W member 1, Env-W, Envelope polyprotein gPr73, Enverin, HERV-7q Envelope protein, HERV-W envelope protein, HERV-W 7921.2 provirus ancestral Env polyprotein and Syncytin. An exemplary amino acid sequence for syncytin-1 is represented by SEQ ID NO: 1. The signal peptide ranges from the amino acid residue at position 1 to the amino acid residue at position 20 in SEQ ID NO: 1. The extracellular domain of syncytin-1 ranges from the amino acid residue at position 21 to the amino acid residue at position 443 in SEQ ID NO:1.

SEQ ID NO: 1 >sp|Q9UQF0|SYCY1_HUMAN Syncytin-1 OS = Homo sapiens OX = 9606
GN = ERVW-1 PE = 1 SV = 1 (the signal peptide is indicated in italic)
MALPYHIFLFTVLLPSFTLTAPPPCRCMTSSSPYQEFLWRMQRPGNIDAPSYRSLSKGTP
TFTAHTHMPRNCYHSATLCMHANTHYWTGKMINPSCPGGLGVTVCWTYFTQTGMSDGGGV
QDQAREKHVKEVISQLTRVHGTSSPYKGLDLSKLHETLRTHTRLVSLENTTLTGLHEVSA
QNPTNCWICLPLNFRPYVSIPVPEQWNNFSTEINTTSVLVGPLVSNLEITHTSNLTCVKF
SNTTYTTNSQCIRWVTPPTQIVCLPSGIFFVCGTSAYRCLNGSSESMCFLSELVPPMTIY
TEQDLYSYVISKPRNKRVPILPFVIGAGVLGALGTGIGGITTSTQFYYKLSQELNGDMER
VADSLVTLQDQLNSLAAVVLQNRRALDLLTAERGGTCLFLGEECCYYVNQSGIVTEKVKE
IRDRIQRRAEELRNTGPWGLLSQWMPWILPELGPLAAIILLLLFGPCIFNLLVNFVSSRI
EAVKLQMEPKMQSKTKIYRRPLDRPASPRSDVNDIKGTPPEEISAAQPLLRPNSAGSS

As used herein, the term “ASCT1” refers to the human neutral amino acid transporter A that is encoded by the SLCIA4 gene. Syncytin-1 can bind to ASCT1 (Antony J M, Ellestad K K, Hammond R, Imaizumi K, Mallet F, Warren K G, Power C. The human endogenous retrovirus envelope glycoprotein, syncytin-1, regulates neuroinflammation and its receptor expression in multiple sclerosis: a role for endoplasmic reticulum chaperones in astrocytes. J Immunol. 2007 Jul. 15; 179(2):1210-24. doi: 10.40491/jimmunol.179.2.1210. PMID: 17617614).

As used herein, the term “ASCT2” refers to the neutral amino acid transporter B(0) that is encoded by the SLCIA5 gene. ASCT2 was described as the receptor for syncytin-1 (Blond J L, Lavillette D, Cheynet V, Bouton O, Oriol G, Chapel-Fernandes S, Mandrand B, Mallet F, Cosset F L. An envelope glycoprotein of the human endogenous retrovirus HERV-W is expressed in the human placenta and fuses cells expressing the type D mammalian retrovirus receptor. J Virol. 2000; 74:3321-3329. doi: 10.1128/JVI.74.7.3321-3329.2000.).

As used herein, the term “SYN480” refers to a polypeptide that consists of the amino acid sequence that ranges from the amino acid residue at position 21 to the amino acid residue at position 480 in SEQ ID NO 1.

As used herein, the term “syncitin-1 polypeptide” or “SYN polypeptide” refers to any polypeptide that derives from syncytin-1 and that comprises the SDGGGXXDXXR (SEQ ID NO: 2) conserved motif essential for syncytin-1-hASCT2 interaction (see Cheynet V, Oriol G, Mallet F. Identification of the hASCT2-binding domain of the Env ERVWE1/syncytin-1 fusogenic glycoprotein. Retrovirology. 2006 Jul. 4; 3:41. doi: 10.1186/1742-4690-3-41. PMID: 16820059; PMCID: PMC1524976.). According to the present invention, the syncytin-1 polypeptide is capable of binding to the ASCT1 receptor preferably ASCT2 receptor as determined by any assay well known in the art (see e.g. Cheynet V. et al. supra).

As used herein, the term “particle” refers to a small object that behaves as a whole unit with respect to its transport and properties i.e. a discrete unit of matter, where the atoms or molecules from which it is formed essentially embody the particle.

As used herein, the term “nanoparticle” refers to a particle having a diameter below about 1000 nm (for example, about 500 nm) and more specifically below about 300 nm. In one embodiment, the term “nanoparticle” refers to particles having diameters in the nano size range, which do not cross over into the micron size range.

As used herein, the term “functionalized” is used interchangeably with the terms “attached” and “bound”.

As used herein, the term “virus particle” has its general meaning in the art and refers to the fully or partially assembled capsid of a virus. A viral particle may or may not contain the viral genome. The term thus encompasses virus-like particle (VLP). Virus particle, with a diameter of approximately 20 to 150 nm, also have the characteristics of nanometer materials, such as large surface area, surface-accessible amino acids with reactive moieties (e.g., lysine and glutamic acid residues), inerratic spatial structure, and good biocompatibility. Therefore, virus particles have great potential as a delivery system for specifically carrying a variety of cargos.

As used herein, the term “virus-like particle” or “VLP” refers to a structure resembling a virus particle but devoid of the viral genome, incapable of replication and devoid of pathogenicity. The particle typically comprises at least one type of structural protein from a virus. Preferably only one type of structural protein is present. Most preferably no other non-structural component of a virus is present. Thus, virus-like particles can be spontaneously self-assembled by viral structural proteins under appropriate conditions in vitro while excluding the genetic material and potential replication probability. virus-like particles, with a diameter of approximately 20 to 150 nm, also have the characteristics of nanometer materials, such as large surface area, surface-accessible amino acids with reactive moieties (e.g., lysine and glutamic acid residues), inerratic spatial structure, and good biocompatibility. Therefore, assembled virus-like particles have great potential as a delivery system for specifically carrying a variety of cargos.

As used herein, the term “pseudotyped virus particle” refers to a virus particle wherein the viral envelope protein has been replaced by a heterologous protein, in particular the syncytin-1 fusion protein of the present invention.

As used herein, the term “enveloped virus particle” refers to a virus particle surrounded by a plasma membrane-derived lipid bilayer envelope. As used herein, the term “plasma membrane-derived lipid bilayer envelope” refers to a lipid bilayer derived from the plasma membrane of the host cell from which the virus particle has been released. This envelope either partially or totally encloses the virus particle. The virus particle is preferably completely (or substantially completely) enclosed within the envelope. The lipid bilayer will have a macromolecular composition corresponding to the composition of the plasma membrane of the host cell. The bilayer will have similar proportions of the same lipids, proteins and carbohydrates. Such macromolecules would include transmembrane receptors and channels (such as receptor kinases and ion channels), cytoskeletal proteins (such as actin), lipid or protein linked carbohydrates, phospholipids (such as phosphatidylcholine, phosphatidylserine and phosphatidylethanolamine), and cholesterol.

As used herein, the term “viral envelope protein” refers a protein which, in a normal enveloped virus, is encoded by the genome of the virus and is associated with the envelope of the virus, wherein the protein is e.g. capable of specifically interacting with a cognate cellular virus receptor protein to facilitate attachment of the virus to a cell. Viral envelope proteins include, but are not limited to, glycoproteins.

As used herein, the term “viral structural protein” is a protein that contributes to the overall structure of the capsid protein or the protein core of a virus. The viral structural protein of the present invention can be obtained from any virus which can form virus particles. These are typically proteins from viruses that are naturally enveloped. Such viruses include, but are not limited to, the Retroviridae (e.g. HIV, Moloney Murine Leukaemia Virus, Feline Leukaemia Virus, Rous Sarcoma Virus), the Coronaviridae, the Herpesviridae, the Hepadnaviridae, and the Orthomyxoviridae (e.g. Influenza Virus). However, naturally non-enveloped viruses may form enveloped virus particles and these are also encompassed by the invention. Naturally non-enveloped viruses include the Picomaviridae, the Reoviridae, the Adenoviridae, the Papillomaviridae and the Parvoviridae (including AAV).

As used herein, the term “Gag protein”, “GAG protein” or “group-specific antigen” refers to a family of glycoproteins that form the capsid of certain viruses. Gag proteins are processed into MA (matrix), CA (capsid), and NC (nucleocapsid) parts. Typically, the nucleocapsid protein (NC) comprises at least one zinc-finger motif flanked by highly basic regions.

As used herein, the term “target cell” means a cell with which fusion with a virus particle of the present invention is desired.

As used herein, the term “cargo” as used herein describes any molecule, e.g. nucleic acid, polypeptide, pharmaceutical, etc. with a desired biological activity and suitable solubility profile that is encapsidated into the virus particle of the present invention.

As used herein the term “encapsulation” or “encapsulated,” as used herein refers to the envelopment of a cargo within the virus particle of the present invention.

As used herein, the term “targeting moiety” refers to any molecule that binds specifically to a target.

As used herein, the term “antibody” refers to immunoglobulin molecules and immunologically active portions of immunoglobulin molecules, i.e., molecules that contain an antigen binding site that immunospecifically binds to an antigen. In natural antibodies of rodents and primates, two heavy chains are linked to each other by disulfide bonds, and each heavy chain is linked to a light chain by a disulfide bond. There are two types of light chains, lambda (l) and kappa (k). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. Accordingly, the term “variable domain” refers to the variable domain of a light chain (VL) or the variable domain of a heavy chain (VH) and thus denotes the domains which are involved directly in binding of the antibody to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate in the antibody binding site, or influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. For the antibodies described hereafter, the CDRs have been determined using CDR finding algorithms from www.bioinf.org.uk—see the section entitled How to identify the CDRs by looking at a sequence within the Antibodies pages.

As used herein, the term “immunoglobulin domain” refers to a globular region of an antibody chain (such as e.g. a chain of a conventional 4-chain antibody or of a heavy chain antibody or light chain), or to a polypeptide that essentially consists of such a globular region.

As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. “Fragments” comprise a portion of the intact antibody, generally the antigen binding site or variable region. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

As used herein, the term “single domain antibody”, “sdAb” or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

As used herein, the term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, of an agonist molecule, e.g., CD40 Ligand, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used herein, the term “humanized antibody” include antibodies which have the 6 CDRs of a murine antibody, but humanized framework and constant regions. More specifically, the term “humanized antibody”, as used herein, may include antibodies in which CDR sequences derived from the germline of another mammalian species, such as a mouse, have been grafted onto human framework sequences.

As used herein the term “human monoclonal antibody”, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, in some embodiments, the term “human monoclonal antibody”, as used herein, is not intended to include antibodies in which CDR sequences derived from the germline of another mammalian species, such as a mouse, have been grafted onto human framework sequences.

As used herein, the term “specificity” refers to the ability of an antibody to detectably bind target molecule (e.g. an epitope presented on an antigen) while having relatively little detectable reactivity with other target molecules. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

The term “affinity”, as used herein, means the strength of the binding of an antibody to a target molecule (e.g. an epitope). The affinity of a binding protein is given by the dissociation constant Kd. For an antibody said Kd is defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of a binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of binding protein is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined target molecule (e.g. an antigen or epitope) typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less.

As used herein, the term “subject”, “host”, “individual” or “patient” refers to a mammal, preferably a human being, male or female at any age that is in-need of a therapy.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

### Syncitin-1 Fusion Proteins:

The first object of the present invention relates to a fusion protein wherein a syncytin-1 polypeptide is fused to one or more targeting-moieties.

### Syncitin-1 Polypeptide:

According to the present invention, the syncytin-1 polypeptide comprises the amino acid sequence as set forth in SEQ ID NO:2 (SDGGGXXDXXR) and is capable to bind to the ASCT1 receptor, preferably to the ASCT2 receptor.

In some embodiments, the syncytin-1 polypeptide comprises the amino acid sequence as set forth in SEQ ID NO:3 (SDGGGVQDQAR).

In some embodiments, the syncytin-1 polypeptide of the present invention comprises the amino acid sequence as set forth in SEQ ID NO:3 (SDGGGVQDQAR) and comprises at least 15, 20, 25, 30, 35, 40, 45, 50, 100, 150, 200, 250, 300, 350, 400, or 450 consecutive amino acids of SEQ ID NO:1.

In some embodiments, the syncintin-1 polypeptide of the present invention comprises an amino acid sequence having at 70% of identity with the amino acid sequence that ranges from the amino acid residue at position 21 to the amino acid residue at position 480 in SEQ ID NO:1 (“SYN480”). In some embodiments, the syncintin-1 polypeptide of the present invention comprises the amino acid sequence that ranges from the amino acid residue at position 21 to the amino acid residue at position 480 in SEQ ID NO:1 wherein the arginine residue (R) at position 393 and the phenylalanine residue (F) at position 399 are mutated. In some embodiments, the syncintin-1 polypeptide of the present invention comprises the amino acid sequence that ranges from the amino acid residue at position 21 to the amino acid residue at position 480 in SEQ ID NO:1 wherein the arginine residue (R) at position 393 is substituted by a glutamine residue (Q) and the phenylalanine residue (F) are position 399 is substituted by an alanine residue (A).

In some embodiments, the syncintin-1 polypeptide of the present invention comprises an amino acid sequence having at 70% of identity with the amino acid sequence that ranges from the amino acid residue at position 21 to the amino acid residue at position 538 in SEQ ID NO:1 (“SYN”). In some embodiments, the syncintin-1 polypeptide of the present invention comprises the amino acid sequence that ranges from the amino acid residue at position 21 to the amino acid residue at position 538 in SEQ ID NO:1 wherein the arginine residue (R) at position 393 and the phenylalanine residue (F) at position 399 are mutated. In some embodiments, the syncintin-1 polypeptide of the present invention comprises the amino acid sequence that ranges from the amino acid residue at position 21 to the amino acid residue at position 538 in SEQ ID NO:1 wherein the arginine residue (R) at position 393 is substituted by a glutamine residue (Q) and the phenylalanine residue (F) are position 399 is substituted by an alanine residue (A).

### Targeting-Moieties:

According to the present invention, the targeting moiety is a polypeptide having a binding domain. The term “binding domain” as used herein refers to the one or more regions of a polypeptide that mediate specific binding with a target molecule (e.g. an antigen, ligand, receptor, substrate or inhibitor). Exemplary binding domains include an antibody variable domain, a receptor binding domain of a ligand, a ligand binding domain of a receptor or an enzymatic domain. The term “ligand binding domain” as used herein refers to any native receptor (e.g., cell surface receptor) or any region or derivative thereof retaining at least a qualitative ligand binding ability of a corresponding native receptor. The term “receptor binding domain” as used herein refers to any native ligand or any region or derivative thereof retaining at least a qualitative receptor binding ability of a corresponding native ligand. In some embodiments, the polypeptide comprises at least 1, 2, 3, 4, or 5 binding sites. The polypeptide may be either monomers or multimers. For example, in some embodiments, the polypeptide is a dimer. In some embodiments, the dimer is a homodimer, comprising two identical monomeric subunits. In some embodiments, the dimer is a heterodimer, comprising two non-identical monomeric subunits. The subunits of the dimer may comprise one or more polypeptide chains. For example, in some embodiments, the dimer comprises at least two polypeptide chains. In some embodiments, the dimer comprises two polypeptide chains. In some embodiments, the dimer comprises four polypeptide chains (e.g., as in the case of antibody molecules).

In some embodiments, the targeting moiety is a ligand. As used herein, the term “ligand” refers to a polypeptide that binds to a polypeptide receptor and typically effects a change in an activity of the receptor, and/or effects a change in conformation of the receptor, and/or affects binding of another receptor to the targeted receptor. Accordingly, a ligand comprises one or more receptor binding domain(s) as above defined. The receptor ligand is for example selected in the group consisting of a cytokine, growth factor, hormone, neuromediator, apoptosis ligand, a chemokine, glucose transporter and their combinations.

In some embodiments, the targeting moiety is an antibody or an antibody-fragment that comprises one or more variable domain(s). Typically, the antibody fragment include scFv or VHH or other functional fragment including an immunoglobulin devoid of light chains, Fab, Fab′, F(ab*) 2, Fv, antibody fragment, diabody, scAB, single-domain heavy chain antibody, single-domain light chain antibody, Fd, CDR regions, or any portion or peptide sequence of the antibody that is capable of binding antigen or epitope. Thus, in some embodiments, the polypeptide having a binding domain is a light immunoglobulin chain. In some embodiments, the polypeptide having a binding domain is a heavy immunoglobulin chain. In some embodiments, the polypeptide having a binding domain is a heavy single heavy chain variable domain of antibodies of the type that can be found in Camelid mammals which are naturally devoid of light chains. Such single domain antibody is also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388.

The techniques for preparing and using various antibody-based constructs and fragments are well known in the art (see e.g. Kohler and Milstein, Nature, 256:495, 1975).

In some embodiments, the antibody is a monoclonal antibody.

In some embodiments, the antibody is non-internalizing. As used herein the term “non-internalizing antibody” refer to an antibody, respectively, that has the property of to bind to a target antigen present on a cell surface, and that, when bound to its target antigen, does not enter the cell and become degraded in the lysosome.

In some embodiments, the targeting moiety is a non-antibody-based recognition scaffold. Non-antibody-based recognition scaffolds include, e.g., affibodies; engineered Kunitz domains; monobodies (adnectins); anticalins; designed ankyrin repeat domains (DARPins); a binding site of a cysteine-rich polypeptide (e.g., cysteine-rich knottin peptides); avimers; afflins; and the like. See, e.g., Gebauer and Skerra (2009) Curr. Opin. Chem. Biol. 13:245.

Non-antibody-based scaffolds (also referred to herein as “antibody mimic molecules”) may be identified by selection or isolation of a target-binding variant from a library of binding molecules having artificially diversified binding sites. Diversified libraries can be generated using completely random approaches (e.g., error-prone polymerase chain reaction (PCR), exon shuffling, or directed evolution) or aided by art-recognized design strategies. For example, amino acid positions that are usually involved when the binding site interacts with its cognate target molecule can be randomized by insertion of degenerate codons, trinucleotides, random peptides, or entire loops at corresponding positions within the nucleic acid which encodes the binding site (see e.g., U.S. Pub. No. 20040132028). The location of the amino acid positions can be identified by investigation of the crystal structure of the binding site in protein entity with the target molecule. Candidate positions for randomization include loops, flat surfaces, helices, and binding cavities of the binding site. Following randomization, the diversified library may then be subjected to a selection or screening procedure to obtain binding molecules with the desired binding characteristics. For example, selection can be achieved by art-recognized methods such as phage display, yeast display, or ribosome display.

In some embodiments, the non-antibody-based scaffold comprises a binding site from an affibody. Affibodies are derived from the immunoglobulin binding domains of staphylococcal Protein A (SPA) (see e.g., Nord et al., Nat. Biotechnol., 15: 772-777 (1997)). An affibody is an antibody mimic that has unique binding sites that bind specific targets. Affibodies can be small (e.g., consisting of three alpha helices with 58 amino acids and having a molar mass of about 6 kDa), have an inert format (no Fc function), and have been successfully tested in humans as targeting moieties. Affibody binding sites can be synthesized by mutagenizing an SPA-related protein (e.g., Protein Z) derived from a domain of SPA (e.g., domain B) and selecting for mutant SPA-related polypeptides having binding affinity for a target antigen or epitope. Other methods for making affibody binding sites are described in U.S. Pat. Nos. 6,740,734 and 6,602,977 and in WO 00/63243.

In some embodiments, the non-antibody-based scaffold comprises a binding site from an anticalin. An anticalin is an antibody functional mimetic derived from a human lipocalin. Lipocalins are a family of naturally-occurring binding proteins that bind and transport small hydrophobic molecules such as steroids, bilins, retinoids, and lipids. The main structure of an anticalin is similar to wild type lipocalins. The central element of this protein architecture is a beta-barrel structure of eight antiparallel strands, which supports four loops at its open end. These loops form the natural binding site of the lipocalins and can be reshaped in vitro by extensive amino acid replacement, thus creating novel binding specificities. Anticalins possess high affinity and specificity for their ligands as well as fast binding kinetics, so that their functional properties are similar to those of antibodies. Anticalins are described in, e.g., U.S. Pat. No. 7,723,476.

In some embodiments, the non-antibody-based scaffold comprises a binding site from a cysteine-rich polypeptide. Cysteine-rich domains in some embodiments do not form an alpha-helix, a beta-sheet, or a beta-barrel structure. In some embodiments, the disulfide bonds promote folding of the domain into a three-dimensional structure. In some embodiments, cysteine-rich domains have at least two disulfide bonds, e.g., at least three disulfide bonds. An exemplary cysteine-rich polypeptide is an A domain protein. A-domains (sometimes called “complement-type repeats”) contain about 30-50 or 30-65 amino acids. In some embodiments, the domains comprise about 35-45 amino acids and in some embodiments about 40 amino acids. Within the 30-50 amino acids, there are about 6 cysteine residues. Of the six cysteines, disulfide bonds typically are found between the following cysteines: C1 and C3, C2 and C5, C4 and C6. The A domain constitutes a ligand binding moiety. The cysteine residues of the domain are disulfide linked to form a compact, stable, functionally independent moiety. Clusters of these repeats make up a ligand binding domain, and differential clustering can impart specificity with respect to the ligand binding. Exemplary proteins containing A-domains include, e.g., complement components (e.g., C6, C7, C8, C9, and Factor I), serine proteases (e.g., enteropeptidase, matriptase, and corin), transmembrane proteins (e.g., ST7, LRP3, LRP5 and LRP6) and endocytic receptors (e.g. Sortilin-related receptor, LDL-receptor, VLDLR, LRP1, LRP2, and ApoER2). Methods for making A-domain proteins of a desired binding specificity are disclosed, for example, in WO 02/088171 and WO 04/044011.

In some embodiments, the non-antibody-based scaffold comprises a binding site from a repeat protein. Repeat proteins are proteins that contain consecutive copies of small (e.g., about 20 to about 40 amino acid residues) structural units or repeats that stack together to form contiguous domains. Repeat proteins can be modified to suit a particular target binding site by adjusting the number of repeats in the protein. Exemplary repeat proteins include designed ankyrin repeat proteins (i.e., a DARPins) (see e.g., Binz et al., Nat. Biotechnol., 22: 575-582 (2004)) or leucine-rich repeat proteins (i.e., LRRPs) (see e.g., Pancer et al., Nature, 430: 174-180 (2004)).

In some embodiments, the non-antibody-based scaffold comprises a DARPin. As used herein, the term “DARPin” (an acronym for designed ankyrin repeat proteins) refers to an antibody mimetic protein typically exhibiting highly specific and high-affinity target protein binding. DARPins were first derived from natural ankyrin proteins. In some embodiments, DARPins comprise three, four or five repeat motifs of an ankyrin protein. In some embodiments, a unit of an ankyrin repeat consists of 30-34 amino acid residues and functions to mediate protein-protein interactions. In some embodiments, each ankyrin repeat exhibits a helix-turn-helix conformation, and strings of such tandem repeats are packed in a nearly linear array to form helix-turn-helix bundles connected by relatively flexible loops. In some embodiments, the global structure of an ankyrin repeat protein is stabilized by intra- and inter-repeat hydrophobic and hydrogen bonding interactions. The repetitive and elongated nature of the ankyrin repeats provides the molecular bases for the unique characteristics of ankyrin repeat proteins in protein stability, folding and unfolding, and binding specificity. The molecular mass of a DARPin domain can be from about 14 or 18 kDa for four- or five-repeat DARPins, respectively. DARPins are described in, e.g., U.S. Pat. No. 7,417,130. In some embodiments, tertiary structures of ankyrin repeat units share a characteristic composed of a beta-hairpin followed by two antiparallel alpha-helices and ending with a loop connecting the repeat unit with the next one. Domains built of ankyrin repeat units can be formed by stacking the repeat units to an extended and curved structure. LRRP binding sites from part of the adaptive immune system of sea lampreys and other jawless fishes and resemble antibodies in that they are formed by recombination of a suite of leucine-rich repeat genes during lymphocyte maturation. Methods for making DARpin or LRRP binding sites are described in WO 02/20565 and WO 06/083275.

In some embodiments, the non-antibody-based scaffold comprises a binding site derived from Src homology domains (e.g. SH2 or SH3 domains), PDZ domains, beta-lactamase, high affinity protease inhibitors, or small disulfide binding protein scaffolds such as scorpion toxins. Methods for making binding sites derived from these molecules have been disclosed in the art, see e.g., Panni et al., J. Biol. Chem., 277: 21666-21674 (2002), Schneider et at, Nat. Biotechnol., 17: 170-175 (1999); Legendre et al., Protein Sci., 11:1506-1518 (2002); Stoop et al., Nat. Biotechnol., 21: 1063-1068 (2003); and Vita et al., PNAS, 92: 6404-6408 (1995). Yet other binding sites may be derived from a binding domain selected from the group consisting of an EGF-like domain, a Kringle-domain, a PAN domain, a Gla domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an Immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, a Laminin-type EGF-like domain, a C2 domain, a binding domain derived from tetranectin in its monomeric or trimeric form, and other such domains known to those of ordinary skill in the art, as well as derivatives and/or variants thereof. Exemplary non-antibody-based scaffolds, and methods of making the same, can also be found in Stemmer et al., “Protein scaffolds and uses thereof”, U.S. Patent Publication No. 20060234299 (Oct. 19, 2006) and Hey, et al., Artificial, Non-Antibody Binding Proteins for Pharmaceutical and Industrial Applications, TRENDS in Biotechnology, vol. 23, No. 10, Table 2 and pp. 514-522 (October 2005).

In some embodiments, the non-antibody-based scaffold comprises a Kunitz domain. The term “Kunitz domains” as used herein, refers to conserved protein domains that inhibit certain proteases, e.g., serine proteases. Kunitz domains are relatively small, typically being about 50 to 60 amino acids long and having a molecular weight of about 6 kDa. Kunitz domains typically carry a basic charge and are characterized by the placement of two, four, six or eight or more that form disulfide linkages that contribute to the compact and stable nature of the folded peptide. For example, many Kunitz domains have six conserved cysteine residues that form three disulfide linkages. The disulfide-rich a/s fold of a Kunitz domain can include two, three (typically), or four or more disulfide bonds. Kunitz domains have a pear-shaped structure that is stabilized the, e.g., three disulfide bonds, and that contains a reactive site region featuring the principal determinant P1 residue in a rigid confirmation. These inhibitors competitively prevent access of a target protein (e.g., a serine protease) for its physiologically relevant macromolecular substrate through insertion of the P1 residue into the active site cleft. The P1 residue in the proteinase-inhibitory loop provides the primary specificity determinant and dictates much of the inhibitory activity that particular Kunitz protein has toward a targeted proteinase. In general, the N-terminal side of the reactive site (P) is energetically more important that the P′ C-terminal side. In most cases, lysine or arginine occupy the P1 position to inhibit proteinases that cleave adjacent to those residues in the protein substrate. Other residues, particularly in the inhibitor loop region, contribute to the strength of binding. Generally, about 10-12 amino acid residues in the target protein and 20-25 residues in the proteinase are in direct contact in the formation of a stable proteinase-inhibitor protein entity and provide a buried area of about 600 to 900 A. By modifying the residues in the P site and surrounding residues Kunitz domains can be designed to target a protein of choice. Kunitz domains are described in, e.g., U.S. Pat. No. 6,057,287.

In some embodiments, the non-antibody-based scaffold is an affilin. Affilins are small antibody-mimic proteins which are designed for specific affinities towards proteins and small compounds. New affilins can be very quickly selected from two libraries, each of which is based on a different human derived scaffold protein. Affilins do not show any structural homology to immunoglobulin proteins. There are two commonly-used affilin scaffolds, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

In some embodiments, the non-antibody-based scaffold is an Avimer. Avimers are evolved from a large family of human extracellular receptor domains by in vitro exon shuffling and phage display, generating multidomain proteins with binding and inhibitory properties Linking multiple independent binding domains has been shown to create avidity and results in improved affinity and specificity compared with conventional single-epitope binding proteins. In some embodiments, Avimers consist of two or more peptide sequences of 30 to 35 amino acids each, connected by spacer region peptides. The individual sequences are derived from A domains of various membrane receptors and have a rigid structure, stabilized by disulfide bonds and calcium. Each A domain can bind to a certain epitope of the target protein. The combination of domains binding to different epitopes of the same protein increases affinity to this protein, an effect known as avidity (hence the name). Avimers with sub-nanomolar affinities have been obtained against a variety of targets. Alternatively, the domains can be directed against epitopes on different target proteins. Additional information regarding avimers can be found in U.S. patent application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756.

According to the present invention, the targeting moiety is not a protein tag. As used herein, the term “tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, to that sequence. According to the present invention, the targeting moiety does not comprise histidine residues (e.g., 4 to 8 consecutive histidine residues) that are usually added to either the amino- or carboxy-terminus of a polypeptide to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) that may be added to proteins to facilitate protein isolation by procedures such as affinity or immunoaffinity chromatography are not considered as targeting moieties according to the present invention.

According to the present invention, the targeting moiety is not a fluorescent protein. As used herein, the term “fluorescent protein” refers to the fluorescent proteins which are produced by various organisms, such as Renilla and Aequorea as well as modified forms of these native fluorescent proteins which may fluoresce in various visible colors. In general, the terms “fluorescent protein” and “GFP” are sometimes used interchangeably; however, sometimes specific other colors can be noted. The system is strictly mnemonic so that, for example, RFP refers to red fluorescent protein, YFP to yellow fluorescent protein, BFP to blue fluorescent protein, etc. A wide range of wavelength of visible light is emitted by these proteins depending on the specific modifications made.

In some embodiments, the targeting moiety is not selected from the group consisting of Biotin Carboxyl Carrier Protein (BCCP), Glutathione-S-Transferase (GST), Green Fluorescent Protein (GFP), Maltose Binding Protein (MBP), Nus-tag (NusA protein), Thioredoxin (Trx), Fc-tag (Immunoglobulin Fc domain) such as rabbit IgG, mouse IgG, goat IgG, rat IgG, bovine IgG, or dog IgG, Carbohydrate binding module (CBM), Yellow fluorescent protein, mCherry beta-galactosidase, Digoxigenin, Biotin, Small Ubiquitin-like Modifier (SUMO), AviTag, Calmodulin-tag, Polyglutamate tag, E-tag, Flag-tag, HA-tag, His-tag, Myc-tag, S-tag, SBP-tag, Strep-tag, TC-tag, V5 tag, VSV-tag, Xpress tag, Isopeptag, and Spy Tag.

In some embodiments, the targeting moiety has binding affinity to a cell surface molecule of a target cell. In some embodiments, the cell surface molecule is a receptor. In some embodiments, the cell surface molecule is a transmembrane protein. In some embodiments, the target moiety is specific for target protein antigens, carbohydrate antigens, or glycosylated proteins. For example, the antibody can target glycosylation groups of antigens that are preferentially produced by transformed (neoplastic or cancerous) cells, infected cells, and the like (cells associated with other immune system-related disorders).

A partial list of suitable mammalian cells that can be targeted by the targeting moiety of the present invention includes but are not limited to blood cells, myoblasts, bone marrow cells, peripheral blood cells, umbilical cord blood cells, cardiomyocytes (and precursors thereof), chondrocytes (cartilage cells), dendritic cells, fetal neural tissue, fibroblasts, hepatocytes (liver cells), islet cells of pancreas, keratinocytes (skin cells) and stem cells.

In some embodiments, the targeting moiety is particularly suitable for targeting a population of immune cells. Recognized populations of immune cells include lymphocytes, such as B lymphocytes (Fc receptors, MIHC class II, CD 19+, CD21+), helper T lymphocytes (CD3+, CD4+, CD8−), cytolytic T lymphocytes (CD3+, CD4−, CD8+), natural killer cells (CD16+), mononuclear phagocytes, including monocytes, neutrophils and macrophages, and dendritic cells. Other cell types that may be of interest include eosinophils and basophils.

In some embodiments, the targeting moiety is particularly suitable for targeting a population of hematopoietic cells.

As used herein, the term “hematopoietic cell” refers generally to blood cells, both from the myeloid and the lymphoid lineage. In particular, the term “hematopoietic cell” includes both undifferentiated or poorly differentiated cells such as hematopoietic stem cells and progenitor cells, and differentiated cells such as T lymphocytes, B lymphocytes or dendritic cells. Preferably, the hematopoietic cell is selected from the group consisting of hematopoietic stem cells, CD34+ progenitor cells, in particular peripheral blood CD34+ cells, very early progenitor CD34+ cells, B-cell CD19+ progenitors, myeloid progenitor CD13+ cells, T lymphocytes, B lymphocytes, monocytes, dendritic cells, cancer B cells in particular B-cell chronic lymphocytic leukemia (BOLL) cells and marginal zone lymphoma (MZL) B cells, and thymocytes.

Thus, in some embodiments, the targeting moiety is specific for an immune cell regulatory molecule such as CD3, CD4, CD8, CD25, CD28, CD26, CTLA-4, ICOS, or CD11a. Other suitable antigens include but are not limited to those associated with immune cells including T cell-associated molecules, such as TCR/CD3 or CD2; NK cell-associated targets such as NKG2D, FcγRIIIa (CD16), CD38, CD44, CD56, or CD69; granulocyte-associated targets such as FcγRI (CD64), FcαRI (CD89), and CR3 (CD11b/CD18); monocyte/macrophage-associated targets (such as FcγRI (CD64), FcαRI (CD89), CD3 (CD11b/CD18), or mannose receptor; dendritic cell-associated targets such as FcγRI (CD64) or mannose receptor; and erythrocyte-associated targets such as CRI (CD35).

In some embodiments, the targeting moiety is particularly suitable for targeting a population of malignant cells. Thus, in some embodiments, the targeting moiety is specific for a cancer antigen. Known cancer antigens include, without limitation, c-erbB-2 (erbB-2 is also known as c-neu or HER-2), which is particularly associated with breast, ovarian, and colon tumor cells, as well as neuroblastoma, lung cancer, thyroid cancer, pancreatic cancer, prostate cancer, renal cancer and cancers of the digestive tract. Another class of cancer antigens is oncofetal proteins of nonenzymatic function. These antigens are found in a variety of neoplasms, and are often referred to as “tumor-associated antigens.” Carcinoembryonic antigen (CEA), and α-fetoprotein (AFP) are two examples of such cancer antigens. AFP levels rise in patients with hepatocellular carcinoma: 69% of patients with liver cancer express high levels of AFP in their serum. CEA is a serum glycoprotein of 200 kDa found in adenocarcinoma of colon, as well as cancers of the lung and genitourinary tract. Yet another class of cancer antigens is those antigens unique to a particular tumor, referred to sometimes as “tumor specific antigens” such as heat shock proteins (e.g., hsp70 or hsp90 proteins) from a particular type of tumor. Other targets include the MICA/B ligands of NKG2D. These molecules are expressed on many types of tumors, but not normally on healthy cells. Additional specific examples of cancer antigens include epithelial cell adhesion molecule (Ep-CAM/TACSTD1), mesothelin, tumor-associated glycoprotein 72 (TAG-72), gp100, Melan-A, MART-1, KDR, RCAS1, MDA7, cancer-associated viral vaccines (e.g., human papillomavirus antigens), prostate specific antigen (PSA, PSMA), RAGE (renal antigen), CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), cancer-associated ganglioside antigens, tyrosinase, gp75, C-myc, Martl, MelanA, MUM-1, MUM-2, MUM-3, HLA-B7, Ep-CAM, tumor-derived heat shock proteins, and the like (see also, e.g., Acres et al., Curr Opin Mol Ther 2004 February, 6:40-7; Taylor-Papadimitriou et al., Biochim Biophys Acta. 1999 Oct. 8; 1455(2-3):301-13; Emens et al., Cancer Biol Ther. 2003 July-August; 2(4 Suppl 1):S161-8; and Ohshima et al., Int J Cancer. 2001 Jul. 1; 93(1):91-6). Other exemplary cancer antigen targets include CA 195 tumor-associated antigen-like antigen (see, e.g., U.S. Pat. No. 5,324,822) and female urine squamous cell carcinoma-like antigens (see, e.g., U.S. Pat. No. 5,306,811), and the breast cell cancer antigens described in U.S. Pat. No. 4,960,716.

In some embodiments, the targeting moiety has binding affinity for a pancreatic antigen. In some embodiments, the targeting moiety is specific for LP1R receptor or for IA-2 receptor that are found on type 1 diabetic pancreatic cells.

In some embodiments, the targeting moiety has binding affinity for a CD (cluster of differentiation) molecule selected from the group consisting of CD1a, CD1b, CD1c, CD1d, CD1e, CD2, CD3delta, CD3epsilon, CD3gamma, CD4, CD5, CD6, CD7, CD8alpha, CD8beta, CD9, CD10, CD11a, CD11b, CD11c, CDw12, CD13, CD14, CD15u, CD16a, CD16b, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD44R, CD45, CD46, CD47R, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD65s, CD66a, CD66b, CD66c, CD66d, CD66e, CD66f, CD68, CD69, CD70, CD71, CD72, CD73, CD74, CD75, CD75s, CD77, CD79a, CD79b, CD80, CD81, CD82, CD83, CD84, CD85, CD86, CD87, CD88, CD89, CD90, CD91, CD92, CDw93, CD94, CD95, CD96, CD97, CD98, CD99, CD100, CD101, CD102, CD103, CD104, CD105, CD106, CD107a, CD107b, CD108, CD109, CD110, CD111, CD112, CDw113, CD114, CD115, CD116, CD117, CD118, CDw119, CD120a, CD120b, CD121a, CDw121b, CD122, CD123, CD124, CDw125, CD126, CD127, CDw128a, CDw128b, CD129, CD130, CD131, CD132, CD133, CD134, CD135, CDw136, CDw137, CD138, CD139, CD140a, CD140b, CD141, CD142, CD143, CD144, CDw145, CD146, CD147, CD148, CDw149, CD150, CD151, CD152, CD153, CD154, CD155, CD156a, CD156b, CDw156C, CD157, CD158, CD159a, CD159c, CD160, CD161, CD162, CD162R, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CDw186, CD191, CD192, CD193, CD195, CD196, CD197, CDw198, CDw199, CDw197, CD200, CD201, CD202b, CD203c, CD204, CD205, CD206, CD207, CD208, CD209, CDw210, CD212, CD213a1, CD213a2, CDw217, CDw218a, CDw218b, CD220, CD221, CD222, CD223, CD224, CD225, CD226, CD227, CD228, CD229, CD230, CD231, CD232, CD233, CD234, CD235a, CD235b, CD235ab, CD236, CD236R, CD238, CD239, CD240CE, CD240D, CD240DCE, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD271, CD272, CD273, CD274, CD275, CD276, CD277, CD278, CD279, CD280, CD281, CD282, CD283, CD284, CD289, CD292, CDw293, CD294, CD295, CD296, CD297, CD298, CD299, CD300a, CD300c, CD300e, CD301, CD302, CD303, CD304, CD305, CD306, CD307, CD309, CD312, CD314, CD315, CD316, CD317, CD318, CD319, CD320, CD321, CD322, CD324, CDw325, CD326, CDw327, CDw328, CDw329, CD331, CD332, CD333, CD334, CD335, CD336, CD337, CDw338, and CD339.

In some embodiments, the targeting moiety has biding affinity for a cell surface molecule of the hematopoietic lineage. In some embodiments, the targeting moiety has biding affinity for a cell surface molecule selected from the group consisting of 2B4/CD244/SLAMF4, ABCG2, Aldehyde Dehydrogenase 1-A1/ALDH1A1, BMI-1, C1qR1/CD93, CD34, CD38, CD44, CD45, CD48/SLAMF2, CD90/Thy1, CD117/c-kit, CD133, CDCP1, CXCR4, Endoglin/CD105, EPCR, Erythropoietin R, ESAM, EVI-1, Integrin alpha 6/CD49f, SLAM/CD150, VCAM-1/CD106 and VEGFR2/KDR/Flk-1.

In some embodiments, the targeting moiety is the Stem Cell Factor (SCF, also known as kit ligand (KITL)), which binds CD117 (c-kit) receptor. In some embodiments, the targeting moiety thus comprises an amino acid sequence having at least 70% of identity with the amino acid sequence as set forth in SEQ ID NO:4.

SEQ ID NO: 4 >sp|P21583|SCF_HUMAN Kit ligand OS = Homo sapiens OX = 9606
GN = KITLG P E= 1 SV = 1 (soluble form)
CRNRVTNNVKDVTKLVANLPKDYMITLKYVPGMDVLPSHCWISEMVVQLSDSLTDLLDKESNISEGLSN
YSIIDKLVNIVDDLVECVKENSSKDLKKSFKSPEPRLFTPEEFFRIENRSIDAFKDFVVASETSDCVVS
STLSPEKDSRVSVTKPFMLPPVAASSLRNDSSSSNRKAKNPPGDSSLHWAAMALPALFSLIIGFAFGAL
YWKKR

In some embodiments, the targeting moiety is a single-chain fragment variant (scFv) directed against CD133 receptor (“scFvCD133”). In some embodiments, the targeting moiety thus comprises an amino acid sequence having at least 70% of identity with the amino acid sequence as set forth in SEQ ID NO:5.

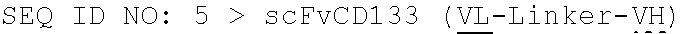

MDIVLSQSPAIMSASPGEKVTISCSASSSVSYMYWYQQKPGSSPKPWIYRTSNLASGVPARFSGSGSGT

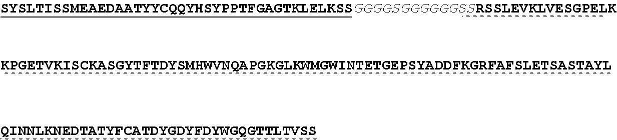

In some embodiments, the targeting moiety is a DARPin directed against CD4 (“DARPinCD4”). In some embodiments, the targeting moiety thus comprises an amino acid sequence having at least 70% of identity with the amino acid sequence as set forth in SEQ ID NO:6.

>DARPinCD4
SEQ ID NO : 6
AAQPADLGKKLLEAARAGQDDEVRILMANGADVNATDTLGRTPLHLAAQN
GHLEIVEVLLKHSADVNAIEEVGMTPLHLAVVAGHLEIVEVLLKNGADVN
AQDKFGKTAFDISIDYGNEDLAEILQKLN

In some embodiments, the targeting moiety is a single-chain fragment variant (scFv) directed against CD8 (“scFvCD8”). In some embodiments, the targeting moiety thus comprises an amino acid sequence having at least 70% of identity with the amino acid sequence as set forth in SEQ ID NO:7.

>scFvCD8
SEQ ID NO : 7
AAQPAQVQLVQSGAEDKKPGASVKVSCKASGENIKDTYIHWVRQAPGQGL
EWMGRIDPANDNTLYASKFQGRVTITADTSSNTAYMELSSLRSEDTAVYY
CGRGYGYYVFDHWGQGTTVTVSSGGGGSGGGGSGGGGSDIVMTQSPSSLS
ASVGDRVTITCRTSRSISQYLAWYQEKPGKAPKLLIYSGSTLQSGVPSRF
SGSGSGTDFTLTISSLQPEDFATYYCQQHNENPLTFGQGTKVEIKR

In some embodiments, the targeting moiety is a single-chain fragment variant (scFv) directed against IA-2 receptor (“scFvIA-2”). In some embodiments, the targeting moiety thus comprises an amino acid sequence having at least 70% of identity with the amino acid sequence as set forth in SEQ ID NO:8.

>scFVIA-2
SEQ ID NO : 8
QVQLQESGPGLVKPSETLSLRCNVSGVSISGFYWGWIRQPPGKGLEWIGH
IFSSGSTDYSPSLKSRVDVSMDTSKNHFSLNLSSVTAADTAVYYCARGLK
GVATASFDFWGRGTLVTVSSSSGGGGSGGGGSGGGGSYVLTQPPSVSVAP
GKTATITCGADNIGTKSVHWYQQRPGQAPMLVIYYNKNRPSGIPERFSGS
NSGHTATLTISRVEAGDEAAYYCQVWDTRSDLVVFGGGTKLTVLG

In some embodiments, the targeting moiety is GLP1 (“GLP1”). In some embodiments, the targeting moiety thus comprises an amino acid sequence having at least 70% of identity with the amino acid sequence as set forth in SEQ ID NO:9.

>GLP1
SEQ ID NO : 9
HAEGTFTSDVSSYLEGQAAKEFIAWLVKGRG

### Fusion:

In some embodiments, the C-terminal end of the targeting moiety is fused to the N-terminal end of the sycintin-1 polypeptide.

In some embodiments, the syncytin-1 polypeptide of the present invention and the targeting moiety are fused to each other directly (i.e. without use of a linker) or via a linker. The linker is typically a linker peptide and will, according to the invention, be selected so as to allow binding of the polypeptide to the targeting moiety. Suitable linkers will be clear to the skilled person based on the disclosure herein, optionally after some limited degree of routine experimentation. Suitable linkers are described herein and may—for example and without limitation—comprise an amino acid sequence, which amino acid sequence preferably has a length of 2 or more amino acids. Typically, the linker has 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. However, the upper limit is not critical but is chosen for reasons of convenience regarding e.g. biopharmaceutical production of such fusion proteins. The linker sequence may be a naturally occurring sequence or a non-naturally occurring sequence. If used for therapeutical purposes, the linker is preferably non-immunogenic in the subject to which the fusion protein of the present invention is administered. One useful group of linker sequences are linkers derived from the hinge region of heavy chain antibodies as described in WO 96/34103 and WO 94/04678. Other examples are poly-alanine linker sequences such as Ala-Ala-Ala. Further preferred examples of linker sequences are Gly/Ser linkers of different length including (gly4ser)3, (gly4ser)4, (gly4ser), (gly3ser), gly3, and (gly3ser2)3.

In some embodiments, the syncytin-1 polypeptide is fused to 2, 3, 4, 5, 6, 7 or 8 targeting-moieties that can be fused to each other either directly or indirectly by a linker.

In some embodiments, the fusion protein comprises the sequence of a signal peptide. As used herein, the term “signal peptide” has its general meaning in the art and refers to a pre-peptide which is present as an N-terminal peptide on a precursor form of a protein. The function of the signal peptide is to facilitate translocation of the expressed polypeptide to which it is attached into the endoplasmic reticulum. The signal peptide is normally cleaved off in the course of this process. The signal peptide may be heterologous or homologous to the organism used to produce the polypeptide.

In some embodiments, the signal peptide is the Syncytin-1 (SYN) signal sequence (SS). In some embodiments, the signal peptide consists of the amino acid sequence that ranges from the amino acid residue at position to the amino acid residue at position 20 in SEQ ID NO:1.

In some embodiments, the C-terminal end of the signal peptide is fused to the N-terminal end of the targeting moiety.

In some embodiments, the syncytin-1 fusion protein of the present invention thus comprises in the following order, the Syncytin-1 (SYN) signal sequence (SS), the targeting moiety and the syncytin-1 polypeptide.

In some embodiments, the syncytin-1 fusion protein of the present invention thus comprises a Tag. In some embodiments, the Tag is the HA epitope and consists of the amino acid sequence as set forth in SEQ ID NO: 40.

SEQ ID NO: 41 > HA epitope

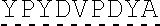

### Specific Embodiments of the Synctin-1 Fusion Protein:

In some embodiments, the synctin-1 fusion protein of the present invention consists of the amino acid sequence as set forth in SEQ ID NO:10 (“SCF-SYN”), SEQ ID NO:11 (“scFvCD133-SYN”), SEQ ID NO:12 (“DARPinCD4-SYN”), SEQ ID NO:13 (“scFVCD8-SYN”) or SEQ ID NO:14 (“scFVIA-2-SYN”), SEQ ID NO:15 (“GLP1-SYN”).

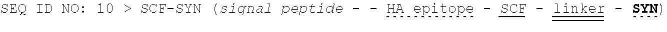

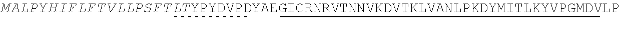

SHCWISEMVVQLSDSLTDLLDKFSNISEGLSNYSIIDKLVNIVDDLVECVKENSSKDLKKSFKSPEPRL
FTPEEFFRIFNRSIDAFKDFVVASETSDCVVSSTLSPEKDSRVSVTKPFMLPPVAAGGGSGGGGSGGGS

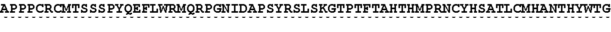

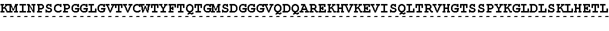

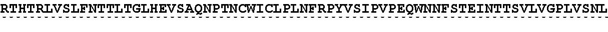

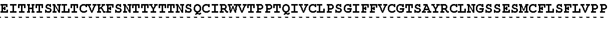

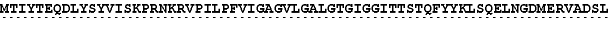

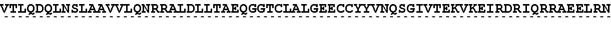

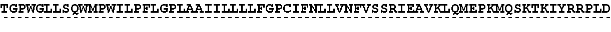

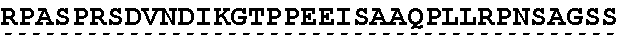

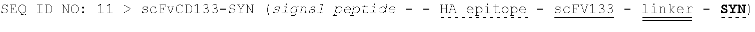

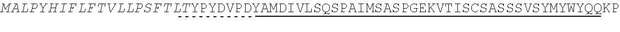

GSSPKPWIYRTSNLASGVPARFSGSGSGTSYSLTISSMEAEDAATYYCQQYHSYPPTFGAGTKLELKSS
GGGGSGGGGGGSSRSSLEVKLVESGPELKKPGETVKISCKASGYTFTDYSMHWVNQAPGKGLKWMGWIN
TETGEPSYADDFKGRFAFSLETSASTAYLQINNLKNEDTATYFCATDYGDYFDYWGQGTTLTVSSGGGS

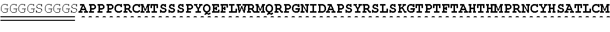

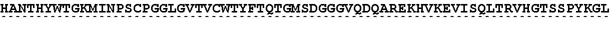

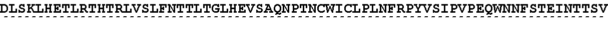

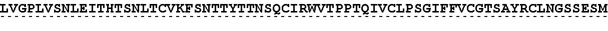

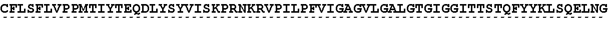

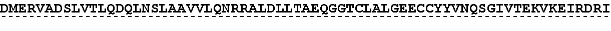

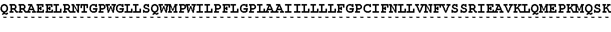

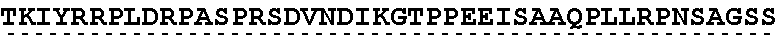

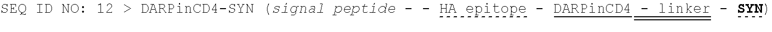

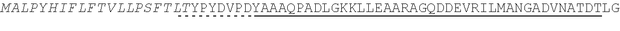

RTPLHLAAQNGHLEIVEVLLKHSADVNAIEEVGMTPLHLAVVAGHLEIVEVLLKNGADVNAQDKFGKTA

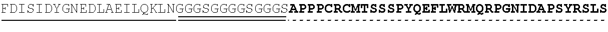

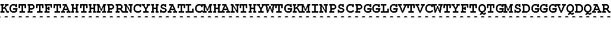

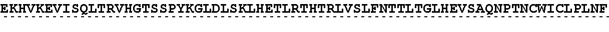

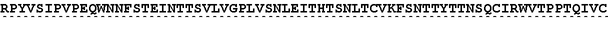

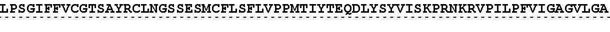

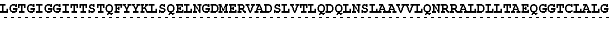

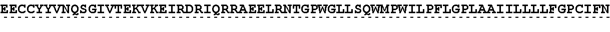

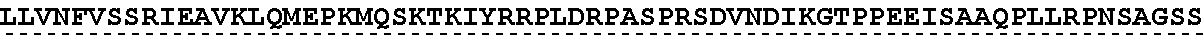

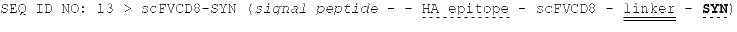

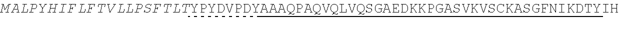

WVRQAPGQGLEWMGRIDPANDNTLYASKFQGRVTITADTSSNTAYMELSSLRSEDTAVYYCGRGYGYYV
FDHWGQGTTVTVSSGGGGSGGGGSGGGGSDIVMTQSPSSLSASVGDRVTITCRTSRSISQYLAWYQEKP
GKAPKLLIYSGSTLQSGVPSRFSGSGSGTDFTLTISSLQPEDFATYYCQQHNENPLTFGQGTKVEIKRG

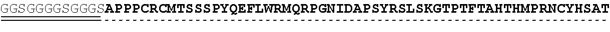

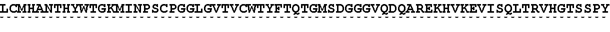

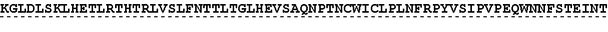

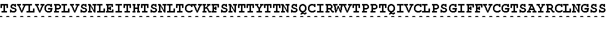

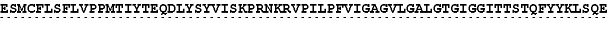

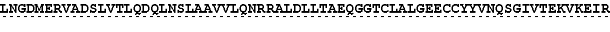

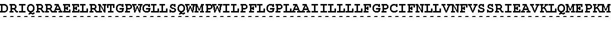

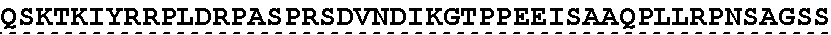

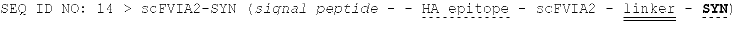

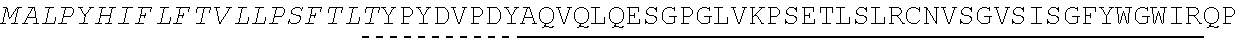

PGKGLEWIGHIFSSGSTDYSPSLKSRVDVSMDTSKNHFSLNLSSVTAADTAVYYCARGLKGVATASFDF
WGRGTLVTVSSSSGGGGSGGGGSGGGGSYVLTQPPSVSVAPGKTATITCGADNIGTKSVHWYQQRPGQA
PMLVIYYNKNRPSGIPERFSGSNSGHTATLTISRVEAGDEAAYYCQVWDTRSDLVVFGGGTKLTVLGGG
GSGGGGSGGGSAPPPCRCMTSSSPYQEFLWRMQRPGNIDAPSYRSLSKGTPTFTAHTHMPRNCYHSATL
CMHANTHYWTGKMINPSCPGGLGVTVCWTYFTQTGMSDGGGVQDQAREKHVKEVISQLTRVHGTSSPYK
GLDLSKLHETLRTHTRLVSLFNTTLTGLHEVSAQNPTNCWICLPLNFRPYVSIPVPEQWNNFSTEINTT
SVLVGPLVSNLEITHTSNLTCVKFSNTTYTTNSQCIRWVTPPTQIVCLPSGIFFVCGTSAYRCLNGSSE
SMCFLSFLVPPMTIYTEQDLYSYVISKPRNKRVPILPFVIGAGVLGALGTGIGGITTSTQFYYKLSQEL
NGDMERVADSLVTLQDQLNSLAAVVLQNRRALDLLTAEQGGTCLALGEECCYYVNQSGIVTEKVKEIRD
RIQRRAEELRNTGPWGLLSQWMPWILPFLGPLAAIILLLLFGPCIFNLLVNFVSSRIEAVKLQMEPKMQ
SKTKIYRRPLDRPASPRSDVNDIKGTPPEEISAAQPLLRPNSAGSS

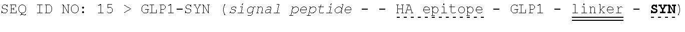

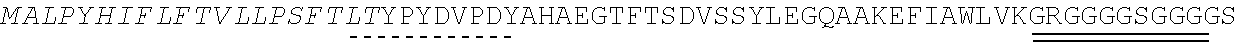

GGGSAPPPCRCMTSSSPYQEFLWRMQRPGNIDAPSYRSLSKGTPTFTAHTHMPRNCYHSATLCMHANTH
YWTGKMINPSCPGGLGVTVCWTYFTQTGMSDGGGVQDQAREKHVKEVISQLTRVHGTSSPYKGLDLSKL
HETLRTHTRLVSLFNTTLTGLHEVSAQNPTNCWICLPLNFRPYVSIPVPEQWNNESTEINTTSVLVGPL
VSNLEITHTSNLTCVKFSNTTYTTNSQCIRWVTPPTQIVCLPSGIFFVCGTSAYRCLNGSSESMCFLSF
LVPPMTIYTEQDLYSYVISKPRNKRVPILPFVIGAGVLGALGTGIGGITTSTQFYYKLSQELNGDMERV
ADSLVTLQDQLNSLAAVVLQNRRALDLLTAEQGGTCLALGEECCYYVNQSGIVTEKVKEIRDRIQRRAE
ELRNTGPWGLLSQWMPWILPFLGPLAAIILLLLFGPCIFNLLVNFVSSRIEAVKLQMEPKMQSKTKIYR
RPLDRPASPRSDVNDIKGTPPEEISAAQPLLRPNSAGSS

In some embodiments, the synctin-1 fusion protein of the present invention consists of the amino acid sequence as set forth in SEQ ID NO:16 (“SCF-SYN480”), SEQ ID NO:117 (“scFvCD133-SYN480”), SEQ ID NO:18 (“DARPinCD4-SYN480”), SEQ ID NO: 19 (“scFVCD8-SYN480”) or SEQ ID NO:20 (“scFVIA-2-SYN480”) or SEQ ID NO:21 (“GLPT-SYN480”).

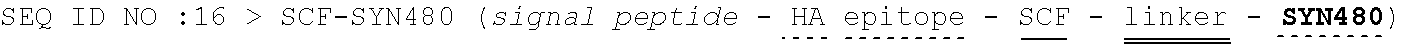

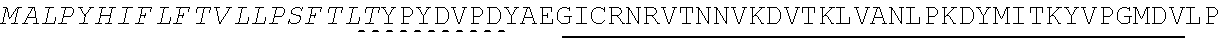

SHCWISEMVVQLSDSLTDLLDKESNISEGLSNYSIIDKLVNIVDDLVECVKENSSKDLKKSFKSPEPRL
FTPEEFFRIFNRSIDAFKDFVVASETSDCVVSSTLSPEKDSRVSVTKPFMLPPVAAGGGSGGGGSGGGS
APPPCRCMTSSSPYQEFLWRMQRPGNIDAPSYRSLSKGTPTFTAHTHMPRNCYHSATLCMHANTHYWTG

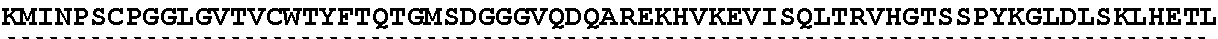

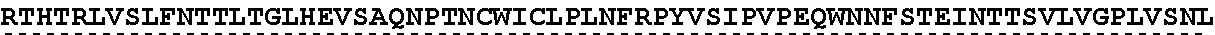

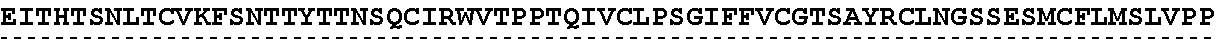

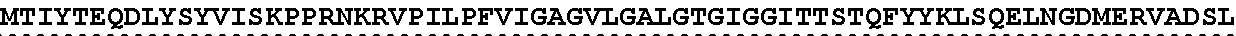

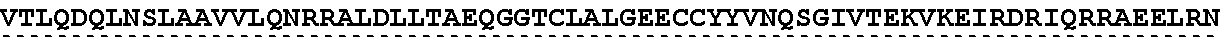

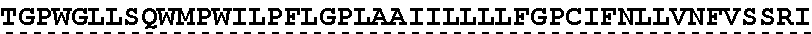

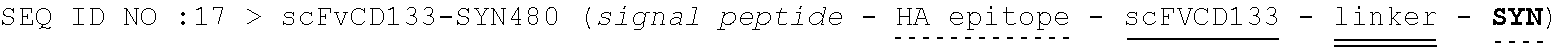

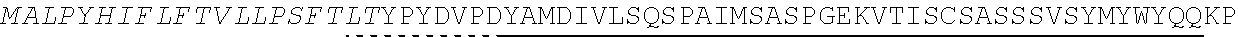

GSSPKPWIYRTSNLASGVPARFSGSGSGTSYSLTISSMEAEDAATYYCQQYHSYPPTFGAGTKLELKSS
GGGGSGGGGGGSSRSSLEVKLVESGPELKKPGETVKISCKASGYTFTDYSMHWVNQAPGKGLKWMGWIN
TETGEPSYADDFKGRFAFSLETSASTAYLQINNLKNEDTATYFCATDYGDYFDYWGQGTTLTVSSGGGS

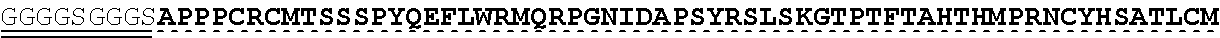

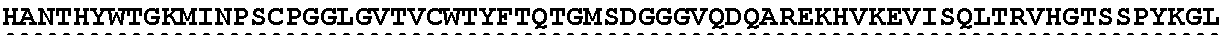

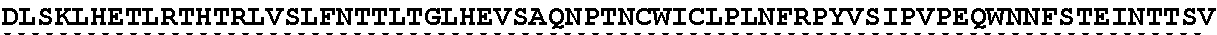

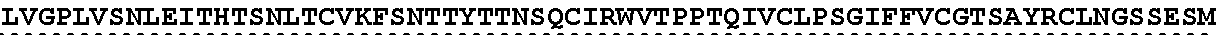

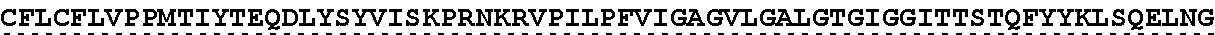

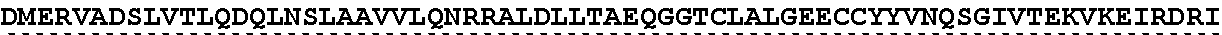

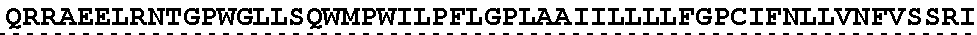

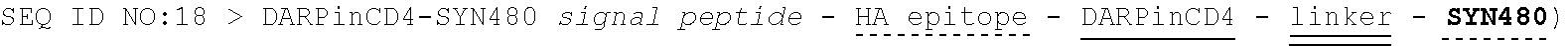

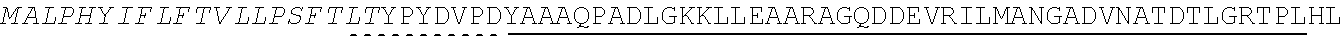

AAQNGHLEIVEVLLKHSADVNAIEEVGMTPLHLAVVAGHLEIVEVLLKNGADVNAQDKFGKTAFDISIDYGNEDL

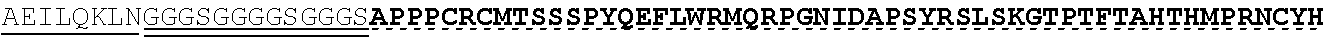

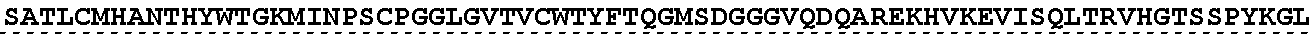

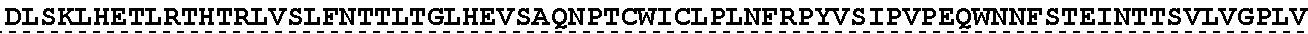

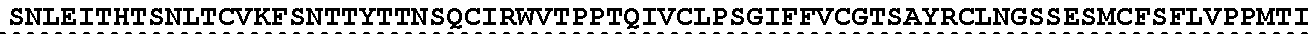

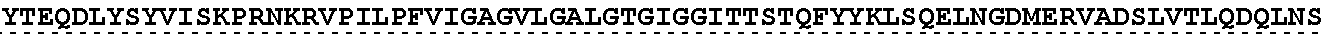

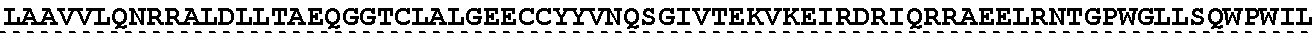

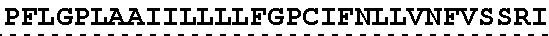

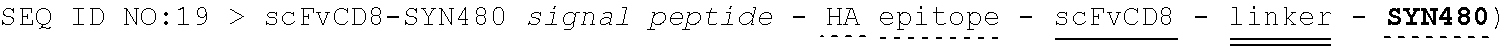

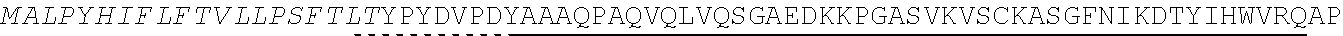

GQGLEWMGRIDPANDNTLYASKFQGRVTITADTSSNTAYMELSSLRSEDTAVYYCGRGYGYYVFDHWGQGTTVTV
SSGGGGSGGGGSGGGGSDIVMTQSPSSLSASVGDRVTITCRTSRSISQYLAWYQEKPGKAPKLLIYSGSTLQSGV
PSRFSGSGSGTDFTLTISSLQPEDFATYYCQQHNENPLTFGQGTKVEIKRGGGSGGGGSGGGSAPPPCRCMTSSS
PYQEFLWRMQRPGNIDAPSYRSLSKGTPTFTAHTHMPRNCYHSATLCMHANTHYWTGKMINPSCPGGLGVTVCWT
YFTQTGMSDGGGVQDQAREKHVKEVISQLTRVHGTSSPYKGLDLSKLHETLRTHTRIVSLENTTLTGLHEVSAQN
PTNCWICLPLNFRPYVSIPVPEQWNNFSTEINTTSVLVGPLVSNLEITHTSNLTCVKESNTTYTTNSQCIRWVTP
PTQIVCLPSGIFFVCGTSAYRCLNGSSESMCFLSFLVPPMTIYTEQDLYSYVISKPRNKRVPILPFVIGAGVLGA
LGTGIGGITTSTQFYYKLSQELNGDMERVADSLVTLQDQLNSLAAVVLQNRRALDLLTAEQGGTCLALGEECCYY
VNQSGIVTEKVKEIRDRIQRRAEELRNTGPWGLLSQWMPWILPFLGPLAAIILLLLEGPCIFNLLVNFVSSRI

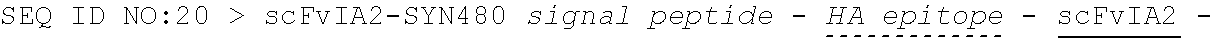

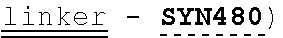

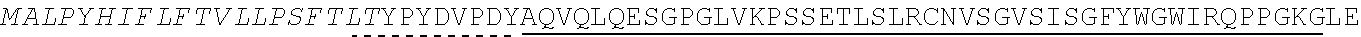

WIGHIFSSGSTDYSPSLKSRVDVSMDTSKNHFSLNLSSVTAADTAVYYCARGLKGVATASFDFWGRGTLVTVSSS
SGGGGSGGGGSGGGGSYVLTQPPSVSVAPGKTATITCGADNIGTKSVHWYQQRPGQAPMLVIYYNKNRPSGIPER
FSGSNSGHTATLTISRVEAGDEAAYYCQVWDTRSDLVVFGGGTKLTVLGGGGSGGGGSGGGSAPPPCRCMTSSSP
YQEFLWRMQRPGNIDAPSYRSLSKGTPTFTAHTHMPRNCYHSATLCMHANTHYWTGKMINPSCPGGLGVTVCWTY
FTQTGMSDGGGVQDQAREKHVKEVISQLTRVHGTSSPYKGLDLSKLHETLRTHTRLVSLENTTLTGLHEVSAQNP
TNCWICLPLNFRPYVSIPVPEQWNNFSTEINTTSVLVGPLVSNLEITHTSNLTCVKFSNTTYTTNSQCIRWVTPP
TQIVCLPSGIFFVCGTSAYRCLNGSSESMCFLSFLVPPMTIYTEQDLYSYVISKPRNKRVPILPFVIGAGVLGAL
GTGIGGITTSTQFYYKLSQELNGDMERVADSLVTLQDQLNSLAAVVLQNRRALDLLTAEQGGTCLALGEECCYYV
NQSGIVTEKVKEIRDRIQRRAEELRNTGPWGLLSQWMPWILPFLGPLAAIILLLLFGPCIENLLVNFVSSRI

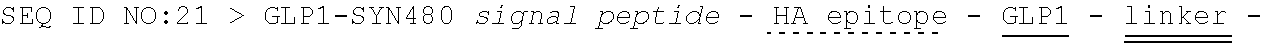

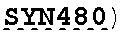

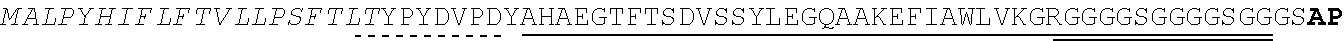

PPCRCMTSSSPYQEFLWRMQRPGNIDAPSYRSLSKGTPTFTAHTHMPRNCYHSATLCMHANTHYWTGKMINPSCP
GGLGVTVCWTYFTQTGMSDGGGVQDQAREKHVKEVISQLTRVHGTSSPYKGLDLSKLHETLRTHTRLVSLENTTL
TGLHEVSAQNPTNCWICLPLNFRPYVSIPVPEQWNNFSTEINTTSVLVGPLVSNLEITHTSNLTCVKFSNTTYTT
NSQCIRWVTPPTQIVCLPSGIFFVCGTSAYRCLNGSSESMCFLSFLVPPMTIYTEQDLYSYVISKPRNKRVPILP
FVIGAGVLGALGTGIGGITTSTQFYYKLSQELNGDMERVADSLVTLQDQLNSLAAVVLQNRRALDLLTAEQGGTC
LALGEECCYYVNQSGIVTEKVKEIRDRIQRRAEELRNTGPWGLLSQWMPWILPFLGPLAAIILLLLFGPCIENLL
VNFVSSRI

### Nucleic Acids, Vectors and Host Cells of the Present Invention:

A further object of the invention relates to a polynucleotide that encodes for the syncytin-1 fusion protein of the present invention.

Typically, said polynucleotide is a DNA or RNA molecule, which may be included in any suitable vector, such as a plasmid, cosmid, episome, artificial chromosome, phage or a viral vector.

So, a further object of the invention relates to a vector comprising a polynucleotide of the present invention.

As used herein, the terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence.

Such vectors may comprise regulatory sequences, such as a promoter, enhancer, terminator and the like, to cause or direct expression of said antibody upon administration to a subject.

As used herein, the term “regulatory sequence” refers to a nucleic acid sequence (such as, for example, a DNA sequence) recognized by the synthetic machinery of the cell, or introduced synthetic machinery, required to initiate the specific transcription of a polynucleotide sequence, thereby allowing the expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the human antibody C region can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a host cell which has been transfected, infected or transformed by the polynucleotide and/or a vector according to the invention.

As used herein, the term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

The polynucleotides of the invention may be used to produce the syncitin-1 fusion protein of the present invention in a suitable expression system. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts. Mammalian host cells include Chinese Hamster Ovary (CHO cells) including dhfr− CHO cells (described in 1) used with a DHFR selectable marker, CHOK1 dhfr+ cell lines, NSO myeloma cells, COS cells and SP2 cells, for example GS CHO cell lines together with GS Xceed™ gene expression system (Lonza), or HEK cells.

The present invention also relates to a method of producing a recombinant host cell expressing the syncytin-1 fusion protein of the present invention, said method comprising the steps of (i) introducing in vitro or ex vivo a recombinant polynucleotide or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

The host cell as disclosed herein are thus particularly suitable for producing the syncitin-1 fusion protein of the present invention. Indeed, when recombinant expression are introduced into mammalian host cells, the polypeptides are produced by culturing the host cells for a period of time sufficient for expression of the antibody in the host cells and, optionally, secretion of the antibody into the culture medium in which the host cells are grown. The antibodies can be recovered and purified for example from the culture medium after their secretion using standard protein purification methods.

Particles Functionalized with Syncitin-1 Fusion Proteins:

The present invention relates to a particle functionalized with the syncytin-1 fusion protein of the present invention.

The syncytin-1 fusion protein of the present invention is particularly suitable for allowing the i) the fusion of the particle to the membrane of the target cell by the syncytin-1 polypeptide and ii) the specific targeting to the target cell by the targeting moiety(ies).

Any particles which have been described in the art for cargo delivery into cells may be used. Such nanoparticles include for example liposomes and micelles, nanosphere or nanoparticles, nanotubes, nanocrystals, hydrogels, carbon-based nanoparticles and the like. In addition to the above, biological particles can also be utilised as particles in accordance with the present invention. Examples of these include viral particles (which normally have a size of 20 nm to 300 nm), vims-like particles (e.g. particles that are composed of only the shell of a viral particle), HDL and LDL nanoparticles (which normally have a size of 5-30 nm), self-assembled nanoparticles, 1 bacterial particles, and cells. Any such particles that can be functionalized with the syncintin-1 fusion protein can be used in accordance with the present invention. The particles can act as a carrier to carry one or more cargo(s), and bind to a target cell to release the cargo(s) inside the cell.

In some embodiments, the particle is a nanoparticle that a mean diameter between 1 to 2000 nm diameter, for example between 10 to 500 nm or between 10 to 200 nm. For most nanoparticles, the size of the nanoparticles is the distance between the two most distant points in the nanoparticle. Nanoparticle size can be determined by different methods such as Dynamic Light Scattering (DLS), Small Angle X-ray Scattering (SAXS), Scanning Mobility Particle Sizer (SMPS), Scanning Electron Microscopy (SEM), Transmission Electron Microscopy (TEM) (Orts-Gil, G., K. Natte, et al. (2011), Journal of Nanoparticle Research 13(4): 1593-1604; Alexandridis, P. and B. Lindman (2000), Amphiphilic Block Copolymers: Self-Assembly and Applications, Elsevier Science; Hunter, R. J. and L. R. White (1987). Foundations of colloid science, Clarendon Press.).

In other embodiments, the nanoparticles comprises at least a core with one or more polymers, or their copolymer, such as, e.g., one or more of dextran, carboxymethyl dextran, chitosan, trimetylchitosan, polyvinylalcohol (PVA), polyanhydrides, polyacylates, polymethacrylates, polyacylamides, cellulose, hydromellose, starch, dendrimers, polyamino acids, polyethyleneglycols, polyethyleneglycol-co-propyleneglycol, aliphatic polyesters, including poly(lactic acid (PLA), poly(glycolic acid), and their copolymers including poly(lactic-co-glycolylic)acid (PLGA), or poly(ε-caprolactone). Other suitable polymers may comprise polyamino acid selected from the group consisting of poly(g-glutamic acid), poly(a-aspartic acid), poly(e-lysine), poly(a-glutamic acid), poly(a-lysine), poly-asparagine, or derivatives thereof, and mixtures thereof. In general the surface of the nanoparticles may also be functionalised or coated to produce a desirable physical characteristic such as solubility, biocompatibility, and for facilitating chemical linkages with other biomolecules, such as the syncytin-1 fusion protein of the present invention. In some embodiments, the surface of the nanoparticles can be functionalized by incorporating one or more chemical linkers such as, without limitation: carboxyl groups, amine groups, carboxyl/amine, hydroxyl groups, polymers such as silane, dextran or PEG or their derivatives.

In some embodiments, the particle is a virus particle, more particularly a virus-like particle pseudotyped with the syncitin-1 fusion protein of the present invention.

In some embodiments, the virus particle of the present invention is an enveloped virus particle.

In some embodiments, the virus particle of the present invention comprises one or more viral structural proteins.

Preferred structural proteins are the Retroviridae Gag proteins. Particularly preferred as the structural protein is the protein corresponding to the HIV-1 gag gene. This is because the production and assembly of Gag virus particles is highly efficient and these virus particles have low cytotoxicity. The gag gene of the lentivirus HIV-1 codes for the polyprotein Pr55Gag which is a precursor of the structural proteins p17 matrix (MA), p24 capsid (CA), p7 nucleocapsid (NC) and p6. Gag is cleaved into the individual proteins in mature, infectious virions of HIV-1, however, in Gag virus particles Gag remains as a single protein since the required viral protease is absent. The mechanisms underlying and proteins involved in Gag virus particle formation are extensively discussed in the prior art (see Carriere et al., 1995 J. Virol. 69:2366-2377; Wilk et al., 2001 J. Virol. 75:759-77130; US2002/0052040; Chazal and Gerlier, 2003 Microbiol. Molec. Biol. Rev. 67:226-237; Hong and Boulanger, 1993 J, Virol. 67:2787-2798; Royer et al., 1992 J. Virol. 66:3230-3235; Spearman et al, 1994 J. Virol. 68:3232-3242 and references cited therein).

Thus, in some the virus particle of the present invention comprises a Gag protein, and most preferably a Gag protein originating from a virus selected in a group comprising Rous Sarcoma Virus (RSV) Feline Immunodeficiency Virus (FIV), Simian Immunodeficiency Virus (SIV), Moloney Leukemia Virus (MLV) and Human Immunodeficiency Viruses (HIV-1 and HIV-2) especially Human Immunodeficiency Virus of type 1 (HIV-1).

As it is readily understood by the one skilled in the art, a virus particle that is used according to the invention may be selected in a group comprising Moloney murine leukemia virus-derived vector particles, Bovine immunodeficiency virus-derived particles, Simian immunodeficiency virus-derived vector particles, Feline immunodeficiency virus-derived vector particles, Human immunodeficiency virus-derived vector particles, Equine infection anemia virus-derived vector particles, Caprine arthritis encephalitis virus-derived vector particle, Baboon endogenous virus-derived vector particles, Rabies virus-derived vector particles, Influenza virus-derived vector particles, Norovirus-derived vector particles, Respiratory syncytial virus-derived vector particles, Hepatitis A virus-derived vector particles, Hepatitis B virus-derived vector particles, Hepatitis E virus-derived vector particles, Newcastle disease virus-derived vector particles, Norwalk virus-derived vector particles, Parvovirus-derived vector particles, Papillomavirus-derived vector particles, Yeast retrotransposon-derived vector particles, Measles virus-derived vector particles, and bacteriophage-derived vector particles.

In some embodiments, the virus particle of the present invention is a retrovirus-derived particle. In some embodiments, the virus particle of the present invention is a lentivirus-derived particle. Lentiviruses belong to the retrovirus's family, and have the unique ability of being able to infect non-dividing cells. Such lentivirus may be selected among Bovine immunodeficiency virus, Simian immunodeficiency virus, Feline immunodeficiency virus, Human immunodeficiency virus, Equine infection anemia virus, and Caprine arthritis encephalitis virus. For preparing Moloney murine leukemia virus-derived vector particles, the one skilled in the art may notably refer to the methods disclosed by Sharma et al. (1997, Proc Natl Acad Sci USA, Vol. 94: 10803+−10808), Guibingua et al. (2002, Molecular Therapy, Vol. 5 (no 5): 538-546). Moloney murine leukemia virus-derived (MLV-derived) vector particles may be selected in a group comprising MLV-A-derived vector particles and MLV-E-derived vector particles. For preparing Bovine Immunodeficiency virus-derived vector particles, the one skilled in the art may notably refer to the methods disclosed by Rasmussen et al. (1990, Virology, Vol. 178 (no 2): 435-451). For preparing Simian immunodeficiency virus-derived vector particles, the one skilled in the art may notably refer to the methods disclosed by Mangeot et al. (2000, Journal of Virology, Vol. 71 (no 18): 8307-8315), Negre et al. (2000, Gene Therapy, Vol. 7: 1613-1623) Mangeot et al. (2004, Nucleic Acids Research, Vol. 32 (no 12), e102). For preparing Feline Immunodeficiency virus-derived vector particles, the one skilled in the art may notably refer to the methods disclosed by Saenz et al. (2012, Cold Spring Harb Protoc, (1): 71-76; 2012, Cold Spring Harb Protoc, (1): 124-125; 2012, Cold Spring Harb Protoc, (1): 118-123). For preparing Human immunodeficiency virus-derived vector particles, the one skilled in the art may notably refer to the methods disclosed by Jalaguier et al. (2011, PlosOne, Vol. 6 (no 11), e28314), Cervera et al. (J Biotechnol, Vol. 166 (no 4): 152-165), Tang et al. (2012, Journal of Virology, Vol. 86 (no 14): 7662-7676). For preparing Equine infection anemia virus-derived vector particles, the one skilled in the art may notably refer to the methods disclosed by Olsen (1998, Gene Ther, Vol. 5 (no 11): 1481-1487). For preparing Caprine arthritis encephalitis virus-derived vector particles, the one skilled in the art may notably refer to the methods disclosed by Mselli-Lakhal et al. (2006, J Virol Methods, Vol. 136 (no 1-2): 177-184).

In some embodiments, capsids from mammalian endogenous retrovirus are used. Among these are homologs of the capsid protein (known as Gag) of long terminal repeat (LTR) retrotransposons and retroviruses. Recently, several mammalian Gag homologs that form virus particles were identified (Campillo et al. 2006 PMID: 16979784 (computation analysis); Pastuzyn et al. 2018 PMID: 29328916 (ARC); Ashley et al. 2018 PMID: 29328915 (ARC) and Abed et al. 2019 PMID: 30951545 (10)). For instance, Arc, MOAP1, ZCCHC12, RTL1, PNMA3, PNMA5, PNMA6a, and PEG10 self-assemble into capsid-like particles and thus can be used for the formation of the virus particles of the present invention.

Thus, in some embodiments, the viral structural protein is PEG10. As used herein, the term “PEG10” refers to the retrotransposon-derived protein PEG10 that is encoded by the PEG10 gene. In particular, PEG10 has a CCHC-type zinc finger domain containing a sequence characteristic of gag proteins of most retroviruses. An exemplary amino acid sequence for PEG10 is represented by SEQ ID NO:22 (isoform 1) or SEQ ID NO: 23 (isoform 2).

>sp|Q86TG7|PEG10_HUMAN Retrotransposon-derived protein
PEG10 OS = Homo sapiens OX = 9606 GN = PEG10 PE = 1 SV = 2.
SEQ ID NO: 22
MTERRRDELSEEINNLREKVMKQSEENNNLQSQVQKLTEENTTLREQVEPTPEDEDDDIE
LRGAAAAAAPPPPIEEECPEDLPEKFDGNPDMLAPFMAQCQIFMEKSTRDFSVDRVRVCF
VTSMMTGRAARWASAKLERSHYLMHNYPAFMMEMKHVFEDPQRREVAKRKIRRLRQGMGS
VIDYSNAFQMIAQDLDWNEPALIDQYHEGLSDHIQEELSHLEVAKSLSALIGQCIHIERR
LARAAAARKPRSPPRALVLPHIASHHQVDPTEPVGGARMRLTQEEKERRRKLNLCLYCGT
GGHYADNCPAKASKSSPAGKLPGPAVEGPSATGPEIIRSPQDDASSPHLQVMLQIHLPGR
HTLFVRAMIDSGASGNFIDHEYVAQNGIPLRIKDWPILVEAIDGRPIASGPVVHETHDLI
VDLGDHREVLSFDVTQSPFFPVVLGVRWLSTHDPNITWSTRSIVFDSEYCRYHCRMYSPI
PPSLPPPAPQPPLYYPVDGYRVYQPVRYYYVQNVYTPVDEHVYPDHRLVDPHIEMIPGAH
SIPSGHVYSLSEPEMAALRDFVARNVKDGLITPTIAPNGAQVLQVKRGWKLQVSYDCRAP
NNFTIQNQYPRLSIPNLEDQAHLATYTEFVPQIPGYQTYPTYAAYPTYPVGFAWYPVGRD
GQGRSLYVPVMITWNPHWYRQPPVPQYPPPQPPPPPPPPPPPPP
>sp|Q86TG7-2|PEG10 HUMAN Isoform 2 of Retrotransposon-
derived protein PEG10 OS = Homo sapiens OX = 9606 GN = PEG10
SEQ ID NO: 23
MTERRRDELSEEINNLREKVMKQSEENNNLQSQVQKLTEENTTLREQVEPTPEDEDDDIE
LRGAAAAAAPPPPIEEECPEDLPEKFDGNPDMLAPFMAQCQIFMEKSTRDFSVDRVRVCF
VTSMMTGRAARWASAKLERSHYLMHNYPAFMMEMKHVFEDPQRREVAKRKIRRLRQGMGS
VIDYSNAFQMIAQDLDWNEPALIDQYHEGLSDHIQEELSHLEVAKSLSALIGQCIHIERR
LARAAAARKPRSPPRALVLPHIASHHQVDPTEPVGGARMRLTQEEKERRRKLNLCLYCGT
GGHYADNCPAKASKSSPAGNSPAPL

Thus, in some embodiments, the viral structural protein has an amino acid sequence having at least 70% of identity with the amino acid sequence a set forth in SEQ ID NO:22 or SEQ ID NO:23.

Methods for producing virus particles are well known in the art. Typically, vectors for expressing the required viral structural protein and the syncytin-1 fusion protein of the present invention are used for expressing nucleic sequences within the desired packaging cells. Notably, vectors for expressing the required proteins or nucleic acids comprise an open reading frame which is placed under the control of regulatory elements that are functional in the packaging cell wherein their expression is sought. Notably, these vectors comprise, for each protein or nucleic acid to be expressed, an open reading frame which is placed under the control of a suitable promoter sequence, as well as a polyadenylation sequence. As it is well known in the art, a nucleic acid vector is introduced into the packaging cell by any of a variety of techniques (e.g., calcium phosphate co-precipitation, lipofection, electroporation). The viral proteins produced by the packaging cell mediate the insertion of the viral protein(s), the syncytin-1 fusion protein of the present invention and optionally the cargo (e.g. polypeptides or polynucleotides) into the virus-derived particles, which are then released into the culture supernatant. The nucleic acid vectors used may be derived from a retrovirus (e.g., a lentivirus). For instance, retrovirus vectors suitable for producing the virus-derived particles described herein allow (1) transfection of the packaging vectors and envelope vectors into the host cell to form a packaging cell line that produces the virus particles pseudotyped with the syncytin-1 protein of the present invention, and (2) the packaging of the cargo (e.g. Cas protein and optionally also of the CRISPR guide RNA(s)) into the virus-derived particles. Illustratively, a vector for expressing the viral structural protein, e.g. a Gag protein or a Gag-Pro-Pol fusion protein, and optionally also a viral envelope protein, e.g. a VSV-G protein or a BAEV-G protein, may be prepared by the one skilled in the art according to the teachings of Negre et al. (2000, Gene Ther, Vol. 7: 1613-1623) and of Yee et al. (1994, Methods Cell Biol, Vol. 43 PtA: 99-112).

Any suitable permissive or packaging cell known in the art may be employed in the production of the virus-derived particles described herein. Mammalian cells or insect cells are preferred. Examples of cells useful for the production of the virus-derived particles in the practice of the invention include, for example, human cell lines, such as VERO, WI38, MRC5, A549, HEK293, HEK293T, B-50 or any other HeLa cells, HepG2, Saos-2, HuH7, and HT1080 cell lines. Illustrative cell lines for use as packaging cells also include insect cell lines. A number of cell types can be used, which encompasses: a) NIH-3T3 murine cells which are currently widely used as packaging cells producing recombinant retroviruses in clinical use (Takahara et al., Journal of Virology, (June 1992), 66 (6) 3725-32) and b) TK⁻ cell lines have already been described, including NIH-3T3 TK cells (F. Wagner et al., EMBO Journal (1985), Vol. 4 (no 3): 663-666); these cells can be killed when they are cultivated in selective culture media such as HAT. If they are complemented for the kinase thymidine function, for example those from the HSV1-TK virus, they can grow in a selective medium; such lines thus offer the possibility of using the HSV1-TK gene as a selection gene. The gene coding for the thymidine kinase of HSV1 or one of its functional derivatives is also widely used as a transgene as a pro-drug transforming ganciclovir or acyclovir into a drug which is cytotoxic for the cell, and it can thus be applied to selective cell destruction, for example of cancerous cells (see, for example, International patent application WO 95/22617).

A further object of the present invention thus relates to a cell line for producing a virus particle as described herein, comprising i) one or more polynucleotides encoding the structural viral proteins required for forming the said the virus particle, ii) a polynucleotide encoding for the syncytin-1 fusion protein of the present invention and iii) one or more polynucleotide encoding for the cargo(s).

### Cargos:

According to the present invention, the particle of the present invention encapsulates one or more cargos. Typically, the cargo can be of any nature compatible with the encapsulation in particles, such as virus particles.

In some embodiments, the cargo is selected from the group consisting of organic molecules, polymers, polypeptides polynucleotides and small organic compounds having a molecular weight of more than 50 and less than about 2,500 daltons. Cargos are also found among biomolecules including peptides, saccharides, fatty acids, lipids, steroids, purines, pyrimidines, derivatives, structural analogs or combinations thereof.

In some embodiments, cargos include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

In some embodiments, the cargo is a polynucleotide. In some embodiments, the polynucleotide is an RNA or a DNA molecule.

In some embodiments, the polynucleotide is introduced into the target cells of a tissue or an organ and is capable of being expressed under appropriate conditions, or otherwise conferring a beneficial property to the cells. The polynucleotide is thus selected based upon a desired therapeutic outcome. For instance, the polynucleotide encodes for to a polypeptide that confers a beneficial property to the cells or a desired therapeutic outcome. Examples of polynucleotides of interest include but are not limited to those encoding for a polypeptide selected from the group consisting of protective polypeptides (e.g., neuroprotective polypeptides such as GDNF, CNTF, NT4, NGF, and NTN); anti-angiogenic polypeptides (e.g., a soluble vascular endothelial growth factor (VEGF) receptor; a VEGF-binding antibody; a VEGF-binding antibody fragment (e.g., a single chain anti-VEGF antibody); and anti-apoptotic polypeptides (e.g., Bcl-2, Bcl-Xl); and the like.

In some embodiments, the polynucleotide encodes for an antigen. As used herein, the term “antigen” has its general meaning in the art and generally refers to a substance or fragment thereof that is recognized and selectively bound by an antibody or by a T cell antigen receptor, resulting in induction of an immune response. Antigens according to the invention are typically, although not exclusively, peptides and proteins. An antigen in the context of the invention can comprise any subunit, fragment, or epitope of any proteinaceous molecule, including a protein or peptide of viral, bacterial, parasitic, fungal, protozoan, prion, cellular, or extracellular origin, which ideally provokes an immune response in mammal, preferably leading to protective immunity. In some embodiments, the antigen is a tumor antigen. In particular, the antigen can be a peptide isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae (e.g., Norovirus (also known as “Norwalk-like virus”)), Capillovirus, Carlavirus, Caulimovirus, Circoviridae, Closterovirus, Comoviridae, Coronaviridae (e.g., Coronavirus, such as severe acute respiratory syndrome (SARS) virus, or SARS-CoV-2), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae (e.g., Hepatitis B virus or Hepatitis C virus), Herpesviridae (e.g., Human herpesvirus (HSV) 1, 2, 3, 4, 5, and 6, Cytomegalovirus, and Epstein-Barr Virus (EBV)), Hypoviridae, Iridoviridae, Leviviridae, Lipothrixviridae, Microviridae, Orthomyxoviridae (e.g., Influenzavirus A and B), Papovaviridae, Papillomaviridae (e.g., human papillomavirus (HPV)), Paramyxoviridae (e.g., measles, mumps, and human respiratory syncytial virus (RSV)), Parvoviridae, Picornaviridae (e.g., poliovirus, rhinovirus, hepatovirus, and aphthovirus (e.g., foot and mouth disease virus)), Poxviridae (e.g., vaccinia virus), Reoviridae (e.g., rotavirus), Retroviridae (e.g., lentivirus, such as human immunodeficiency virus (HIV) 1 and HIV 2), Rhabdoviridae, and Totiviridae.

In some embodiments, the polynucleotide of the present invention is an RNA molecule, in particular a messenger RNA (mRNA). In some embodiments, the particle of the present invention encapsuled one or more RNA molecules capable of inducing: i) transfer of one or more endogenous or exogenous coding sequences of interest of the target cell, ii) transfer of one or more non-coding RNAs such as RNAs capable of inducing an effect on genetic expression, for example by means of shRNA, miRNA, sgRNA, LncRNA or circRNA, iii) transfer of cellular RNAs, of the messenger RNA type or others (miRNA etc.), subgenomic replicons of RNA viruses (HCV, etc.) or of complete genomes of RNA viruses, iv) simultaneous expression of endogenous or exogenous coding or non-coding sequences of the target cell, or vi) participation in modification of the genome of the target cell by genome engineering systems, for example the CRISPR system.

In some embodiments, the RNA molecule encapsuled in the virus particle of the present invention comprises at least one encapsidation sequence. By “encapsidation sequence” is meant an RNA motif (sequence and three-dimensional structure) recognized specifically by an RNA-binding domain as above described.

In some embodiments, the polynucleotide is an antisense or siRNA sequence that acts to reduce expression of a targeted sequence. Antisense or siRNA nucleic acids are designed to specifically bind to RNA, resulting in the formation of RNA-DNA or RNA-RNA hybrids, with an arrest of DNA replication, reverse transcription or messenger RNA translation. Gene expression is reduced through various mechanisms. Antisense nucleic acids based on a selected nucleic acid sequence can interfere with expression of the corresponding gene. Antisense oligodeoxynucleotides (ODN), include synthetic ODN having chemical modifications from native nucleic acids, or nucleic acid constructs that express such anti-sense molecules as RNA. One or a combination of antisense molecules may be administered, where a combination may comprise multiple different sequences. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like.

Also of interest are RNAi agents. RNAi agents are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in some embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in some embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides.

In some embodiments, the cargo is a polynucleotide that encodes for an endonuclease, a base-editing enzyme, an epigenome editor or a prime editor as described herein after.

In some embodiments, the cargo is a polypeptide. Polypeptides of interest include biologically active proteins, e.g. transcription factors, proteins involved in signaling pathways, cytokines, chemokines, toxins, and the like. Such polypeptides may include proteins not found in the target cell, proteins from different species or cloned versions of proteins found in the target cell. Preferred target proteins of the invention will be proteins with the same status as that found in the target cell expressed in such a way that post-translational modification is the same as that found in the target cell. Such modification includes glycosylation or lipid modification addition of coenzyme groups or formation of quaternary structure. Most preferred will be wild type proteins corresponding to proteins found in mutated form or absent in the target cell. In some embodiment, the polypeptide is a membrane protein or a non-membrane protein. Non-limiting examples of membrane proteins include ion channels, receptor tyrosine kinases such as the PDGF-receptor and the SCF-R receptor (Stem Cell Factor Receptor, or c-kit, or CD 117), G-protein linked receptors such as adrenoreceptors. Non-limiting examples of non-membrane proteins include cytosolic proteins such as actin, Ras, ERK1/2 and nuclear proteins such as steroid receptors, histone proteins, or transcriptional factors.

In some embodiments, the cargo is an endonuclease that provides for site-specific knock-down of gene function, e.g., where the endonuclease knocks out an allele associated with a genetic disease. For example, where a dominant allele encodes a defective copy of a gene that, when wild-type, is a retinal structural protein and/or provides for normal retinal function, a site-specific endonuclease can be targeted to the defective allele and knock out the defective allele. In addition to knocking out a defective allele, a site-specific nuclease can also be used to stimulate homologous recombination with a donor DNA that encodes a functional copy of the protein encoded by the defective allele. Thus, e.g., the method of the invention can be used to deliver both a site-specific endonuclease that knocks out a defective allele, and can be used to deliver a functional copy of the defective allele, resulting in repair of the defective allele, thereby providing for production of a functional protein.

In some embodiments, the DNA targeting endonuclease is a Transcription Activator-Like Effector Nuclease (TALEN). TALENs are produced artificially by fusing a TAL effector (“TALE”) DNA binding domain, e.g., one or more TALEs, e.g., 1, 2, 3, 4, 5, 6, 7, 8, 9 or 10 TALEs to a DNA-modifying domain, e.g., a FokI nuclease domain. Transcription activator-like effects (TALEs) can be engineered to bind any desired DNA sequence (Zhang (2011), Nature Biotech. 29: 149-153). By combining an engineered TALE with a DNA cleavage domain, a restriction enzyme can be produced which is specific to any desired DNA sequence. These can then be introduced into a cell, wherein they can be used for genome editing (Boch (2011) Nature Biotech. 29: 135-6; and Boch et al. (2009) Science 326: 1509-12; Moscou et al. (2009) Science 326: 3501). TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence (Zhang (2011), Nature Biotech. 29: 149-153). To produce a TALEN, a TALE protein is fused to a nuclease (N), e.g., a wild-type or mutated FokI endonuclease. Several mutations to FokI have been made for its use in TALENs; these, for example, improve cleavage specificity or activity (Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96). The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity (Miller et al. (2011) Nature Biotech. 29: 143-8). TALEN can be used inside a cell to produce a double-strand break in a target nucleic acid, e.g., a site within a gene. A mutation can be introduced at the break site if the repair mechanisms improperly repair the break via non-homologous end joining (Huertas, P., Nat. Struct. Mol. Biol. (2010) 17: 11-16). For example, improper repair may introduce a frame shift mutation. Alternatively, foreign DNA can be introduced into the cell along with the TALEN; depending on the sequences of the foreign DNA and chromosomal sequence, this process can be used to modify a target gene via the homologous direct repair pathway, e.g., correct a defect in the target gene, thus causing expression of a repaired target gene, or e.g., introduce such a defect into a wt gene, thus decreasing expression of a target gene.

In some embodiments, the DNA targeting endonuclease is a Zinc-Finger Nuclease (ZFN). Like a TALEN, a ZFN comprises a DNA-modifying domain, e.g., a nuclease domain, e.g., a FokI nuclease domain (or derivative thereof) fused to a DNA-binding domain. In the case of a ZFN, the DNA-binding domain comprises one or more zinc fingers, e.g., 1, 2, 3, 4, 5, 6, 7, 8, 9 or 10 zinc fingers (Carroll et al. (2011) Genetics Society of America 188: 773-782; and Kim et al. (1996) Proc. Natl. Acad. Sci. USA 93: 1156-1160). A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art (Sera (2002), Biochemistry, 41:7074-7081; Liu (2008) Bioinformatics, 24:1850-1857). A ZFN using a FokI nuclease domain or other dimeric nuclease domain functions as a dimer. Thus, a pair of ZFNs are required to target non-palindromic DNA sites. The two individual ZFNs must bind opposite strands of the DNA with their nucleases properly spaced apart (Bitinaite et al. (1998) Proc. Natl. Acad. Sci. USA 95: 10570-5). Also like a TALEN, a ZFN can create a DSB in the DNA, which can create a frame-shift mutation if improperly repaired, e.g., via non-homologous end joining, leading to a decrease in the expression of a target gene in a cell.

In some embodiments, the DNA targeting endonuclease is a CRISPR-associated endonuclease. In bacteria the CRISPR/Cas loci encode RNA-guided adaptive immune systems against mobile genetic elements (viruses, transposable elements and conjugative plasmids). Three types (I-VI) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activated small RNA (tracrRNA) that serves as a guide for ribonuclease Ill-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3^{rd} or the 4^{th} nucleotide from PAM). The crRNA and tracrRNA can be expressed separately or engineered into an artificial fusion small guide RNA (sgRNA) via a synthetic stem loop to mimic the natural crRNA/tracrRNA duplex. Such sgRNA, like shRNA, can be synthesized or in vitro transcribed for direct RNA transfection or expressed from U6 or H1-promoted RNA expression vector.

In some embodiments, the CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomonas aeruginosa, Escherichia coli, or other sequenced bacteria genomes and archaea, or other prokaryotic microorganisms. Alternatively, the wild type Streptococcus pyogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as pX330, pX260 or pMJ920 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of pX330, pX260 or pMJ920 (Addgene, Cambridge, MA).

In some embodiments, the CRISPR-associated endonuclease is a Cpf1 nuclease. As used herein, the term “Cpf1 protein” to a Cpf1 wild-type protein derived from Type V CRISPR-Cpf1 systems, modifications of Cpf1 proteins, variants of Cpf1 proteins, Cpf1 orthologs, and combinations thereof. The cpf1 gene encodes a protein, Cpf1, that has a RuvC-like nuclease domain that is homologous to the respective domain of Cas9, but lacks the HNH nuclease domain that is present in Cas9 proteins. Type V systems have been identified in several bacteria, including Parcubacteria bacterium GWC2011_GWC2_44_17 (PbCpf1), Lachnospiraceae bacterium MC2017 (Lb3 Cpf1), Butyrivibrio proteoclasticus (BpCpf1), Peregrinibacteria bacterium GW2011_GWA 33_10 (PeCpf1), Acidaminococcus spp. BV3L6 (AsCpf1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculi 237 (MbCpf1), Smithella spp. SC_K08D17 (SsCpf1), Leptospira inadai (LiCpf1), Lachnospiraceae bacterium MA2020 (Lb2Cpf1), Franciscella novicida U112 (FnCpf1), Candidatus methanoplasma termitum (CMtCpf1), and Eubacterium eligens (EeCpf1). Recently it has been demonstrated that Cpf1 also has RNase activity and it is responsible for pre-crRNA processing (Fonfara, I., et al., “The CRISPR-associated DNA-cleaving enzyme Cpf1 also processes precursor CRISPR RNA,” Nature 28; 532(7600):517-21 (2016)).

In some embodiments, the cargo is a base-editing enzyme. As used herein, the term “base-editing enzyme” refers to fusion protein comprising a defective CRISPR/Cas nuclease linked to a deaminase polypeptide. The term is also known as “base-editor”. As used herein, the term “deaminase” refers to an enzyme that catalyses a deamination reaction. The term “deamination”, as used herein, refers to the removal of an amine group from one molecule. In some embodiments, the deaminase is a cytidine deaminase, catalysing the hydrolytic deamination of cytidine or deoxycytidine to uracil or deoxyuracil, respectively. In some embodiments, the deaminase is an adenosine deaminase, catalysing the hydrolytic deamination of adenosine to inosine, which is treated like guanosine by the cell, creating an A to G (or T to C) change. Two classes of base-editing enzymes—cytosine base-editing enzymes (CBEs) and adenine base-editing enzymes (ABEs)—can be used to generate single base pair edits without double stranded breaks. Typically, cytosine base-editing enzymes are created by fusing the defective CRISPR/Cas nuclease to a deaminase.

In some embodiments, the base-editing enzyme comprises a defective CRISPR/Cas nuclease. The sequence recognition mechanism is the same as for the non-defective CRISPR/Cas nuclease. Typically, the defective CRISPR/Cas nuclease of the invention comprises at least one RNA binding domain. The RNA binding domain interacts with a guide RNA molecule as defined hereinafter. However, the defective CRISPR/Cas nuclease of the invention is a modified version with no nuclease activity. Accordingly, the defective CRISPR/Cas nuclease specifically recognizes the guide RNA molecule and thus guides the base-editing enzyme to its target DNA sequence. In some embodiments, the CRISPR/Cas nuclease consists of a mutant CRISPR/Cas nuclease i.e. a protein having one or more point mutations, insertions, deletions, truncations, a fusion protein, or a combination thereof. In some embodiments, the mutant has the RNA-guided DNA binding activity, but lacks one or both of its nuclease active sites. In some embodiments, the CRISPR/Cas nuclease of the present invention is nickase and more particularly a Cas9 nickase i.e. the Cas9 from S. pyogenes having one mutation selected from the group consisting of DOA and H840A.

The second component of the base-editing enzyme herein disclosed comprises a non-nuclease DNA modifying enzyme that is a deaminase.

In some embodiments, the deaminase is a cytidine deaminase. In some embodiments, the deaminase is an apolipoprotein B mRNA-editing complex (APOBEC) family deaminase. In some embodiments, the deaminase is an APOBEC1 family deaminase. In some embodiments, the deaminase is an activation-induced cytidine deaminase (AID). In some embodiments, the deaminase is an ACF1/ASE deaminase.

In some embodiments, the deaminase is selected from the group consisting of AID: activation induced cytidine deaminase, APOBEC1: apolipoprotein B mRNA editing enzyme, catalytic polypeptide-like 1, APOBEC3A: apolipoprotein B mRNA editing enzyme, catalytic polypeptide-like 3A, APOBEC3B: apolipoprotein B mRNA editing enzyme, catalytic polypeptide-like 3B, APOBEC3C: apolipoprotein B mRNA editing enzyme, catalytic polypeptide-like 3C, APOBEC3D: apolipoprotein B mRNA editing enzyme, catalytic polypeptide-like 3D, APOBEC3F: apolipoprotein B mRNA editing enzyme, catalytic polypeptide-like 3F, APOBEC3G: apolipoprotein B mRNA editing enzyme, catalytic polypeptide-like 3G, APOBEC3H: apolipoprotein B mRNA editing enzyme, catalytic polypeptide-like 3H, ADA: adenosine deaminase, ADAR1: adenosine deaminase acting on RNA 1, Dnmt1: DNA (cytosine-5-)-methyltransferase 1, Dnmt3a: DNA (cytosine-5-)-methyltransferase 3 alpha, Dnmt3b: DNA (cytosine-5-)-methyltransferase 3 beta and Tet1: methylcytosine dioxygenase.

In some embodiments, the deaminase derives from the Activation Induced cytidine Deaminase (AID). AID is a cytidine deaminase that can catalyze the reaction of deamination of cytosine in the context of DNA or RNA. When brought to the targeted site, AID changes a C base to U base. In dividing cells, this could lead to a C to T point mutation. Alternatively, the change of C to U could trigger cellular DNA repair pathways, mainly excision repair pathway, which will remove the mismatching U-G base-pair, and replace with a T-A, A-T, C-G, or G-C pair. As a result, a point mutation would be generated at the target C-G site. In some embodiments, the DNA modifying enzyme is AID*Δ that is an AID mutant with increased SHM activity whose Nuclear Export Signal (NES) has been removed (Hess G T, Fresard L, Han K, Lee C H, Li A, Cimprich K A, Montgomery S B, Bassik M C: Directed evolution using dCas9-targeted somatic hypermutation in mammalian cells. Nat Methods 2016, 13(12):1036-1042).

In some embodiments, the deaminase is an adenosine deaminase. In some embodiments, the deaminase is an ADAT family deaminase. In some embodiments, the adenosine deaminase variant is a TadA deaminase. In some embodiments, the adenosine deaminase variant is a Staphylococcus aureus TadA, a Bacillus subtilis TadA, a Salmonella typhimurium TadA, a Shewanella putrefaciens TadA, a Haemophilus influenzae F3031 TadA, a Caulobacter crescentus TadA, or a Geobacter sulfurreducens TadA, or a fragment thereof. In some embodiments, the TadA deaminase is an E. coli TadA deaminase (ecTadA). In some embodiments, the TadA deaminase is a truncated E. coli TadA deaminase. In some embodiments, the TadA deaminase is TadA*7.10. In some embodiments, the TadA deaminase is a TadA*8 variant. For example, deaminase are described in International PCT Application WO2018/027078, WO2017/070632, WO/2020/168132, WO/2021/050571 each of which is incorporated herein by reference for its entirety. Also, see Komor, A. C., et al., “Programmable editing of a target base in genomic DNA without double-stranded DNA cleavage” Nature 533, 420-424 (2016); Gaudelli, N. M., et al., “Programmable base editing of A⋅T to G⋅C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017)), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of which are hereby incorporated by reference.

In some embodiments, the cargo is an epigenome editing effector (“EpiEditor”) that enables to activate and repress endogenous gene expression and can provide graded control over gene regulation (Nakamura, M, Gao, Y., Dominguez, A. A. et al. CRISPR technologies for precise epigenome editing. Nat Cell Biol 23, 11-22 (2021)). Recruitment of epigenome editing effector domains typically involves CRISPR/Cas systems that allow site-specific control over modifications to DNA, histones, and chromatin architecture.

In some embodiments, the cargo is a prime editor that consists of a fusion protein wherein a catalytically impaired Cas9 endonuclease is fused to an engineered reverse transcriptase enzyme. By complexing a prime editing guide RNA (pegRNA), the prime editor is capable of identifying the target site and providing the new genetic information to replace the target DNA nucleotides. It mediates targeted insertions, deletions, and base-to-base conversions without the need for double strand breaks (DSBs) or donor DNA templates (Anzalone, Andrew V.; Randolph, Peyton B.; Davis, Jessie R.; Sousa, Alexander A.; Koblan, Luke W.; Levy, Jonathan M.; Chen, Peter J.; Wilson, Christopher; Newby, Gregory A.; Raguram, Aditya; Liu, David R. (21 Oct. 2019). “Search-and-replace genome editing without double-strand breaks or donor DNA”. Nature. 576 (7785): 149-157.).

In some embodiments, the particle of the present invention encapsulates i) a polypeptide (or a polynucleotide encoding thereof) selected from the group consisting of CRISPR-associated endonucleases, base editing enzymes, epigenome editing factors and primer editors and ii) one or more guide RNA molecules.

As used herein, the term “guide RNA molecule” generally refers to an RNA molecule (or a group of RNA molecules collectively) that can bind to a Cas9 protein and target the Cas9 protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9 related polypeptide. These two segments can be located in the same RNA molecule or in two or more separate RNA molecules. When the two segments are in separate RNA molecules, the molecule comprising the DNA-targeting guide segment is sometimes referred to as the CRISPR RNA (crRNA), while the molecule comprising the protein-binding segment is referred to as the trans-activating RNA (tracrRNA).

In some embodiments, the cargo polypeptide is fused to the viral structural protein (e.g. the GAG or PEG10 protein) either directly or via a linker. For instance, in some embodiments, the nuclease (e.g. Cas9) is fused to the viral structural protein directly either directly or via a linker.

In some embodiments, the cargo polypeptide (e.g. the nuclease such as Cas9) and the viral structural protein (e.g. the GAG or PEG10 protein) form a dimer. The means by which the viral structural protein and the cargo polypeptide form a dimer is not particularly limited. In some embodiments, the viral structural protein (e.g. the GAG or PEG10 protein) and the cargo polypeptide (e.g. the nuclease such as Cas9) are fused either directly or via a linker to respective domains that are capable of dimerization in presence of a compound. For instance, it is possible to use a system in which FK506-binding protein (“FKBP12 domain”) and FKBP12-rapamycin-associated protein 1, FRAP1 fragment (“FRB domain”) form a heterodimer in the presence of rapamycin. Thus, in some embodiments, the viral structural protein (e.g. GAG of PEG10) is fused to the FRB domain and the cargo polypeptide (e.g. the nuclease such as Cas9) is fused to the FKBP12 domain (or vice-versa), it is possible to dimerize the FKBP12 domain and the FRB domain in presence of rapamycin during the production of the virus particles. Alternatively, it is possible to use a system in which GAI (Gibberellin insensitive) and GID1 (Gibberellin insensitive dwarf1) form a heterodimer in the presence of gibberellin or GA3-AM (for example, see Miyamoto T., et al., Rapid and Orthogonal Logic Gating with a Gibberellin-induced Dimerization System, Nat Chem Biol., 8 (5), 465-470, 2012), a system in which PyL (PYR1-like, consisting of the 33rd to 209th amino acids) and ABI1 (consisting of the 126th to 423rd amino acids) form a heterodimer in the presence of S-(+)-abscisic acid (ABA) (for example, see, Liang F. S., et al., Engineering the ABA plant stress pathway for regulation of induced proximity, Sci Signal., 4 (164), rs2, 2011), and the like.

Other cargos of interest include detectable markers, e.g. luciferase, luciferin, green fluorescent proteins, fluorochromes, e.g. FITC, etc., and the like. Detectable markers may also include imaging entities, e.g. metallic nanoparticles such as gold, platinum, silver, etc., which may be provided as nanoparticles, usually nanoparticles of less than 10 nm, less than about 5 nm, etc.

### Therapeutic Uses:

The present invention provides particles compositions and kits suitable for use in therapy (in vivo or ex vivo). According to the present invention, the therapeutical effects are brought by the one or more cargo(s) that is(are) encapsulated in the particles of the present invention. For instance, the particles as well as the compositions comprising them may be used for gene therapy or vaccine purposes.

Thus a further object of the present invention relates to a method of therapy in a subject in need thereof comprising administering to the subject a therapeutically amount of the particle of the present invention.

Types of diseases and disorders that can be treated by methods of the present invention include, but are not limited to, retinal diseases such as age-related macular degeneration; diabetic retinopathy; infectious diseases e.g., HIV pandemic flu, category 1 and 2 agents of biowarfare, or any new emerging viral infection; autoimmune diseases; cancer; multiple myeloma; diabetes; systemic lupus erythematosus (SLE); hepatitis C; multiple sclerosis; Alzheimer's disease; parkinson's disease; amyotrophic lateral sclerosis (ALS), huntington's disease; epilepsy; chronic obstructive pulmonary disease (COPD); joint inflammation, arthritis; myocardial infarction (MI); congestive heart failure (CHF); hemophilia A; or hemophilia B.

Infectious diseases that can be treated or prevented by the methods of the present invention are caused by infectious agents including, but not limited to, viruses, bacteria, fungi, protozoa, helminths, and parasites. The invention is not limited to treating or preventing infectious diseases caused by intracellular pathogens. Many medically relevant microorganisms have been described extensively in the literature, e.g., see C. G. A Thomas, Medical Microbiology, Bailliere Tindall, Great Britain 1983, the entire contents of which are hereby incorporated herein by reference.

Types of cancers that can be treated or prevented by the methods of the present invention include, but are not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

In some embodiments, when the particles of the present invention are designed for targeting a hematopoietic cell, the method of therapy herein disclosed is particularly suitable for the treatment of β-hemoglobinopathies.

As used herein, the term “β-hemoglobinopathy” has its general meaning in the art and refers to any defect in the structure or function of any hemoglobin of an individual, and includes defects in the primary, secondary, tertiary or quaternary structure of hemoglobin caused by any mutation, such as deletion mutations or substitution mutations in the coding regions of the HBB gene, or mutations in, or deletions of, the promoters or enhancers of such gene that cause a reduction in the amount of hemoglobin produced as compared to a normal or standard condition.

In some embodiments, the particles of the present invention are particularly suitable for the treatment of sickle cell disease.

As used herein, the term “sickle cell disease” has its general meaning in the art and refers to a group of autosomal recessive genetic blood disorders, which results from mutations in a globin gene and which is characterized by red blood cells that assume an abnormal, rigid, sickle shape. They are defined by the presence of βS-globin gene coding for a β-globin chain variant in which glutamic acid is substituted by valine at amino acid position 6 of the peptide: incorporation of the βS-globin in the Hb tetramers (HbS, sickle Hb) leads to Hb polymerization and to a clinical phenotype. The term includes sickle cell anemia (HbSS), sickle-hemoglobin C disease (HbSC), sickle beta-plus-thalassaemia (HbS/β+), or sickle beta-zerothalassaemia (HbS/β0).

In some embodiments, the particles of the present invention are particularly suitable for the treatment of β-thalassemia.

As used herein, the term “β-thalassemia” refers to a hemoglobinopathy that results from an altered ratio of α-globin to β-like globin polypeptide chains resulting in the underproduction of normal hemoglobin tetrameric proteins and the precipitation of free, unpaired α-globin chains. Compositions as described herein encompass pharmaceutical compositions that are used for the purpose of performing a method of therapy in subject in need thereof, which includes non-human mammals and human individuals in need thereof. Compositions of the invention may be formulated for delivery to animals for veterinary purposes (e.g., livestock such as cattle, pigs, etc), and other non-human mammalian subjects, as well as to human subjects. For instance, the particles may be formulated with a physiologically acceptable carrier for use in gene transfer and gene therapy applications. In some embodiments, the said composition further comprises one or more transduction helper compounds. The transduction helper compounds are preferably selected in a group comprising cationic polymers, as described notably by Zuris et al. (2015, Nat Biotechnol, Vol. 33 (no 1): 73-80). The transduction helper compound may be selected in a group comprising polybrene (that may be also termed hexadimethrine bromide), protamine sulfate, 12-myristate 13-acetate (also termed phorbol myristate acetate or PMA, as described by Johnston et al., 2014, Gene Ther, Vol. 21(12): 1008-1020), vectofusin (as described by Fenard et al., 2013, Molecular Therapy Nucleic Acids, Vol. 2: e90), poloxamer P338 (as described by Anastasov et al., 2016, Lentiviral vectors and exosomes as gene and protein delivery tools, in Methods in Molecular Biology, Vol. 1448: 49-61), RetroNectin® Reagent (commercialized by Clontech Laboratories Inc.), Viral Plus® transduction enhancer (commercialized by Applied Biological Materials Inc.), TransPlus® Virus Transduction Enhancer (commercialized by Clinisciences), Lentiboost® (commercialized by Sirion Biotech), or ExpressMag® Transduction System (commercialized by Sigma-Aldrich). As shown in the examples herein, the said cationic transduction helper compound may consist of polybrene. The particles may be formulated in a conventional manner using one or more physiologically acceptable carriers or excipients. The particles may be formulated for parenteral administration by injection, e.g., by bolus injection or continuous infusion. Formulations for injection may be presented in unit dosage form, e.g., in ampoules or in multi-dose containers, with an added preservative. The particles compositions may take such forms as suspensions, solutions or emulsions in oily or aqueous vehicles, and may contain formulation agents such as suspending, stabilizing and/or dispersing agents. Liquid preparations of the particles compositions may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts. Alternatively, the compositions may be in powder form for constitution with a suitable vehicle, e.g., sterile pyrogen-free water, before use.

The particles compositions of the invention may be administered to a subject at therapeutically effective doses to provide the therapeutic effects. In some embodiments, an amount of particles composition of the invention is administered at a dose unit that is in the range of about 0.1-5 micrograms (g)/kilogram (kg). To this end, the particles composition of the invention may be formulated in doses in the range of about 7 mg to about 350 mg to treat to treat an average subject of 70 kg in body weight. The amount of particles composition of the invention that may be administered may be selected in a group comprising 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg or 5.0 mg/kg. The dose of particles in a unit dosage of the composition may be selected in a group comprising 7 mg, 8 mg, 9 mg, 10 mg, 20 mg, 25 mg, 30 mg, 35 mg, 40 mg, 45 mg, 50 mg, 55 mg, 60 mg, 65 mg, 70 mg, 75 mg, 80 mg, 85 mg 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, or 750 mg, especially for treating an average subject of 70 kg in body weight. These doses can be given once or repeatedly, such as daily, every other day, weekly, biweekly, or monthly. In some embodiments, a virus-like particles composition may be administered to a subject in one dose, or in two doses, or in three doses, or in four doses, or in five doses, or in six doses or more. The interval between dosages may be determined based the practitioner's determination that there is a need thereof.

The particles compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. In some embodiments, the particles composition may be in liquid or solid (e.g. lyophilized) form.

Administration of the particles to a human subject or an animal in need thereof can be by any means known in the art for administering virus vectors. Exemplary modes of administration include rectal, transmucosal, topical, transdermal, inhalation, parenteral (e.g., intravenous, subcutaneous, intradermal, intramuscular, and intraarticular) administration, and the like, as well as direct tissue or organ injection, alternatively, intrathecal, direct intramuscular, intraventricular, intravenous, intraperitoneal, intranasal, or intraocular injections. Injectables can be prepared in conventional forms, either as liquid solutions or suspensions, solid forms suitable for solution or suspension in liquid prior to injection, or as emulsions. Alternatively, one may administer the virus in a local rather than systemic manner, for example, in a depot or sustained-release formulation.

The invention will be further illustrated by the following figures and examples. However, these examples and figures should not be interpreted in any way as limiting the scope of the present invention.

### FIGURES

FIG. 1: (A) Design of viral particles pseudotyped with SYN fused to a ligand. To gain specificity for CD117⁺ or CD133⁺ cells, a scFv antibody fragment against CD133 or the natural ligand of CD117 (stem cell factor (SCF)) were inserted between the signal sequence (SS) and the protein sequence of SYN. We inserted a GGGS flexible linker between the ligand and SYN. (B) CB HSPCs were transfected with lentiviral particles pseudotyped with different ratios of SYN fused to a ligand to WT SYN. Flow cytometry analysis of GFP-expression in CB HSPCs 48 h after transduction. Different ratios were obtaining by transfecting different stoichiometric ratios of a plasmid encoding SYN fused to a ligand (SCF or scFvCD133) to a plasmid expressing WT SYN. A VSV-G pseudotyped virus were used as control. We plotted the fold change compared to SYN WT LV. All LVs expressed GFP under the control of the phosphoglycerate kinase (PGK) promoter.

FIG. 2: (A) Design of viral particles pseudotyped with short mutant of SYN (SYN480) fused to a ligand. (B) Physical titers of LVs were measured by p24 ELISA and expresses as mean±standard deviation (n=3 for SYN480, scFv-SYN480 and SCF-SYN 480 LVs; n=2 for VSV-G). (C) Sorting strategy of CB HSPCs based on either CD117 or CD133 expression. CB HSPCs with low or high CD133 or CD117 cells were FACS-sorted. (D) Different CB HSPCs populations (CD133^{low}, CD133^{high}, CD117^{low} or CD117^{high}) were transduced with equal amounts of LVs pseudotyped with VSV-G, SYN480 or SYN480 fused to the ligand. Flow cytometry analysis of GFP-expression in CB HSPCs was performed 48 h after transduction. Quantifications are relative to the results observed in the population transduced with the LV pseudotyped with SYN480.

FIG. 3: (A) FACS analysis of CD133 and CD117 expression of HEK 293T cells. (B) HEK 293T cells were transduced with different volumes of LV (10, 5 and 1 μl) with different pseudotypes, either VSVG, SYN480, scFvCD133-SYN480 or SCF-SYN480. Flow cytometry analysis of GFP expression in HEK 293T cells 48 h after transduction. (C) Quantification of GFP⁺ HEK 293T cells after transduction with LV pseudotyped with different envelopes.

FIG. 4: (A) CD117^{lo} and CD117^{hi} cells were transduced with LVs pseudotyped with VSVG, SYN480 or different ratio of SYN480 and SCF-SYN480 (33%, 67% and 100%) produced in HEK 293T cells transfected with different amounts of envelope plasmids (6 g, 12 g and 18 μg). Flow cytometry analysis of GFP expression in CB HSPCs was performed 48 h after transduction. (B) Quantifications are relative to the results observed in the population transduced with LV pseudotyped with SYN480. Data are expressed as mean±SD (n=3 biologically independent experiments). (C) Vector Copy Number (VCN) was analyzed in transduced cells 13 days after transduction. Data are expressed as mean±SD (n=3 biologically independent experiments).

FIG. 5: (A) Design of viral particles pseudotyped with short mutant of SYN480 fused to a ligand targeting T cells. To gain specificity for CD4⁺ or CD8⁺ T cells, a DARPin against CD4 or an scFv antibody fragment against CD8 were inserted between the signal sequence (SS) and the protein sequence of SYN. (B) Flow cytometry analysis post-selection to analyze the purity of CD4⁺ and CD8⁺ T cells, respectively (C) CD4⁺ and CD8⁺ T cells were transduced with LVs pseudotyped with VSVG, SYN480 or different ratio of SYN480 and SYN480 fused to the proper ligand (33%, 67% and 100%). LVs were produced in HEK 293T cells transfected with different amounts of envelope plasmids (6 μg, 12 μg and 18 μg). Flow cytometry analysis of GFP expression in T cells were performed 7 days after transduction. (D) Frequency of GFP⁺ cells observed one week after transduction. Data are expressed as mean±SD (n=4 biologically independent experiments). (E) Quantifications are relative to the results observed in the population transduced with LV pseudotyped with SYN480. Data are expressed as mean±SD (n=4 biologically independent experiments).

FIG. 6: (A) Design of viral particles pseudotyped with short mutant of SYN480 fused to a ligand to target IA2+ cells. To gain specificity for IA2+(also known as PTPRN) cells, an scFv antibody fragment against TA2 was inserted between the signal sequence (SS) and the protein sequence of SYN. (B) Flow cytometry analysis assessing IA2 expression in HEK 293T cells and HCT 116 cells. (C) HEK 293T cells and HCT 116 cells were transduced with LV pseudotyped with SYN or 33% of scFv-IA2-SYN480. LVs were produced in HEK 293T cells transfected with different amounts of envelope plasmids (6 μg, 12 μg and 18 g). Flow cytometry analysis of GFP expression in HEK 293T cells and HCT 116 cells were performed 48 hours after transduction. (D) Quantifications are relative to the results observed in the population transduced with LV pseudotyped with SYN480. Data are expressed as mean±SD (n=3 biologically independent experiments). (E) Vector Copy Number (VCN) was analyzed 7 days after transduction. Data are expressed as mean±SD (n=3 biologically independent experiments).

FIG. 7: (A) Design of viral particles pseudotyped with short mutant of SYN480 fused to a ligand to target Glp1R⁺ cells. To gain specificity for Glp1R⁺ cells, the natural ligand of Glp1R (GLP1) was inserted between the signal sequence (SS) and the protein sequence of SYN. To generate a Glp1R⁺ cells, Glp1R was overexpressed by transducing cells with an LV construct containing the Glp1R and the Hygromycin resistance (Hygro) genes. Stably transduced cells were selected using Hygromycin. (B) Flow cytometry analysis assessing Glp1R expression in HEK 293T cells and HCT 116 cells. (C) HEK 293T cells and HEK 293T Glp1R⁺ cells were transduced with LVs pseudotyped with SYN480 or 33% GLP1-SYN480. LVs were produced in HEK 293T cells transfected with different amounts of envelope plasmids (6 μg, 12 g and 18 μg). Flow cytometry analysis of GFP expression in HEK 293T cells and HEK 293T Glp1R⁺ cells were performed 48 hours after transduction. Data are expressed as mean±SD (n=3 biologically independent experiments). (D) Quantifications are relative to the results observed in the population transduced with LV pseudotyped with SYN480 in HEK 293T Glp1R⁺ cells and HEK 293T Glp1R⁺ cells and HCT 116 Glp1R⁺ cells and HCT 116 Glp1R⁺ cells. Data are expressed as mean±SD (n=3 biologically independent experiments).

TABLE 1
Titers of SYN480 and ligand-SYN480 pseudotyped LVs.
Physical titers were measured by p24 ELISA. Data are
expressed as mean ± SD (n = 3 independent batches).

| Vector | |
|---|---|
| Type | ng p24/mL |
| SYN480 6 ug | 2.34E+06 ± 2.25 |
| 33% Glp1L-SYN480 6 ug | 1.39E+06 ± 0.22 |
| 33% scFVIA2-SYN480 6 ug | 1.39E+06 ± 0.11 |
| 33% SCF-SYN480 6 ug | 2.55E+06 ± 1.77 |
| 33% CD4-SYN480 6 ug | 1.28E+06 ± 1.24 |
| 33% CD8-SYN480 6 ug | 1.40E+06 ± 1.59 |
| SYN480 12 ug | 1.51E+06 ± 0.6 |
| 33% Glp1L-SYN480 12 ug | 9.66E+05 ± 0.1 |
| 33% scFVIA2-SYN480 12 ug | 2.01E+06 ± 1.38 |
| 33% SCF-SYN480 12 ug | 2.06E+06 ± 1.68 |
| 33% CD4-SYN480 12 ug | 5.77E+05 ± 0.62 |
| 33% CD8-SYN480 12 ug | 6.01E+05 ± 0.66 |
| SYN480 18 ug | 4.71E+05 ± 0.24 |
| 33% Glp1L-SYN480 18 ug | 5.79E+05 ± 0.20 |
| 33% scFvIA2-SYN480 18 ug | 7.59E+05 ± 0.19 |
| 33% SCF-SYN480 18 ug | 6.64E+05 ± 0.13 |
| 33% CD4-SYN480 18 ug | 6.57E+05 ± 0.79 |
| 33% CD8-SYN480 18 ug | 6.03E+05 ± 0.71 |

### EXAMPLE

### Methods:

### Plasmid Cloning:

SEQ ID NO: 24 > scFvCD133-SYN: Signal sequence of SYN is underlined - HA
epitope is in italic scFvCD133 sequence is doubled underlined - flexible
linker sequence is in bold - SYN sequence is underlined in dotted line.
GACTATGGCCCTCCCTTATCATATTTTTCTCTTTACTGTTCTTTTACCCTCTTTCACTCTCACTTATCCATATGA
TGTTCCAGATTATGCTATGGACATTGTTCTCTCCCAGTCTCCAGCAATCATGTCTGCATCTCCAGGGGAGAAGGT
CACCATATCCTGCAGTGCCAGCTCAAGTGTAAGTTATATGTACTGGTACCAGCAGAAGCCAGGATCCTCCCCCAA
ACCCTGGATTTATCGCACATCCAACCTGGCTTCTGGAGTCCCTGCTCGCTTCAGTGGCAGTGGGTCTGGGACCTC
TTACTCTCTCACAATCAGCAGCATGGAGGCTGAAGATGCTGCCACTTATTACTGCCAGCAGTATCATAGTTACCC
ACCCACGTTCGGTGCTGGGACCAAGCTGGAGCTGAAATCCTCTGGTGGCGGTGGCTCGGGCGGTGGTGGGGGTGG
TTCCTCTAGATCTTCCCTGGAAGTGAAGCTGGTGGAGTCTGGACCTGAGCTGAAGAAGCCTGGAGAGACAGTCAA
GATCTCCTGCAAGGCTTCTGGTTATACCTTCACAGACTATTCAATGCACTGGGTGAATCAGGQTCCAGGAAAGGG
TTTAAAGTGGATGGGCTGGATAAACACTGAGACTGGTGAGCCATCATATGCAGATGACTTCAAGGGACGGTTTGC
CTTCTCTTTGGAAACCTCTGCCAGCACTGCCTATTTGCAGATCAACAACCTCAAAAATGAGGACACGGCTACATA
TTTCTGTGCTACCGATTACGGGGACTACTTTGACTACTGGGGCCAAGGCACCACTCTCACAGTCTCCTCAGGCGG

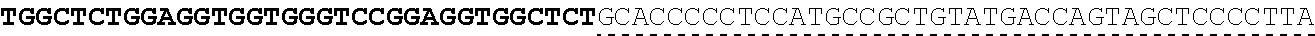

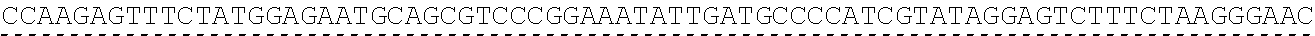

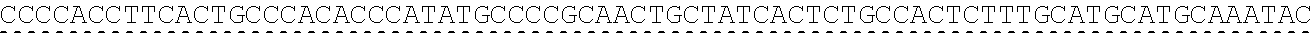

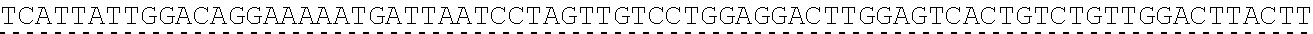

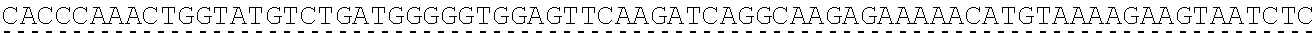

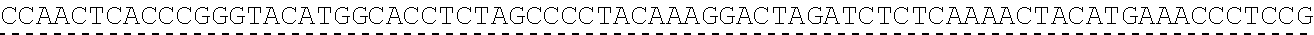

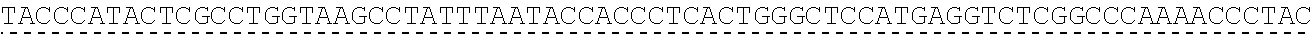

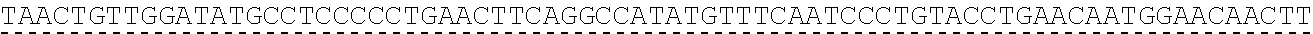

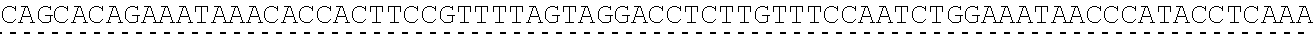

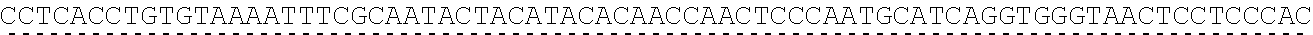

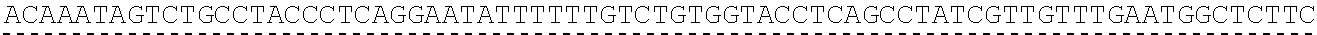

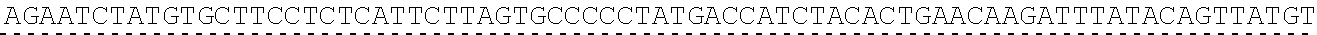

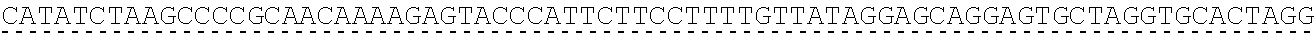

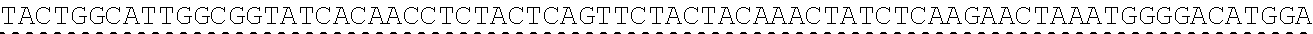

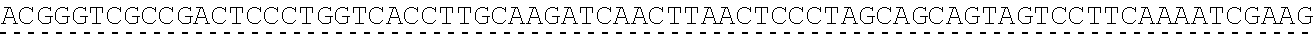

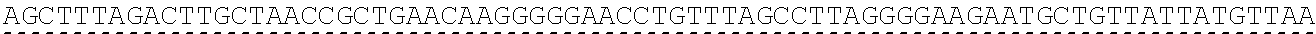

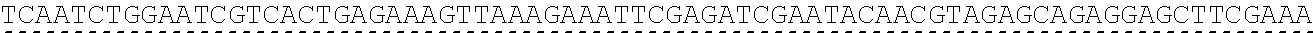

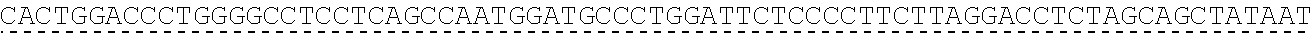

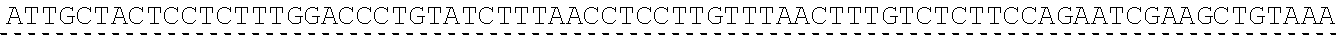

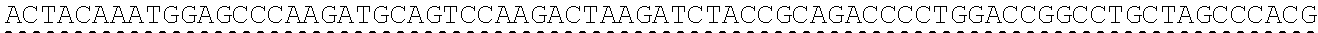

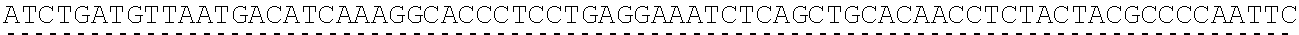

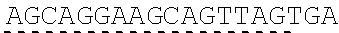

SEQ ID NO: 25 > SCF-SYN: Signal sequence of SYN is underlined - HA epitope is
in italic SCF sequence is doubled underlined - flexible linker sequence is
in bold - SYN sequence is underlined in dotted line.

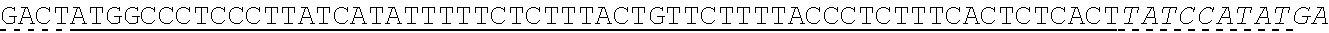

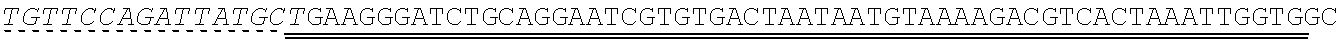

AAATCTTCCAAAAGACTACATGATAACCCTCAAATATGTCCCCGGGATGGATGTTTTGCCAAGTCATTGTTGGAT
AAGCGAGATGGTAGTACAATTGTCAGACAGCTTGACTGATCTTCTGGACAAGTTTTCAAATATTTCTGAAGGCTT
GAGTAATTATTCCATCATAGACAAACTTGTGAATATAGTGGATGACCTTGTGGAGTGCGTGAAAGAAAACTCATC
TAAGGATCTAAAAAAATCATTCAAGAGCCCAGAACCCAGGCTCTTTACTCCTGAAGAATTCTTTAGAATTTTTAA
TAGATCCATTGATGCCTTCAAGGACTTTGTAGTGGCATCTGAAACTAGTGATTGTGTGGTTTCTTCAACATTAAG

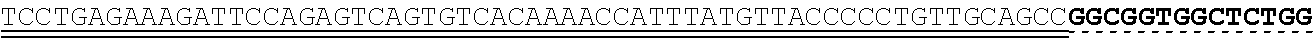

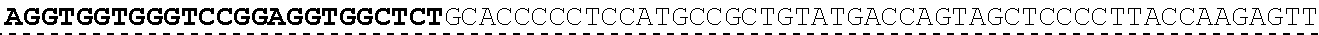

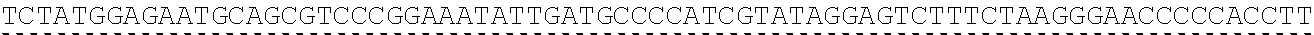

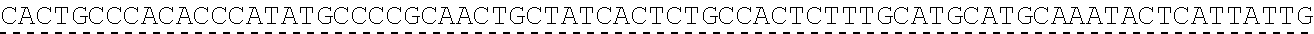

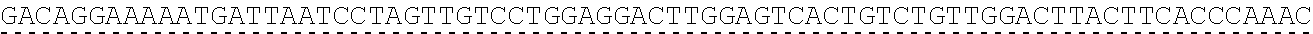

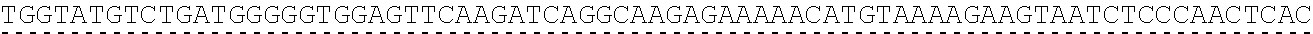

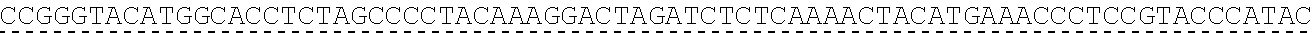

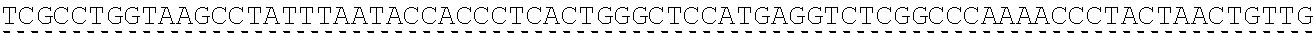

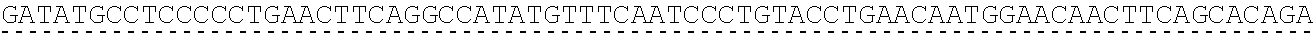

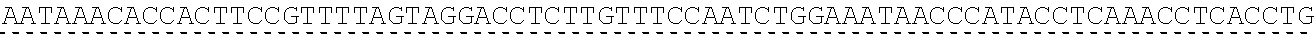

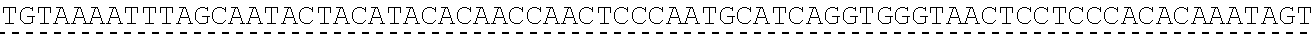

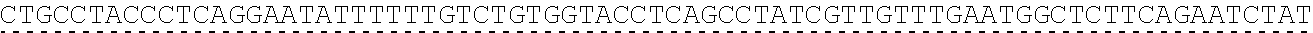

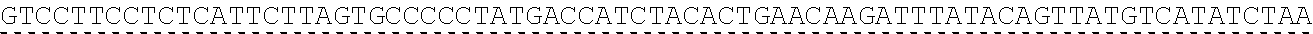

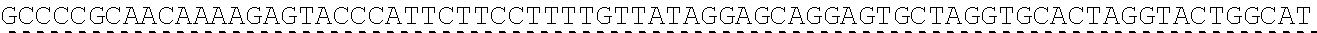

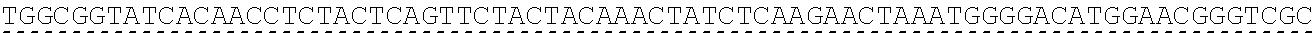

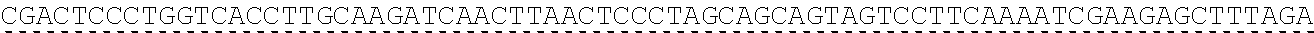

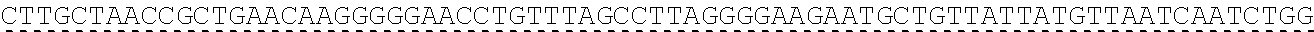

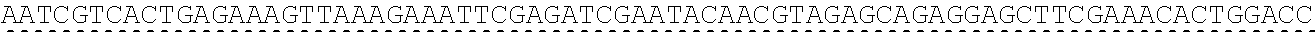

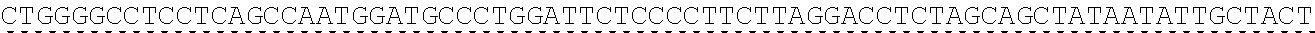

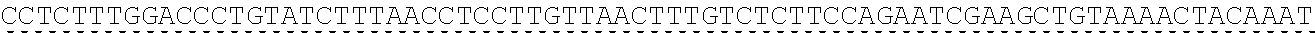

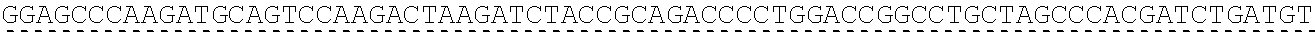

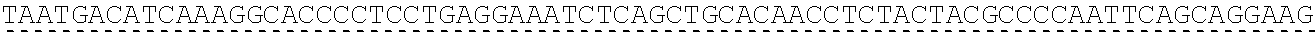

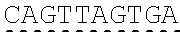

SEQ ID NO: 26 > DARPinCD4-SYN480: Signal sequence of SYN is underlined - HA
epitope is in italic SCF sequence is doubled underlined - flexible linker
sequence is in bold - SYN sequence is underlined in dotted line.

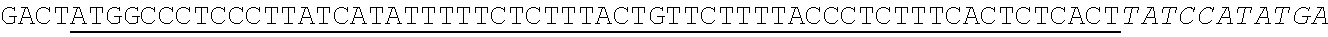

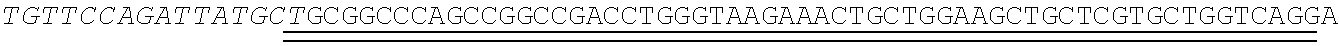

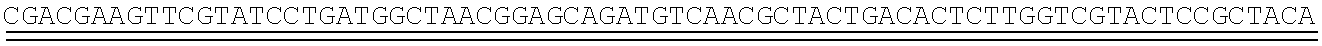

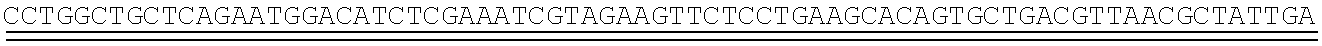

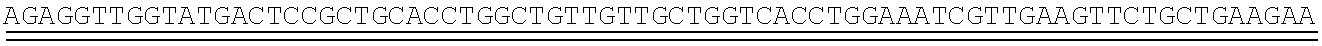

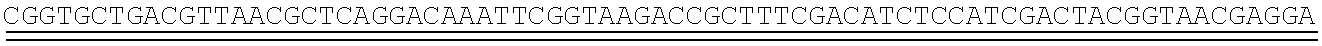

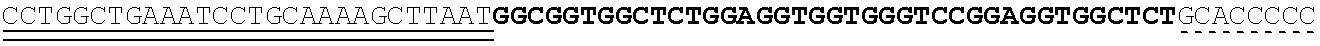

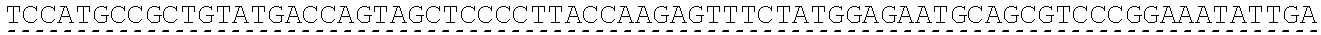

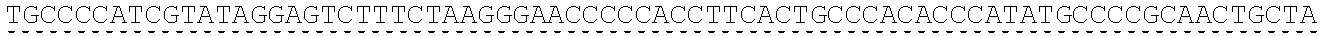

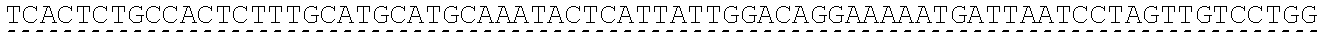

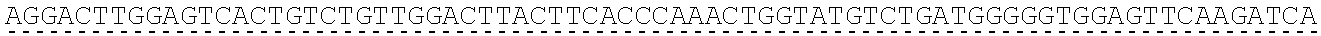

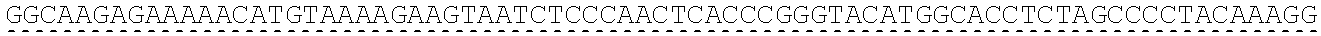

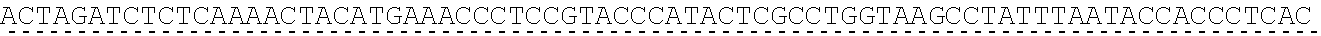

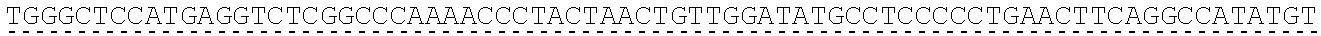

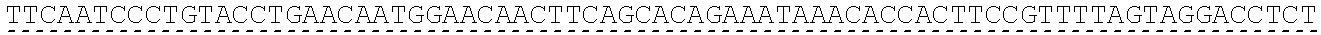

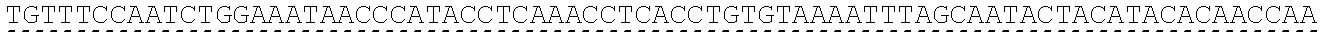

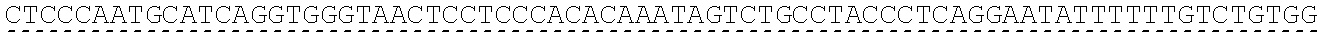

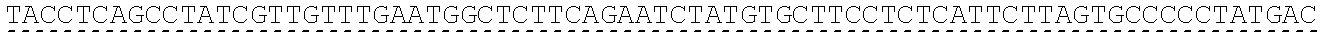

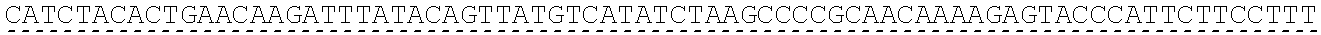

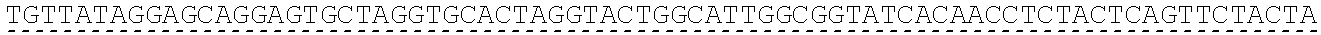

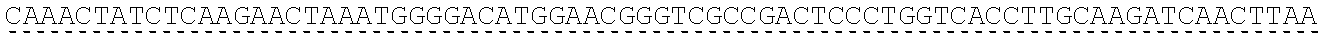

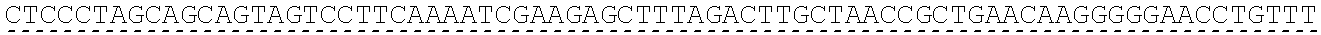

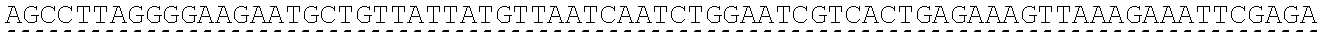

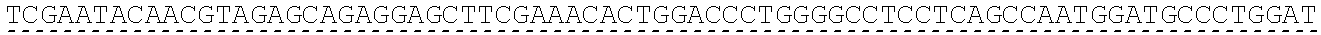

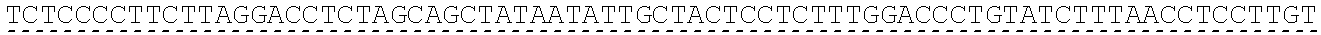

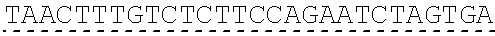

SEQ ID NO: 27 > scFvCD8-SYN480: Signal sequence of SYN is underlined - HA
epitope is in italic SCF sequence is doubled underlined - flexible linker
sequence is in bold - SYN sequence is underlined in dotted line.

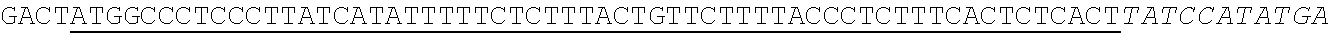

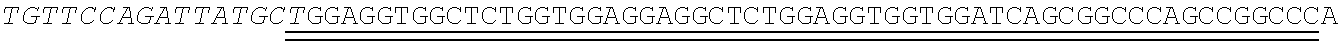

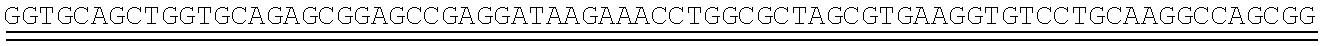

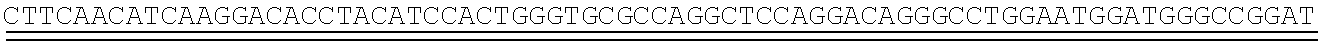

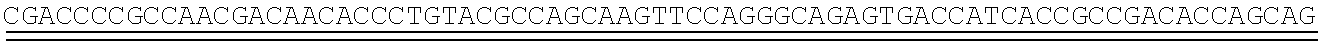

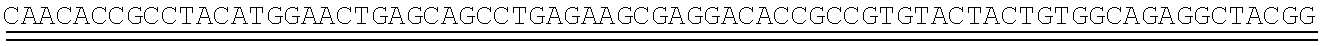

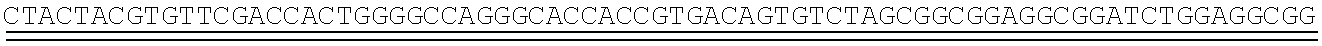

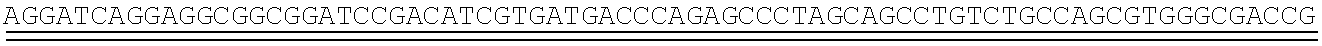

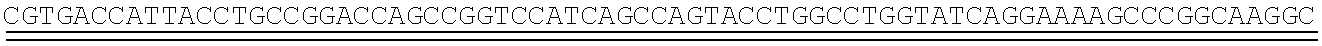

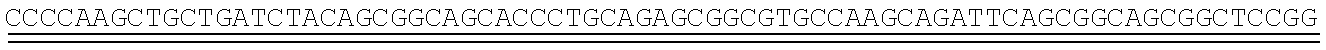

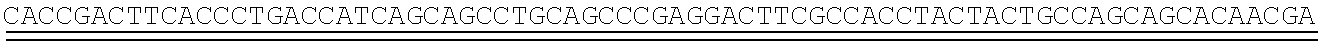

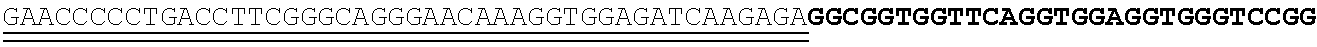

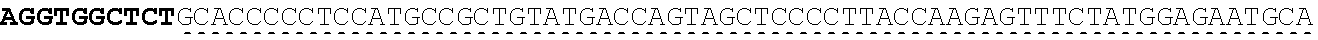

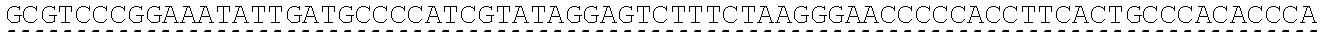

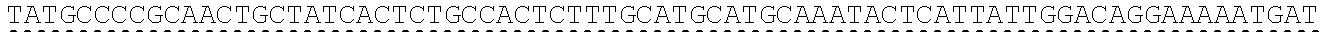

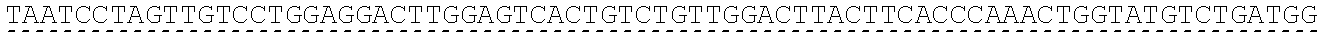

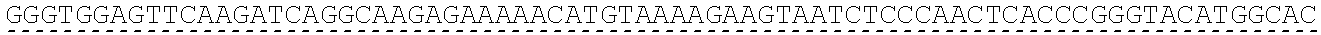

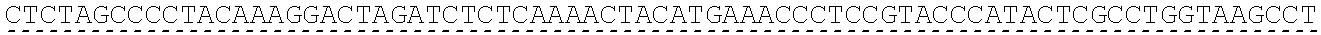

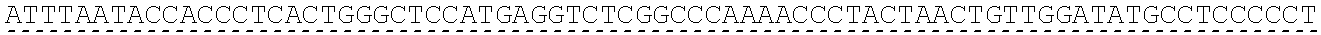

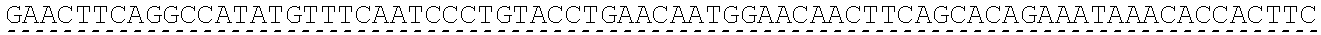

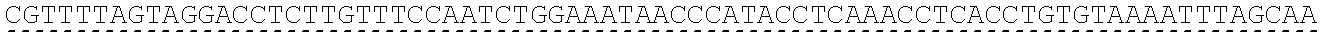

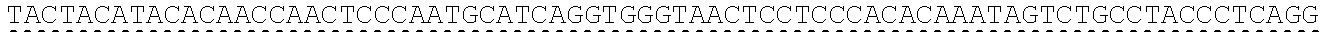

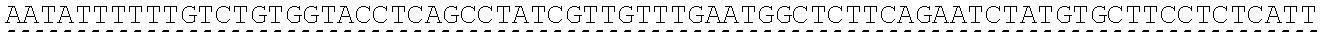

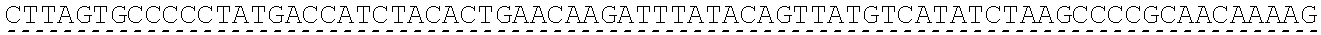

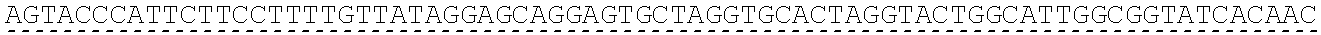

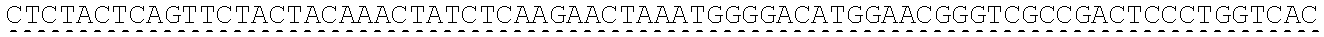

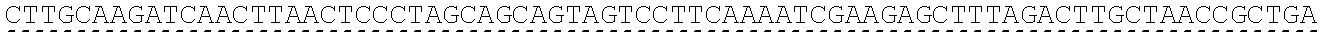

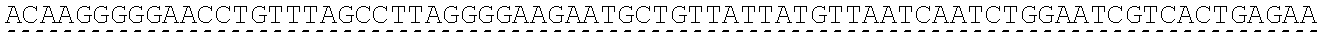

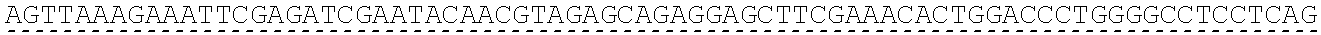

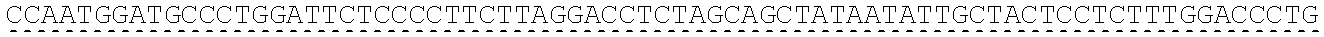

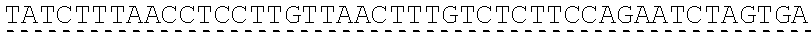

SEQ ID NO: 28 > scFVIA2-SYN480: Signal sequence of SYN is underlined - HA
epitope is in italic SCF sequence is doubled underlined - flexible linker
sequence is in bold - SYN sequence is underlined in dotted line.

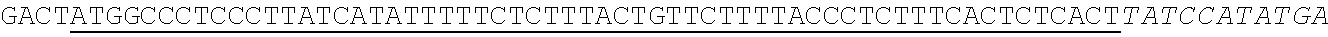

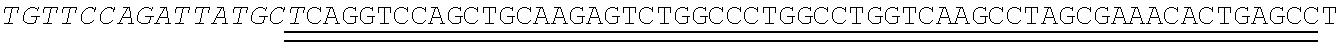

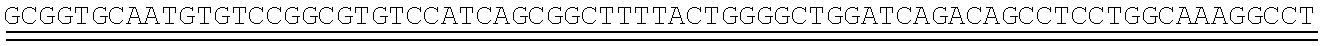

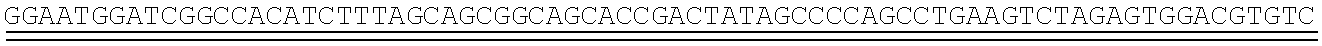

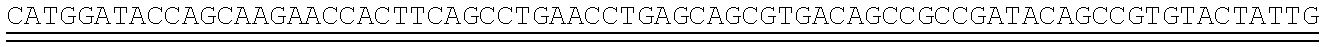

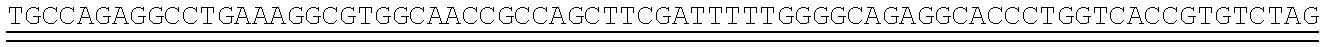

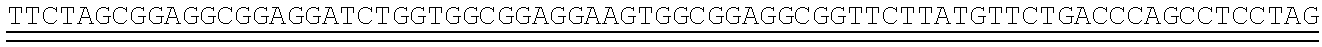

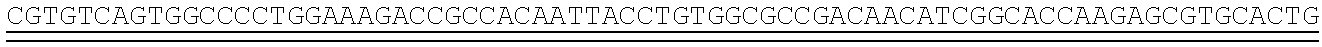

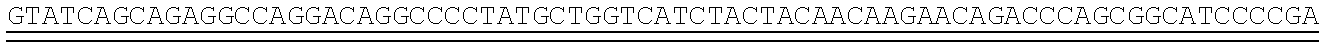

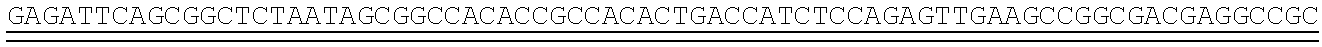

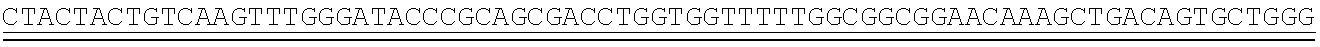

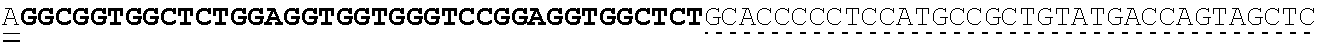

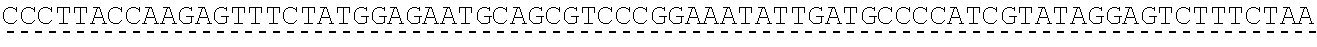

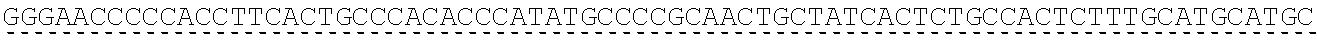

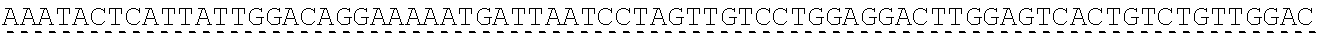

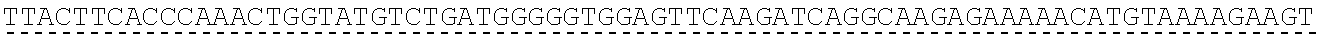

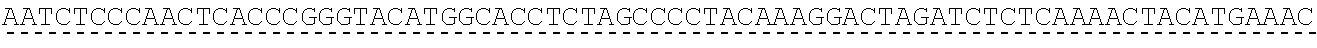

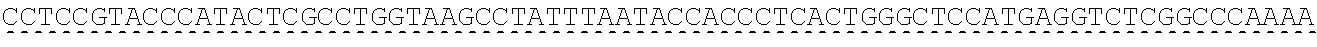

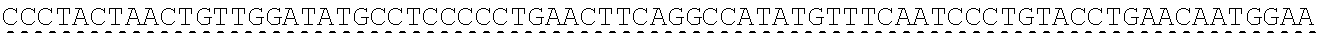

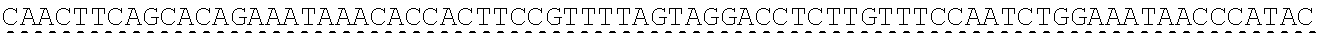

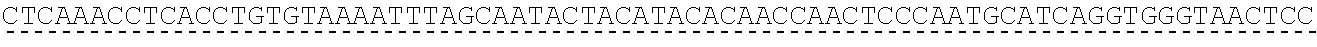

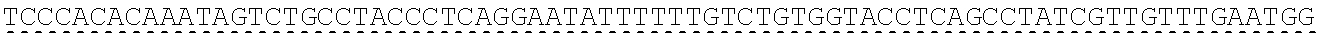

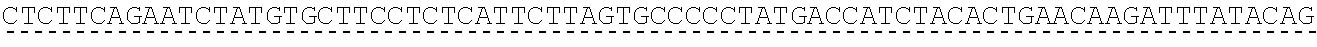

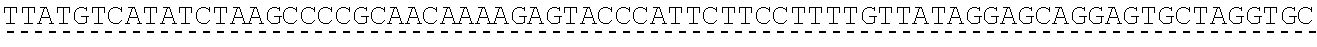

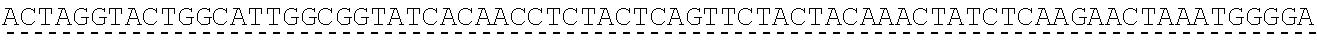

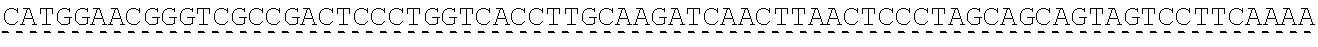

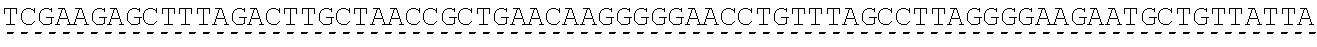

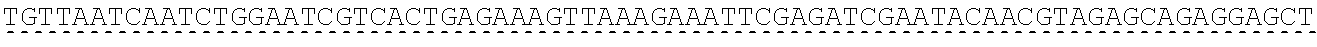

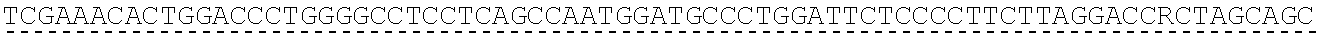

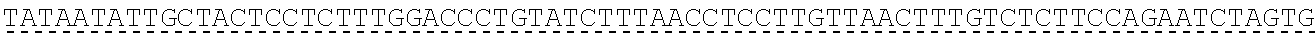

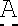

SEQ ID NO: 29 > GLP1-SYN480: Signal sequence of SYN is underlined - HA epitope
is in italic SCF sequence is doubled underlined - flexible linker sequence
is in bold - SYN sequence is underlined in dotted line.

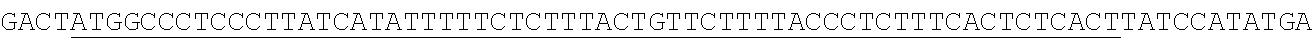

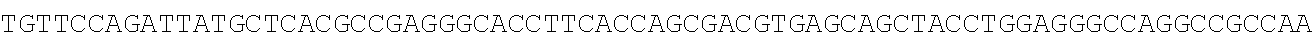

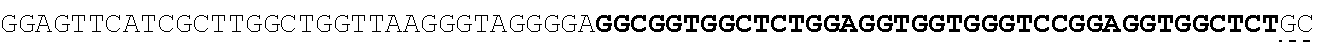

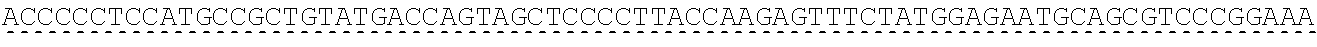

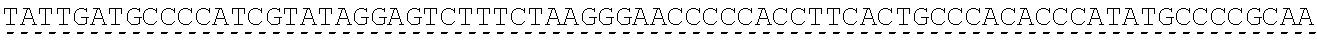

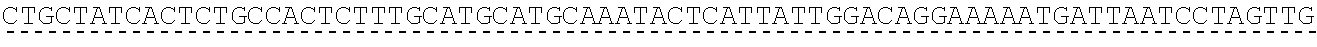

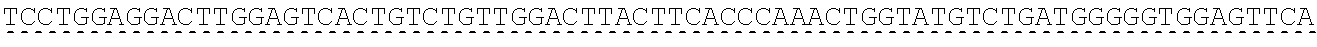

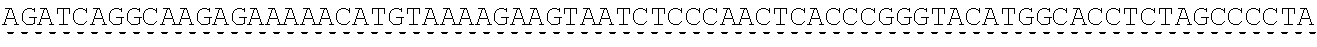

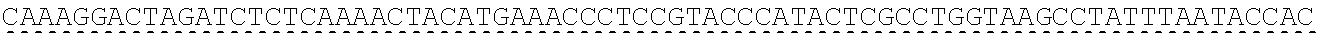

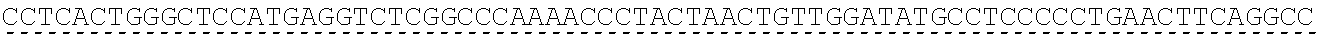

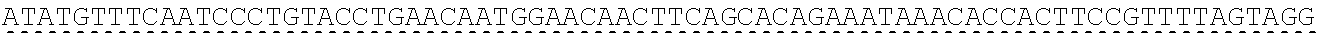

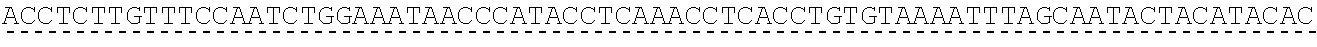

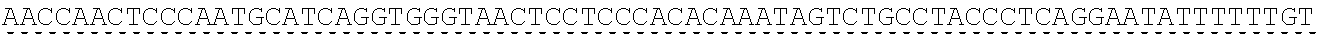

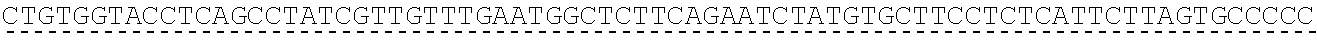

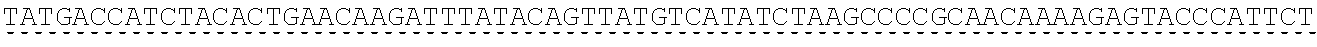

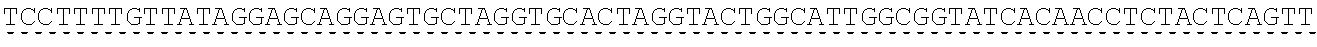

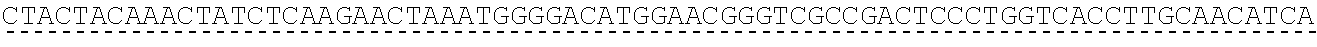

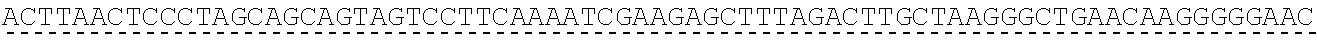

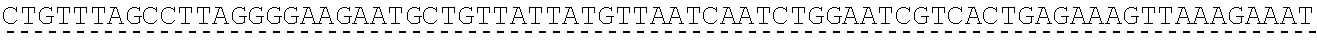

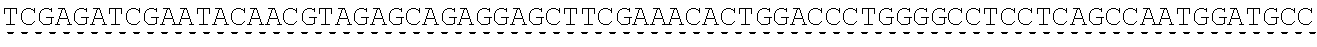

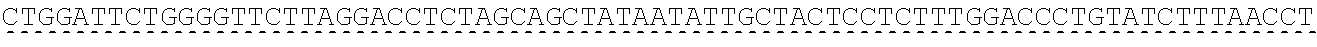

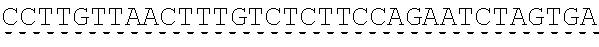

The Syncytin 1 (SYN)-coding sequence was obtained from the Ensembl database (ENST00000493463) and two point mutations determining the R393Q and F399A amino acid substitutions) were inserted to increase immunosuppressive activity of SYN². The scFvCD133-coding sequence was previously published³ and the SCF-coding sequence was obtained on gene database (Ensembl ENSG00000049130). The fragments containing scFvCD133, SYN WT and SCF were purchased by Twist Biosciences.

scFvCD133 insert was digested with EcoRI. scFvCD133-SYN insert was ligated into PMD2.G plasmid (Addgene #12259) digested with EcoRI to generate the scFvCD133-SYN plasmid. Plasmid integrity was verified by Sanger sequencing. In this plasmid, scFvCD133-SYN is under the control of a CMV promoter and enhancer.

SCF insert was digested with XbaI and BspEI. The SCF insert was ligated into the scFvCD133-SYN plasmid digested with XbaI and BspEI to generate the SCF-SYN plasmid. Plasmid integrity was verified by Sanger sequencing. In this plasmid, SCF-SYN is under the control of a CMV promoter.

SYN480 fused to SCF ligand or scFvCD133 was generated by PCR amplification of scFvCD133-SYN plasmid using the following primers:

Pf short CTD:
(SEQ ID NO: 30)
TGG GTC CGG AGG TGG CTC
Pr short CTD:
(SEQ ID NO: 31)
CCT AAC TCG AGG ACT TGA GTC ATT AGA TTC TGG
AAG AGA CAA AGT TAA C

The PCR product was digested using BspEI and XhoI and inserted into scFvCD133-SYN or SCF-SYN plasmid digested with BspEI and XhoI to generate respectively scFvCD133-SYN480 and SCF-SYN480 plasmids. Plasmid integrity was verified by Sanger sequencing. SYN480 only was generated by PCR amplification of scFvCD133-SYN480 plasmid using the following primers.

Pf:
(SEQ ID NO: 32)
TTCCTTAAGACTATGGCCCTCCCTTATCATATTTTTCTCTTTACTGTTC
TTTTACCCTCTTTCACTCTCACTGCACCCCCTCCATGCC
Pr short CTD:
(SEQ ID NO: 33)
CCT AAC TCG AGG ACT TGA GTC ATT AGA TTC TGG AAG
AGA CAA AGT TAA C

The PCR product was digested with AflII and XhoI and inserted into scFvCD133-SYN480 plasmid digested with AflII and XhoI.

Both DARPin targeting CD4 and scFv targeting CD8 sequences were obtained from patent WO2018033544. The GLP1-coding sequence was obtained from the Ensembl database (ENSG00000115263). The scFv targeting sequence of IA2 was extracted from monoclonal antibody sequence previously published⁴.

DARPinCD4, scFvCD8, scFvIA2 and GLP1 inserts were digested with AflII or XbaI and BspEI. scFvCD133-SYN480 plasmid was also digested using the same enzymes. Each insert was ligated into scFvCD133-SYN480 plasmid. Plasmid integrity was verified by Sanger sequencing. In all these plasmids, Ligand-SYN480 are under the control of a CMV promoter.

PCR products were obtained using the Phusion High-Fidelity polymerase (New England Biolabs (NEB)). Restriction enzymes were purchased from NEB.

SEQ ID NO: 34 > Glp1R-P2A-Hygro^{R}: G1P1R is underlined - P2A linker sequence
is in bold - Hygromycin sequence is double underlined.

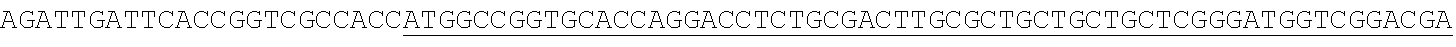

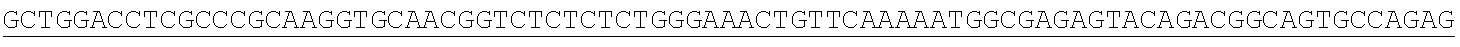

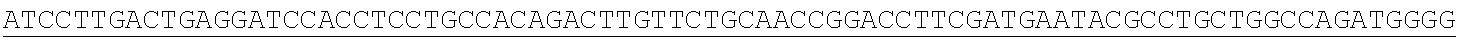

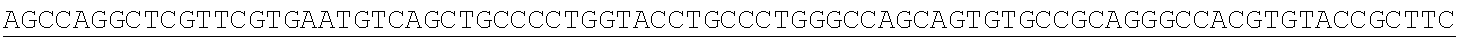

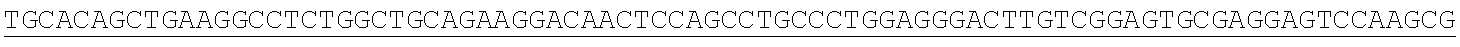

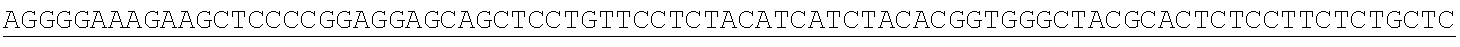

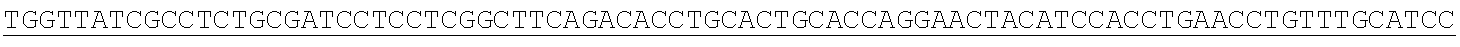

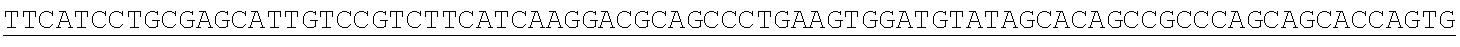

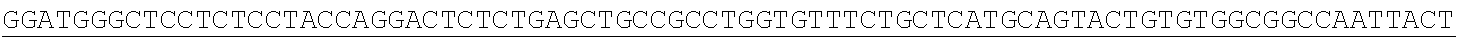

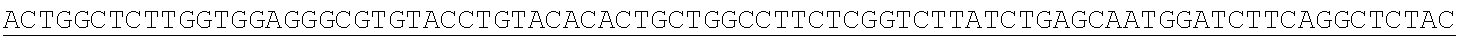

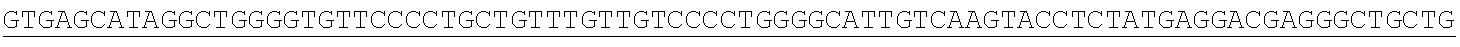

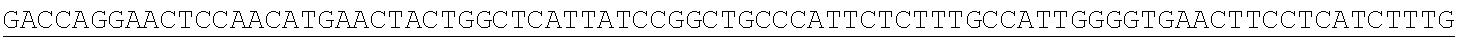

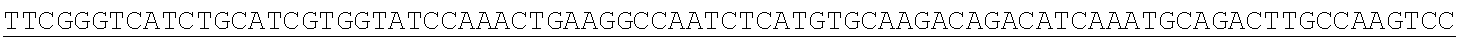

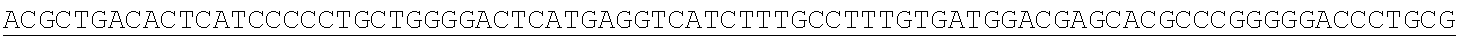

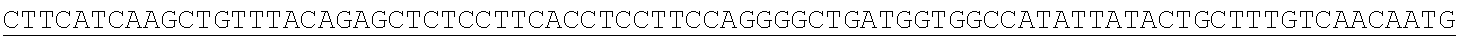

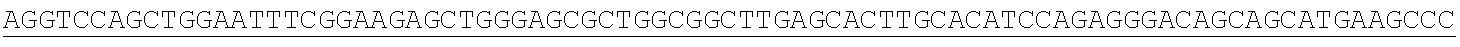

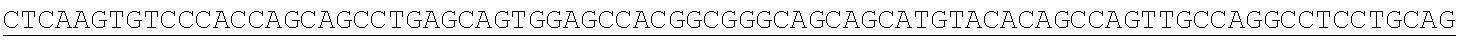

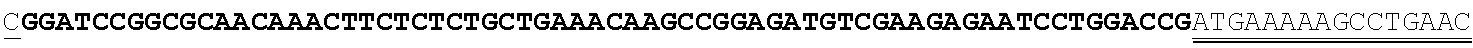

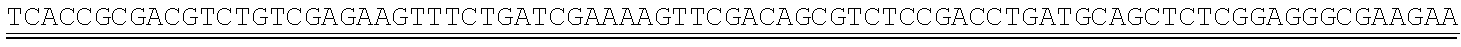

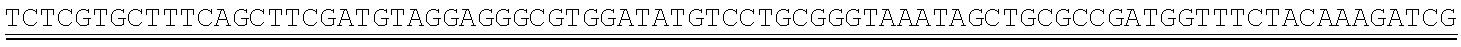

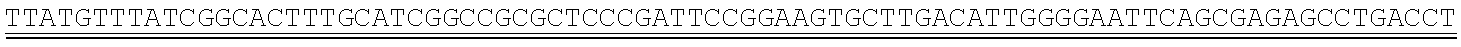

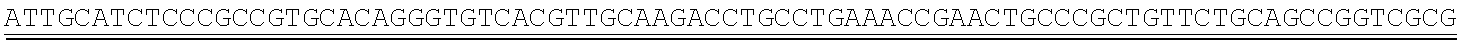

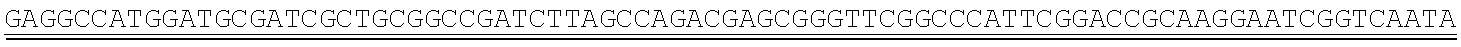

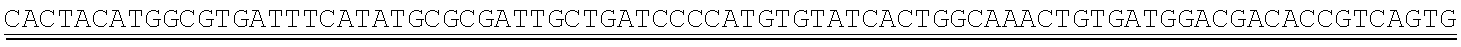

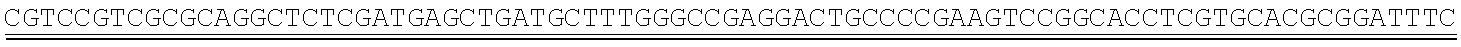

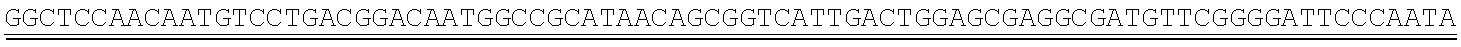

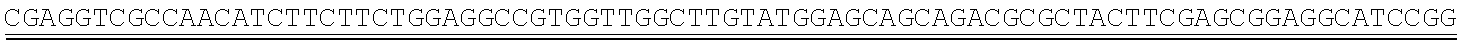

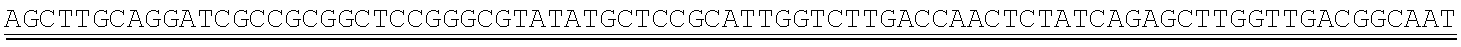

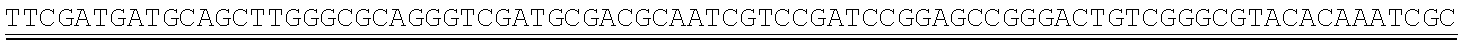

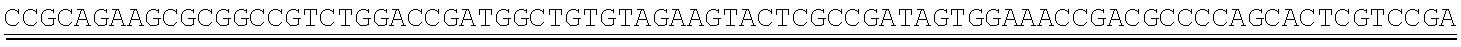

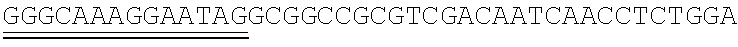

GlP1R transgene: The Glp1R-coding sequence was obtained from the Ensembl database (ENSG00000112164). The Glp1R-P2A-Hygro^{R} sequence and hygromycin insert were purchased from Twist Biosciences. PGK-GFP plasmid and Glp1R-P2A-Hygro^{R} sequence (Addgene, 19070) were digested with AgeI and SalI. Glp1R-P2A insert was ligated into the PGK-GFP plasmid. Plasmid integrity was verified by Sanger sequencing.

PCR products were obtained using the Phusion High-Fidelity polymerase (NEB). Restriction enzymes were purchased from NEB.

### LV Production

HEK 293T cells were cultured in DMEM+Glutamax supplemented with Glutamax (Gibco), Non-Essential Amino Acid (Gibco) and Pen/Strep (Gibco). The medium was changed 2 h before transfection. 293T cells were transfected when they reach 80 to 90% of confluency with the following plasmids: (i) Envelope-expressing plasmid (0.7 μg for P60 plates (21 cm²) and 6 μg or 12 μg or 18 μg for P150 plates (152 cm²)), (ii) pRSV-Rev plasmid (Addgene, 12253) (1.1 μg for P60 plates (21 cm²) and 7.25 μg for P150 plate (152 cm²)); (iii) pMDlg/pRRE plasmid (Addgene, 12251) (2.2 μg for P60 plates (21 cm²) and 14.5 μg for P150 plate (152 cm²)); and (iv) PGK-GFP transfer plasmid (Addgene, 19070) (3 μg for P60 plates (21 cm²) and 18 μg for P150 plate (152 cm²)). We used PEI as a transfection reagent at a 1:3 DNA:PEI ratio. The transfection mix was prepared in DMEM and added dropwise on HEK 293T cells. Media was changed between 12 to 16 h after transfection. Viral supernatant was collected 24 h later, centrifuged 5 min at 500 g, filtered using 0.45 μm filters and concentrated by ultracentrifugation for 2 h at 100,000 g at 4° C. Viral pellet was resuspended in the media used for viral transduction (StemSpan or X-VIVO 20 or PBS) and either used directly or stored at −80° C.

### LV Titration:

Physical particle titers of VSV-G- or SYN-pseudotyped LVs were determined by p24 ELISA (Alliance© HIV-1 Elisa kit, Perkin-Elmer, Villebon/Yvette, France).

### Cell Culture and Transduction:

We obtained human CB CD34⁺ HSPCs from healthy donors. CB samples eligible for research purposes were obtained because of a convention with the CB bank of Saint Louis Hospital (Paris, France).

HSPCs were purified by Ficoll gradient centrifugation (Eurobio, les Ulis, France) and by CD34⁺ magnetic beads sorting (Miltenyi Biotec, Bergisch Gladbach, Germany). Cells were stored in liquid nitrogen. HSPCs were thawed from 48 h to 96 h before transduction and cultured in X-VIVO or StemSpan (STEMCELL Technologies) medium supplemented with the following cytokines (Pepro Tech): stem cell factor (SCF) (300 ng/ml), Flt-3L (300 ng/ml), thrombopoietin (TPO) (100 ng/ml), interleukin-3 (IL-3) (60 ng/ml) and StemReginin1 (250 nM) (STEMCELL Technologies).

If required, HSPCs were stained with anti-CD117 or anti-CD133 (Miltenyi Biotech) antibodies and FACS-sorted using SH800 Cell Sorter (Sony Biotechnology). Cells (10⁶ cells/mL) were transduced overnight with LVs in the presence of VF1 (12 μg/mL) (Miltenyi Biotech), then washed with PBS and resuspended in fresh X-VIVO 20 supplemented with cytokines mentioned above. The following day, HSPCs were analyzed for GFP expression by flow cytometry on a Fortessa X20 (BD Biosciences) analyzer using Diva and FlowJo v10 (BD-Biosciences) softwares.

200,000 to 250,000 HEK 293T cells were transduced overnight with LV in the presence of VF1 (12 μg/mL). The following day, the medium was removed and replaced by fresh medium. The third day, 293T HEK cells were analyzed for GFP expression by flow cytometry on a Fortessa X20 (BD Biosciences) analyzer using Diva and FlowJo v10 (BD-Biosciences) softwares.

CD4⁺ and CD8⁺ T cells were purified by Ficoll gradient centrifugation (Eurobio, les Ulis, France) and by CD4 or CD8 magnetic beads sorting (Miltenyi Biotec, Bergisch Gladbach, Germany). Cells were activated during 3 days with PHA (2.5 g/mL, MilliporeSigma) in Panserin 401 (Pan Biotech) supplemented with 5% human AB serum (Bio West), penicillin (100 UT/mL), and streptomycin (100 μg/mL). After 3 days, dead cells were removed by Ficoll gradient centrifugation, and cells were cultured in RPMI1640+10% FBS+IL-2 (100 UI/mL). 100,000 Cells (10⁶ cells/mL) were transduced overnight with LVs in the presence of VF1 (12 μg/mL) (Miltenyi Biotech), then washed with PBS and resuspended in fresh medium supplemented with IL-2 as mentioned above. The seventh day, CD4⁺ and CD8⁺ T cells were analyzed for GFP expression by flow cytometry on a Fortessa X20 (BD Biosciences) analyzer using Diva and FlowJo v10 (BD-Biosciences) softwares.

### Flow Cytometry Staining

300.000 HEK 293T cells or HCT 116 were stained with either anti-IA2 (ThermoFischer) or anti-Glp1R antibodies (ThermoFischer). Stained cells were incubated with secondary antibodies (anti-Rabbit IgG) coupled with Alexa Fluor 647. 200,000 CD4⁺ and CD8⁺ T cells were stained with either anti-CD4 (BioLegend) or anti-CD8 (BioLegend) antibodies. Cells were analyzed for respective marker expression by flow cytometry on a Fortessa X20 (BD Biosciences) analyzer using Diva and FlowJo v10 (BD-Biosciences) softwares.

### Overexpressing Cell Lines

LV were produced as mentioned above but transfer plasmid PGK-Glp1R-P2A-BleoR was used. Viral supernatant was collected, centrifuged and filtered as mentioned above. 5.105 I HEK 293T cells or HCT116 cells were transduced with fresh viral supernatant collected after 24 h. 48 h after transduction, hygromycin was added (300 g/mL) for selection for 14 days. GlP1R expression was confirmed by flow cytometry analysis.

### Vector Copy Number

Genomic DNA (gDNA) was extracted using PureLink Genomic DNA kit according to manufacturer's instructions (Invitrogen). For CB HSPCs, digital droplet dPCR (ddPCR) was performed 13 days post transduction. VCN was analyzed according to the protocol described by Corre et al.⁸.

For HEK 293T cells and HCT 116 cells, ddPCR was performed 7 days after transduction. Amplification of the human ALB gene with Alb For, Alb Rev and Alb Pro was used to determine the number of diploid genomes. GFP For, GFP Rev and GFP PRO were used to determine the vector copies.

Primers and probe sequences are reported below:

```
(SEQ ID NO: 35)
Alb For: 5′-GCTGTCATCTCTTGTGGGCTGT-3′
  (SEQ ID NO: 36)
Alb Rev: 5′-ACTCATGGGAGCTGCTGGTTC-3′
  (SEQ ID NO: 37)
Alb Pro: 5′-CCTGTCATGCCCACACAAATCTCTCC-3′ [VIC]
  (SEQ ID NO: 38)
GFP For: 5′-ATG GTG AGC AAG GGC GAG GA-3′
(SEQ ID NO: 39)
GFP Rev: 5′-CTT CAG GGT CAG CTT GCC GTA-3′
  (SEQ ID NO: 40)
GFP Pro: [FAM] 5′-AAA CGG CCA CAA GTT CAG
CGT GTC C-3′ [MGBEQ]
```

The reaction was performed on the Biorad system (Biorad QX200 autoDG) according to the manufacturer recommendations using 10 units of the DraI restriction enzyme in the mix (Biorad ddPCR Supermix for Probes (No dUTP)) and 30 ng of gDNA was used for each reaction.

### Results:

We developed a new system to modify tropism of syncytins, envelope proteins, which can be used to pseudotype viruses or viral-like particles (VLPs), and used for gene transfer or other applications. Syncytins are encoded by genes from endogenous retroviruses, which have entered the germline of mammalian hosts⁵. The structure of syncytins resembles that of a typical retroviral envelope glycoprotein. In addition, syncytins have immunosuppressive properties, which make them relevant for potential in vivo well-tolerated gene delivery.

### LVs Specifically Human HSPCs

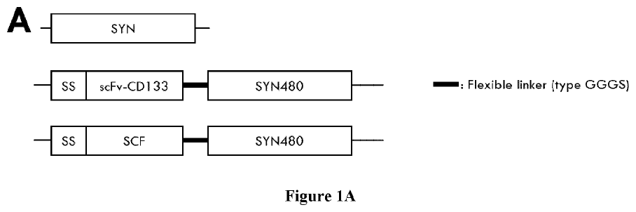

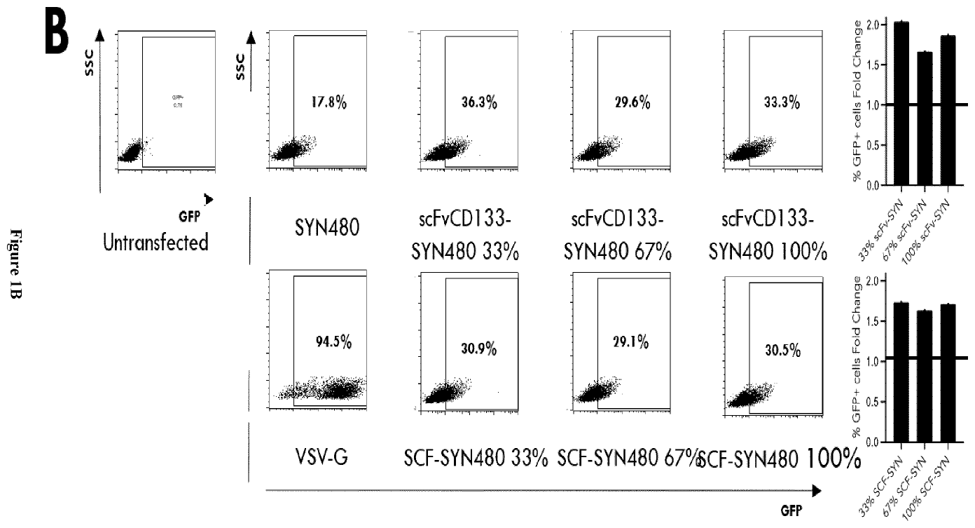

We created a fusion protein containing the Syncytin-1 (SYN) signal sequence (SS), a ligand (either natural or engineered), the SYN protein and a flexible linker between SYN and the ligand (FIG. 1A) to enhance transduction of the cell type expressing the receptor or the antigen targeted by the ligand, for instance hematopoietic stem progenitor cells (HSPCs). We use as ligands either Stem Cell Factor (SCF), which binds CD 117 (c-kit) receptor or a single-chain fragment variant (scFv) directed against CD133 receptor (scFvCD133). Both ligands are known markers of HSPCs^{6,3}. In order to evaluate the efficiency of our strategy and to determine the best envelope configuration, we transduced Cord Blood (CB) HSPCs with lentiviral particles containing a GFP-encoding mRNA and pseudotyped with different ratios of our fusion protein to wild-type SYN. To this aim, we produced lentiviral vectors (LVs) by transfecting HEK 293T cells with different stoichiometric ratios of the plasmid encoding for wild type (WT) SYN to either the SCF-SYN- or the scFvCD133-SYN-expressing plasmid. The lentiviral particles produced using 33%, 67% or 100% ratios were selected based on the natural conformation of SYN, which is a trimeric protein. Thus, we hypothesized that, using these ratios, each envelope protein would be composed of one (33% ratio), two (67% ratio), or three (100%) ligand-SYN monomer, the remaining monomers being WT SYN.

We observed a low transduction efficiency in HSPCs using WT SYN (17%) compared to VSV-G pseudotyped LV, but a substantial increase when SYN was fused to a ligand. Independently of the ratio, fusion of SCF or scFvCD133 with SYN increased the percentage of GFP⁺ CB HSPCs by almost two-fold (FIG. 1B), indicating that addition of the ligand can increase LV transduction if cells harbor the targeted receptor or antigen on their surface. Interestingly, lentiviral particles harboring only the ligand-SYN envelope (100%) were able to efficiently transduce HSPCs. Therefore, to potentially increase the specificity of these LVs, we used LVs produced using a 100% ratio for the following experiments.

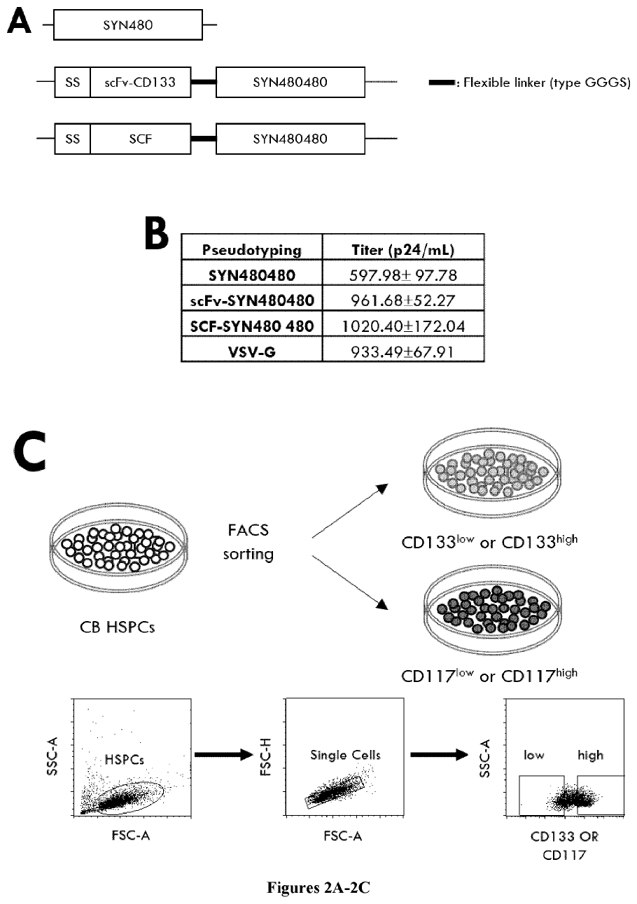

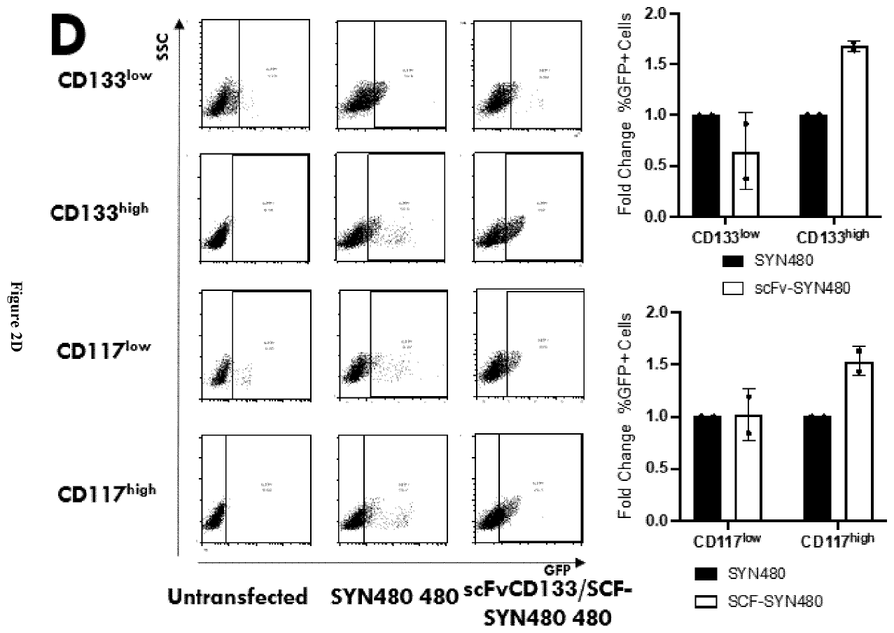

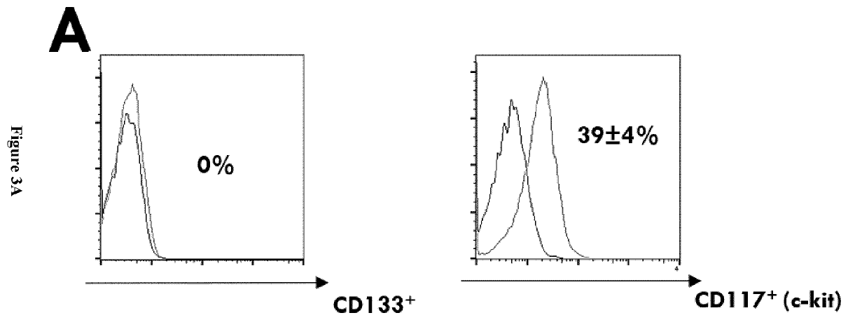

It has been previously reported that deletion of the last amino acids of SYN's cytoplasmic tail increased its fusion capacity^{7,8}. Therefore, we designed new fusion proteins by replacing SYN with its C-terminally truncated form, containing 480 instead of 538 amino acids (SYN480; FIG. 2A). LVs pseudotyped with these different engineered envelope proteins (scFvCD133- and SCF-SYN480) showed titers similar to VSV-G pseudotyped LVs and roughly two-fold higher compared to SYN480 pseudotyped LVs, as measured by p24 ELISA (FIG. 2B). Noteworthy, batches of LVs pseudotyped with SYN480 could not be produced by pooling two consecutive harvests from the same producing cells collected 48 and 72 h post-transfection, due to fusion of HEK 293T cells transfected with SYN480-expressing plasmids (data not shown), as previously reported with WT SYN⁹. On the contrary, HEK 293T cells transfected SCF-SYN480- and scFvCD133-SYN480-expressing plasmids do not fuse or only modestly (data not shown), allowing consecutive lentiviral harvests from the same producing HEK 293T cells. Thus, adding a ligand to SYN facilitates LV production, by increasing viral titers and by allowing multiple harvests from the same producing cells.

To evaluate the preferential targeting of cells expressing CD117 or CD133 by SCF-SYN480 and scFvCD133-SYN480, respectively, we compared transductions levels in CB HPSC subpopulations expressing different CD117 or CD133 levels. CB HSPCs were sorted according to either CD133 or CD117 expression levels and transduced with the different LVs (FIG. 2C). Transduction of the CD133^{high} population with scFvCD133-SYN480 LV resulted in a 69% increase of GFP⁺ cells (FIG. 2D). Similar results were observed in CD117^{low} and CD117^{high} cell populations, with SCF-SYN480 LV showing an increased transduction efficiency in CD117^{high} CB HSPCs (55% increase). Therefore, addition of a ligand to SYN480 resulted in an increased specificity towards the desired cell type expressing the targeted antigen.

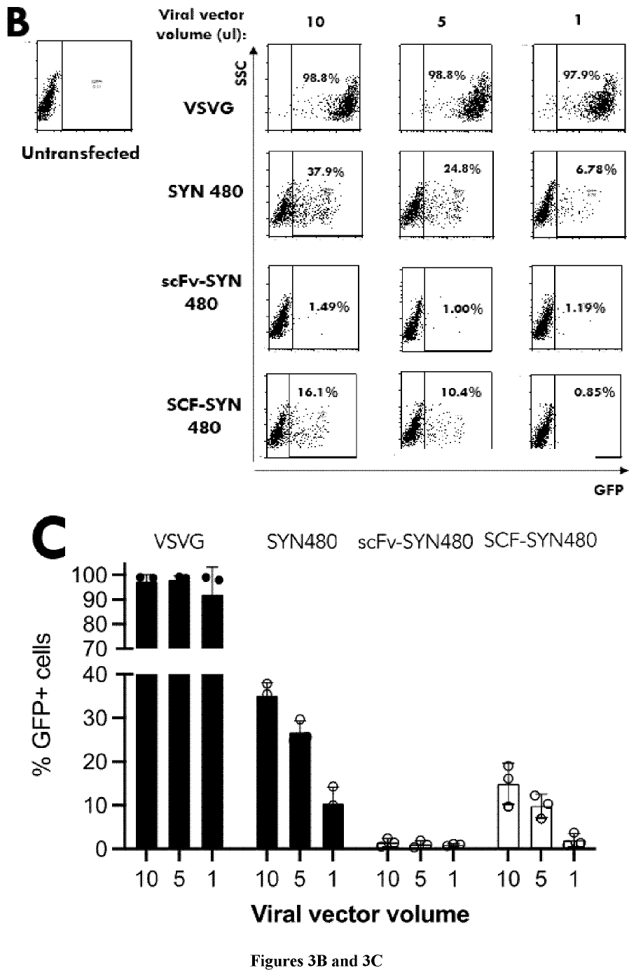

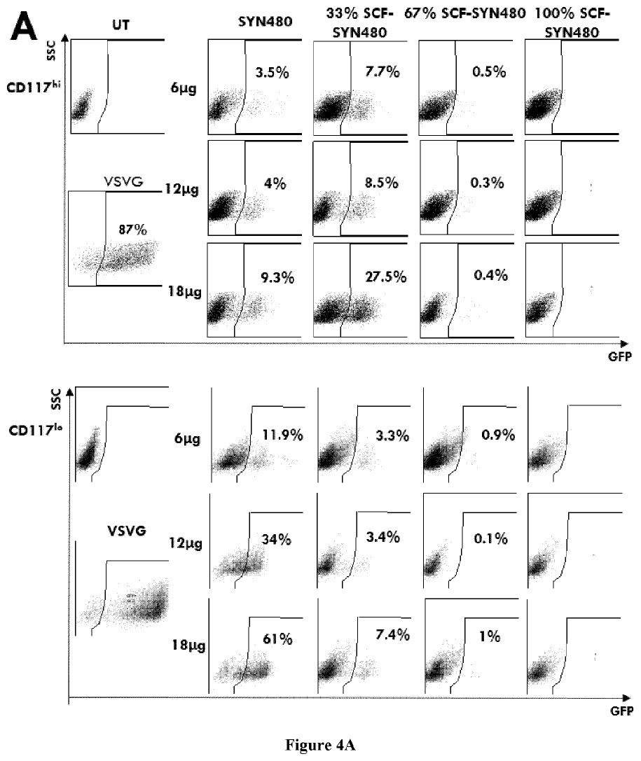

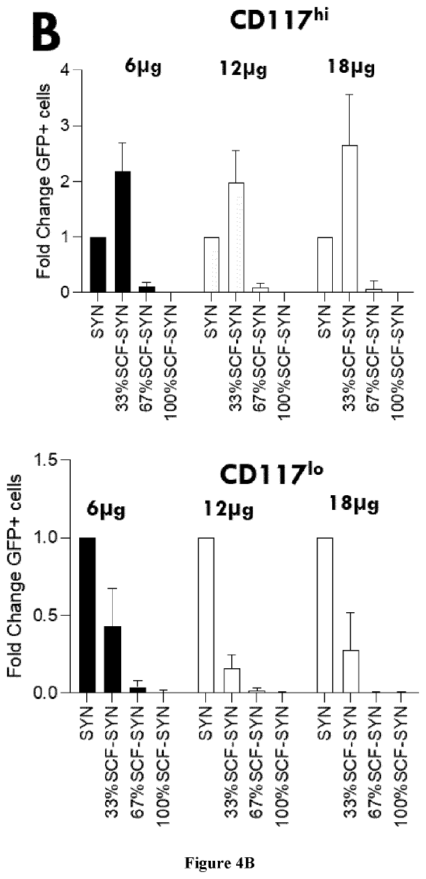

Finally, we asked whether the fusion of SYN480 to a ligand affects SYN480 binding to a cell type not expressing the receptor targeted by the ligand. Therefore, we assessed the transduction efficiency of scFvCD133-SYN480 or SCF-SYN480 LV in HEK 293T cells, which do not express CD133 and express low CD117 levels (39% positive cells) (FIG. 3A). SYN480 LVs showed a transduction efficiency of 35%, which is higher compared to that previously reported for WT SYN (˜25%)⁹. Fusion of scFvCD133 to SYN480 almost abolished binding of SYN480 to HEK 293T cells, with transduction frequency dropping from 35% to <1% (FIGS. 3B and 3C), indicating a full retargeting of SYN480's tropism. Moreover, SCF-SYN480 lentiviral particles showed a decreased transduction efficiency compare to SYN480 LV (from 35% to 15%), indicating that SCF-SYN480 might bind only to HEK 293T cells expressing CD 117 on their surface (FIGS. 3B and 3C) and suggesting a partial retargeting of SYN480's tropism.

As we observed GFP⁺ transduced HSPCs but with low fluorescence intensity (FIGS. 1 and 2), we asked whether we could improve GFP intensity signal in transduced cells. It has been shown with several viral envelope proteins that the amount of plasmid used to produced viral particles affect the titer and infectivity¹⁰. Therefore, we decided to transfect HEK 293T cells with different amounts of envelope plasmids to produce our LVs (6 μg, 12 μg and 18 g). The lentiviral particles produced using 33%, 67% or 100% ratios were selected based on the natural conformation of SYN, which is a trimeric protein, as mentioned earlier.

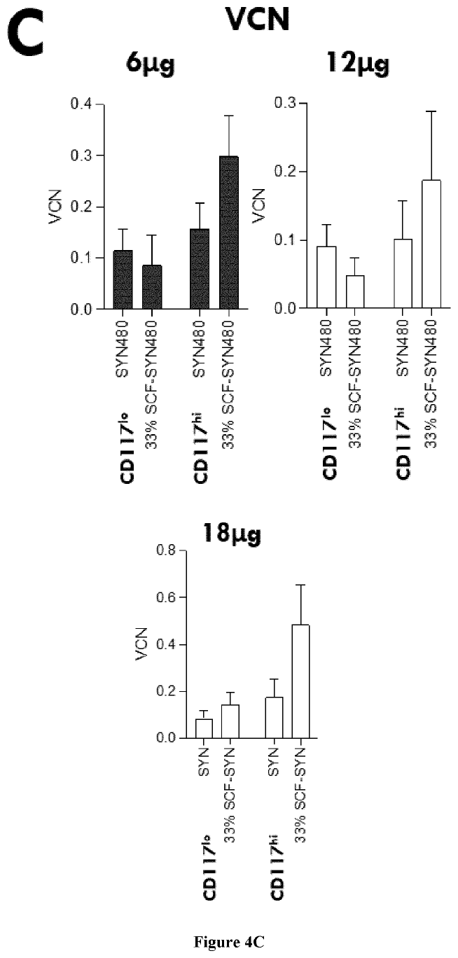

Following the same strategy as before, we tested our new LVs in CB HPSCs subpopulations expressing different CD117 levels. We also focused our analysis on GFP⁺ cells with higher fluorescence intensity. Using SYN480, we observed a low transduction efficiency in CD117^{high} HSPCs that increased in a dose-dependent manner, ranging from 3±0.8% to 8±2% with 6 g and 18 μg as amount of plasmid used to produce the viral particles, respectively (FIGS. 4A and 4B). LVs produced using the highest amount of plasmid seems to have better ability to transduce HSPCs, despite a lower titer compared to the LV produced using the lowest amount of plasmid (Table 1). By fusing SYN480 to a ligand, we observed a substantial increase in transduction efficiency. Using 33% of SCF-SYN480 envelope, we were able to increase the percentage of GFP⁺ CD117^{high} HSPCs by at least two-fold, independently of the amount of plasmid used to pseudotype the LV (FIGS. 4A and 4B). The transduction efficiency increased with the increase of the amount of SCF-SYN480 envelope plasmid used, as observed with SYN480 LVs. While 33% of SCF-SYN480 envelope was able to increase transduction efficiency, we observed very low levels of GFP⁺ cells using 67% of SCF-SYN480 and no GFP⁺ cells using 100% of SCF-SYN480, suggesting than having two or three SYN480 chain fused to a ligand affects the infectivity of the LV.

We observed a similar trend in CD117^{low} HSPCs transduced with SYN480 only, with an increase of transduction levels with the highest amount of plasmid used to produce the LVs. On the contrary, the percentage of GFP⁺ CD117^{low} w cells was strongly decrease using 33% of SCF-SYN480 envelope independently of the amount of envelope plasmid used, partially preventing SYN480 envelope to binds to its natural receptor. Regarding the 67% and 100% ratios between SCF-SYN480 and SYN480, we obtained extremely low levels of transduction efficiency or no transduction efficiency, respectively (FIGS. 4A and 4B).

Finally, we assessed transduction efficiency and proviral integration in the genome by analyzing vector copy number (VCN) in both CD117^{high} and CD117^{low} HSPCs 13 days after transduction with LVs pseudotyped either with SYN480 or with 33% of SCF-SYN480. We used a protocol to specifically quantify proviral integration events and not episomal, non-integrated proviruses (i.e., pseudotransduction)¹¹. We observed consistent increase of VCN in CD117^{high} using 33% SCF-SYN480 compared to SYN480, independently of the amount of envelope plasmid used, which is consistent with our flow cytometry analysis (FIGS. 4A, 4B and 4C). Increased fold changes in VCN levels were consistent with those observed for GFP⁺ cells, confirming increased transduction efficiency and retargeting of natural SYN480's tropism by addition of a ligand targeting a receptor present on the target cells' surface. Lastly, we observed higher VCN using the highest envelope plasmid amount (FIG. 4C). VCN observed in CD117^{low} HSPCs were decreased using 6 and 12 μg of SCF-SYN480 envelope compared to SYN480 envelope, confirming flow cytometry data and the retargeting of SYN's tropism towards CD117^{high} HSPCs. Noteworthy, decrease of VCN was not as strong as the decrease of the percentage of GFP⁺ cells.

### LVs Specifically Human T Cells

To demonstrate that our approach could be applied to target other cell types using ligands targeting their specific cell surface receptors, we decided to focus on immune T cells, notably CD4⁺ and CD8⁺ cells.

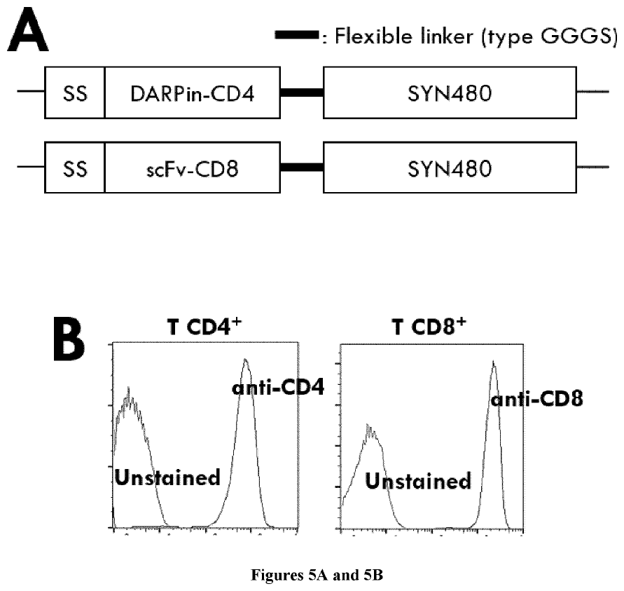

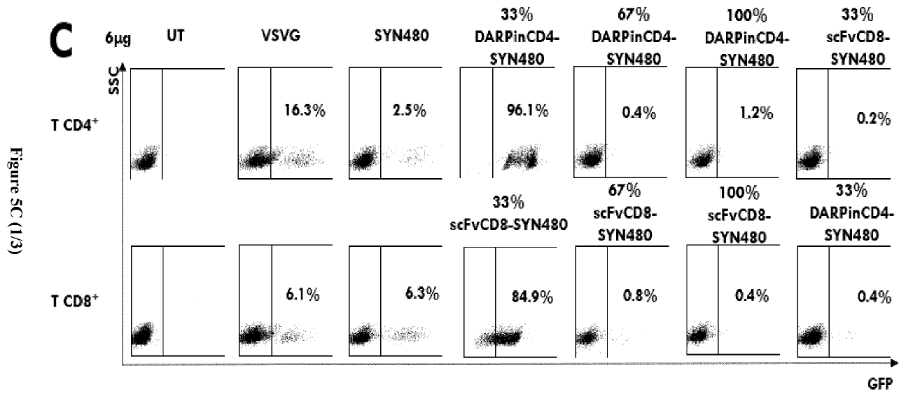

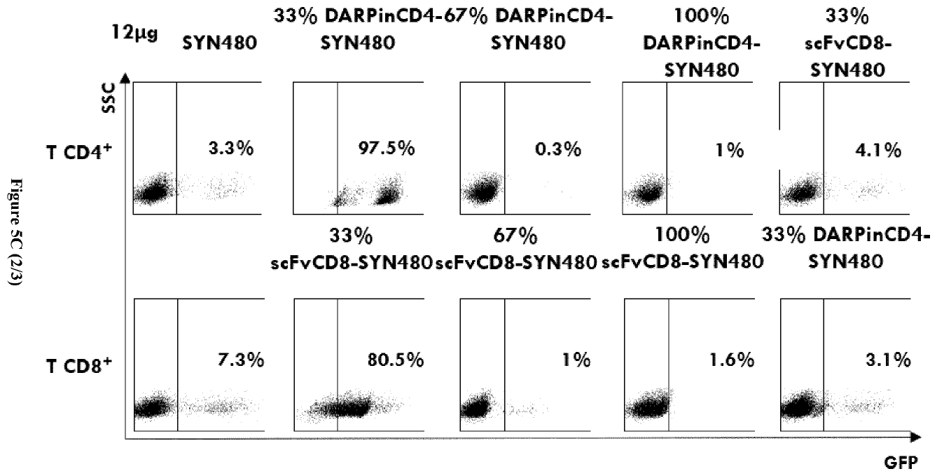

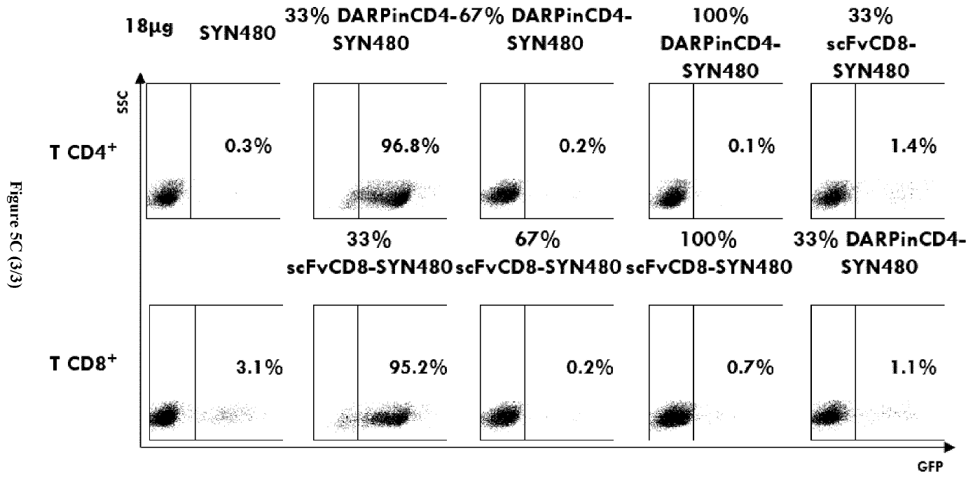

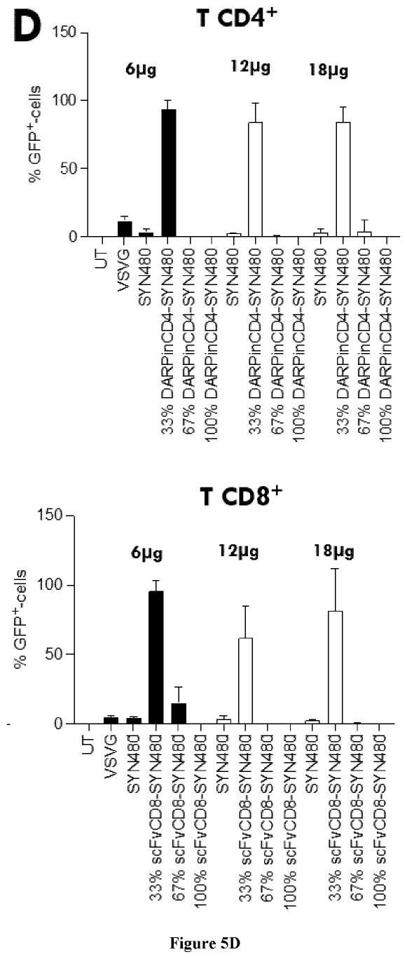

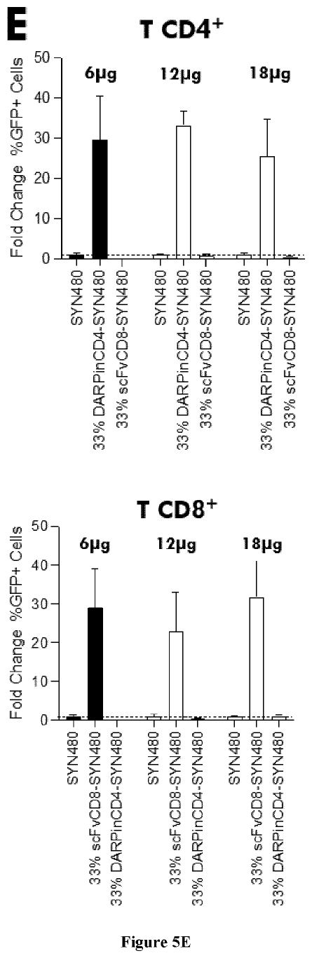

To this aim, we developed fusion protein containing either a DARPin against the CD4 receptor or an scFv against the CD8 receptor to target CD4⁺ and CD8⁺ T cells, respectively, using the previously described strategy (FIG. 5A). We collected peripheral blood mononuclear cells (PBMC) from healthy individuals and purified CD4⁺ and CD8⁺ T cells (FIG. 5B). To confirm the previous results observed with CB HSPCs, we produced LV batches with different types of envelopes by using different amounts of envelope plasmids and different stoichiometric ratios between SYN480 and SYN480 fused to a ligand. SYN480 envelope showed very poor ability to transduce both CD4⁺ and CD8⁺ T cells, ranging typically from less than 1% to 8% using LV produced with 18 g and 6 μg of envelope plasmid, respectively (FIGS. 5C and 5D). Surprisingly, addition of a ligand strongly increased the transduction efficiency using either 33% of DARPinCD4-SYN480 or 33% of scFvCD8-SYN480 envelope to transduce CD4⁺ or CD8⁺ T cells, respectively (FIGS. 5C and 5D). We observed between 80±10% up to 95±7% of GFP⁺ cells using 33% DARPinCD4-SYN480 LVs depending on the amount on plasmid using to produce LVs (FIGS. 5C and 5D). Overall, addition of a DARPin targeting CD4⁺ T cells and use of 33% of the DARPinCD4-SYN480 envelope plasmid allowed a significant increase (at least 25-fold change) in the average transduction efficiency in CD4⁺ T cells compared to SYN480 LV (FIG. 5E). Noteworthy, we observed very low transduction levels or no transduction using 67% or 100% of DARPinCD4-SYN480 ratios, confirming results obtained in CB HSPCs.

Similarly, fusion of a scFv targeting CD8⁺ enhanced transduction efficiency of CD8⁺ T cells (FIG. 5C). Using 33% scFvCD8-SYN480 LVs, we were able to transduce CD8⁺ T cells with an efficiency ranging from 62% using 12 μg of envelope plasmid to 95% using 6 μg of envelope plasmid (FIG. 5D). Thus, regardless of the amount of envelope used to pseudotype LVs, the 33% ratio of scFvCD8-SYN480 envelope allowed efficient transduction of CD8⁺ T cells. We observed at least a 22-fold increase in the percentage of GFP⁺ cells compared to SYN480 LV (FIG. 5E). The use of 67% and 100% of scFvCD8-SYN480 did not result in elevated levels of transduction (FIGS. 5C and 5D). Importantly, the use of the 33% DARPinCD4-SYN480 LVs and 33% scFvCD8-SYN480 LVs did not result in transduction of CD8⁺ T and CD4⁺ T cells, confirming the specificity of the engineered envelopes for the cell harboring the targeted cell-surface receptor. Of note, both 67% and 100% ratio of DARPinCD4-SYN480 and scFvCD8-SYN480 were failed to transduce CD8⁺ T and CD4⁺ T, respectively (data not shown).

LVs Targeting Human GLP1⁺ or IA2⁺ Cells

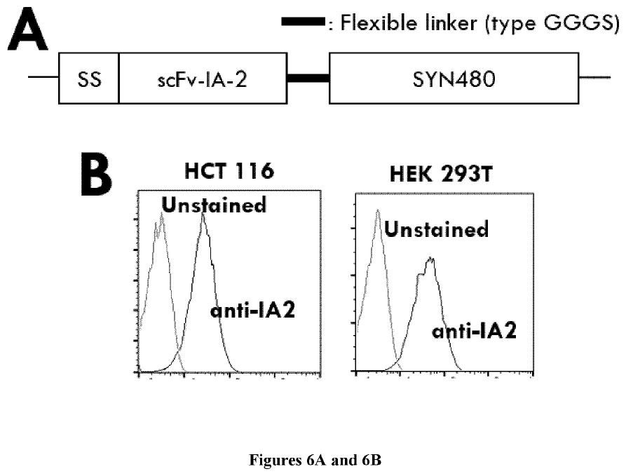

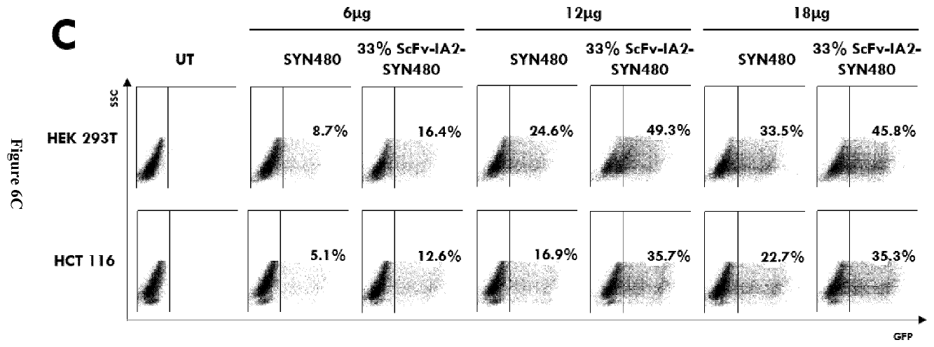

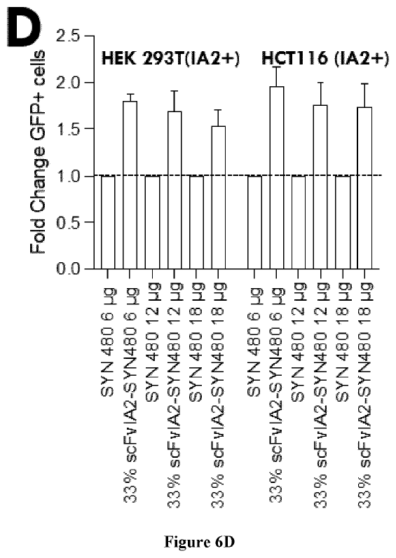

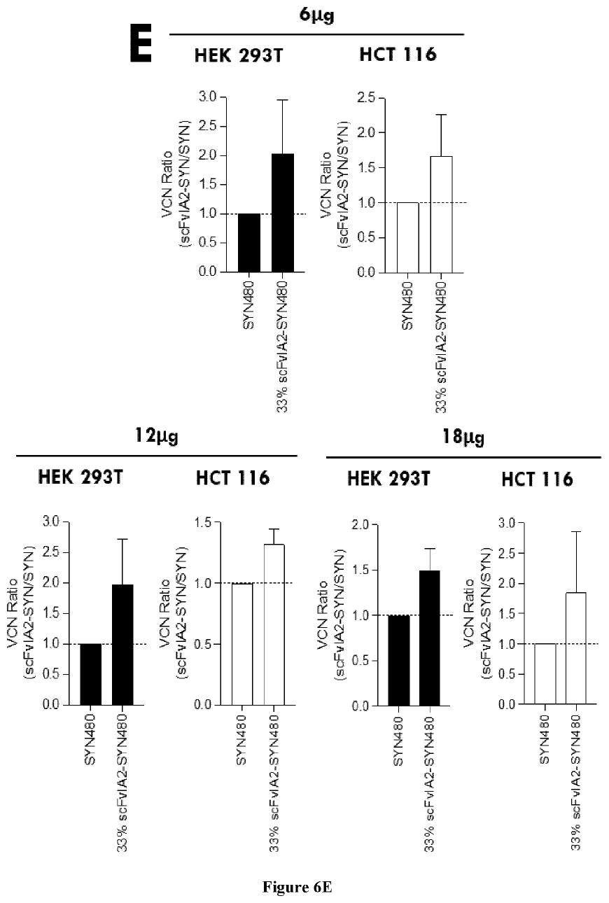

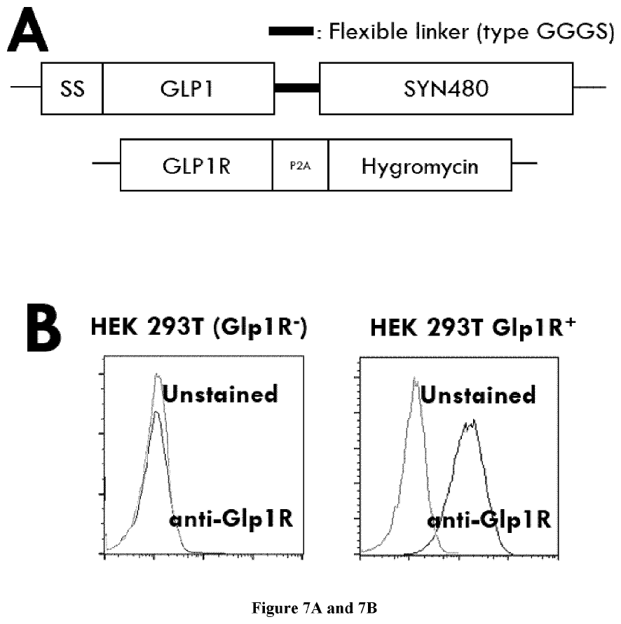

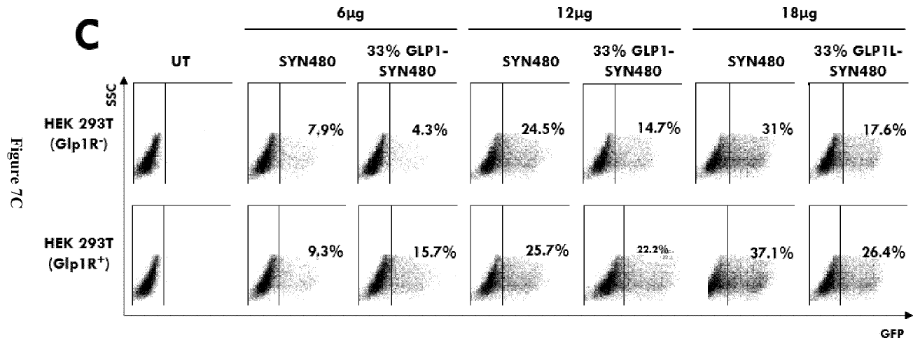

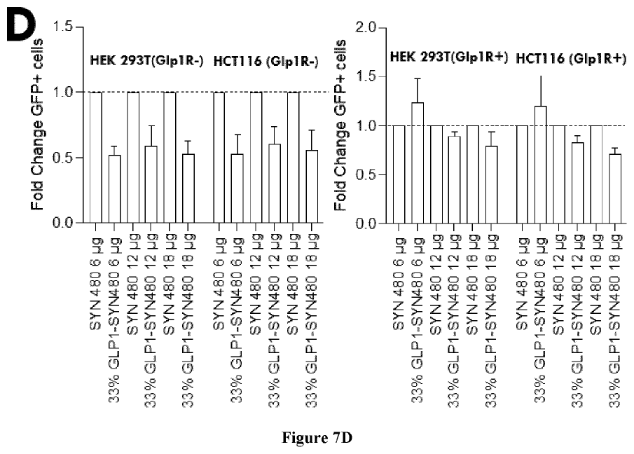

Lastly, we designed fusion proteins containing either the glucagon ligand (GLP1) or an scFv targeting IA2, a well-known receptor overexpressed on pancreatic cells from patients with type 1 diabetes; autoantibodies against IA2 are found in the vast majority of these patients^{12,13} (FIG. 6A & FIG. 7A). HEK 293T cells and HCT 116 cells naturally expressed IA2 on their surface as shown by flow cytometry analysis (FIG. 6B). Based on our previous results, we decided to test only the 33% scFvIA2-SYN480 ratio with the different amounts of envelope plasmids. Fusion of scFvIA2 consistently increased transduction (compared to SYN480 alone) in both HEK 293T cells and HCT 116 cells by at least 50% and up to 95%, depending of the envelope plasmid amount used for LV production (FIGS. 6C and 6D). As observed with CB HSPCs, the higher amount of envelope plasmid was used, the higher was the percentage of GFP⁺ cells, despite the decrease in the viral titer (Table 1). We also evaluated transduction efficiency by analyzing proviral integration one week after transduction. We observed an increased VCN using 33% scFvIA2-SYN480 LVs compared to SYN480 LVs in both HEK 293T cells and HCT 116 cells, confirming the results observed by flow cytometry (FIG. 6E). Therefore, we were again able to modify SYN's tropism using an alternative ligand/receptor combination on a new cell type.

In order to target pancreatic cells using an alternative receptor, we developed a fusion protein containing GLP1 between the SS and SYN480 sequences (FIG. 7A). HEK 293T cells and HCT 116 cells do not naturally express the Glp1R receptor on their surface, thus we stably transduced them with a construct to overexpress both Glp1R and hygromycin as a selection marker (FIG. 7B). Following the same strategy used with IA2 receptor, we transduced both Glp1R⁺ and Glp1R⁻ cells with either SYN480 or 33% GLP1-SYN480 LVs. We observed a reduced transduction efficiency of HEK 293T cells and HCT 116 cells (Glp1R) with 33% GLP1-SYN480 LVs compared to SYN480 LV, regardless of the different amount used to produce LVs (FIGS. 7C and 7D). We did not observe gain in transduction efficiency in Glp1R⁺ using 33% GLP1-SYN480 LVs compared to the SYN480 LVs, but rather a rescue of the reduced transduction efficiency observed in Glp1R⁻ cells (FIGS. 7C and 7D)

Overall, we developed a fusion strategy that allows modification of SYN's tropism towards different receptors in order to target the desired cell type. We demonstrated that we were able to transduce several different cell types using an appropriate ligand to target them. As shown, our system is adaptable to multiple desired antigens to retarget syncytin to a cell type of interest.

### REFERENCES

Throughout this application, various references describe the state of the art to which this invention pertains. The disclosures of these references are hereby incorporated by reference into the present disclosure.
1. Urlaub, G. & Chasin, L. A. Isolation of Chinese hamster cell mutants deficient in dihydrofolate reductase activity. Proc Natl Acad Sci USA 77, 4216-4220 (1980).
2. Mangeney, M. et al. Placental syncytins: Genetic disjunction between the fusogenic and immunosuppressive activity of retroviral envelope proteins. Proc Natl Acad Sci USA 104, 20534-20539 (2007).
3. Swaminathan, S. K. et al. Identification and characterization of a novel scFv recognizing human and mouse CD133. Drug Deliv Transl Res 3, 143-151 (2013).
4. Kolm-Litty, V. et al. Human monoclonal antibodies isolated from type I diabetes patients define multiple epitopes in the protein tyrosine phosphatase-like IA-2 antigen. J Immunol 165, 4676-4684 (2000).
5. Grandi, N. & Tramontano, E. HERV Envelope Proteins: Physiological Role and Pathogenic Potential in Cancer and Autoimmunity. Front Microbiol 9, 462 (2018).
6. Verhoeyen, E. et al. Stem cell factor-displaying simian immunodeficiency viral vectors together with a low conditioning regimen allow for long-term engraftment of gene-marked autologous hematopoietic stem cells in macaques. Hum Gene Ther 23, 754-768 (2012).
7. Drewlo, S., Leyting, S., Kokozidou, M., Mallet, F. & Pötgens, A. J. G. C-Terminal truncations of syncytin-1 (ERVWE1 envelope) that increase its fusogenicity. Biol Chem 387, 1113-1120 (2006).
8. Chang, C., Chen, P.-T., Chang, G.-D., Huang, C.-J. & Chen, H. Functional characterization of the placental fusogenic membrane protein syncytin. Biol Reprod 71, 1956-1962 (2004).
9. Coquin, Y., Ferrand, M., Seye, A., Menu, L. & Galy, A. Syncytins enable novel possibilities to transduce human or mouse primary B cells and to achieve well-tolerated in vivo gene transfer. http://biorxiv.org/lookup/doi/10.1101/816223 (2019) doi:10.1101/816223.
10. Logan, A. C. et al. Factors influencing the titer and infectivity of lentiviral vectors. Hum Gene Ther 15, 976-988 (2004).
11. Corre, G. et al. Lentiviral standards to determine the sensitivity of assays that quantify lentiviral vector copy numbers and genomic insertion sites in cells. Gene Ther 29, 536-543 (2022).
12. Aanstoot, H. J. et al. Identification and characterization of glima 38, a glycosylated islet cell membrane antigen, which together with GAD65 and IA2 marks the early phases of autoimmune response in type 1 diabetes. J Clin Invest 97, 2772-2783 (1996).
13. Roep, B. O. & Peakman, M. Antigen targets of type 1 diabetes autoimmunity. Cold Spring Harb Perspect Med 2, a007781 (2012).

1. A fusion protein wherein a syncytin-1 polypeptide is fused to one or more targeting-moieties, wherein the syncytin-1 polypeptide comprises the amino acid sequence as set forth in SEQ ID NO:2 (SDGGGXXDXXR) and is capable of binding to ASCT1 receptor and/or to ASCT2 receptor.

2. The fusion protein of claim 1 wherein the syncytin-1 polypeptide comprises the amino acid sequence as set forth in SEQ ID NO:3 (SDGGGVQDQAR).

3. The fusion protein of claim 2 wherein the syncytin-1 polypeptide comprises the amino acid sequence as set forth in SEQ ID NO:3 (SDGGGVQDQAR) and further comprises at least 15, 20, 25, 30, 35, 40, 45, 50, 100, 150, 200, 250, 300, 350, 400, or 450 consecutive amino acids of SEQ ID NO:1.

4. The fusion protein of claim 2 wherein the syncintin-1 polypeptide comprises an amino acid sequence having at 70% identity with the amino acid sequence that ranges from the amino acid residue at position 21 to the amino acid residue at position 480 in SEQ ID NO:1, or a variant thereof wherein the arginine residue (R) at position 393 is substituted by a glutamine residue (Q) and the phenylalanine residue (F) are position 399 is substituted by an alanine residue (A).

5. (canceled)

6. The fusion protein according to claim 1, wherein the one or more targeting-moieties is selected from the group consisting of ligands, antibodies, antibody fragments; non-antibody-based recognition scaffolds; anticalins; designed ankyrin repeat domains (DARPins); binding sites of a cysteine-rich polypeptide avimers; and afflins, and is suitable for targeting a population of cells selected from the group consisting of immune cells, hematopoietic cells, and malignant cells.

7. (canceled)

8. The fusion protein according to claim 1, wherein the one or more targeting-moieties has binding affinity for a CD (cluster of differentiation) molecule selected from the group consisting of CD1a, CD1b, CD1c, CD1d, CD1e, CD2, CD3delta, CD3epsilon, CD3gamma, CD4, CD5, CD6, CD7, CD8alpha, CD8beta, CD9, CD10, CD11a, CD11b, CD11c, CDw12, CD13, CD14, CD15u, CD16a, CD16b, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD44R, CD45, CD46, CD47R, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD65s, CD66a, CD66b, CD66c, CD66d, CD66e, CD66f, CD68, CD69, CD70, CD71, CD72, CD73, CD74, CD75, CD75s, CD77, CD79a, CD79b, CD80, CD81, CD82, CD83, CD84, CD85, CD86, CD87, CD88, CD89, CD90, CD91, CD92, CDw93, CD94, CD95, CD96, CD97, CD98, CD99, CD100, CD101, CD102, CD103, CD104, CD105, CD106, CD107a, CD107b, CD108, CD109, CD110, CD111, CD112, CDw113, CD114, CD115, CD116, CD117, CD118, CDw119, CD120a, CD120b, CD121a, CDw121b, CD122, CD123, CD124, CDw125, CD126, CD127, CDw128a, CDw128b, CD129, CD130, CD131, CD132, CD133, CD134, CD135, CDw136, CDw137, CD138, CD139, CD140a, CD140b, CD141, CD142, CD143, CD144, CDw145, CD146, CD147, CD148, CDw149, CD150, CD151, CD152, CD153, CD154, CD155, CD156a, CD156b, CDw156C, CD157, CD158, CD159a, CD159c, CD160, CD161, CD162, CD162R, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CDw186, CD191, CD192, CD193, CD195, CD196, CD197, CDw198, CDw199, CDw197, CD200, CD201, CD202b, CD203c, CD204, CD205, CD206, CD207, CD208, CD209, CDw210, CD212, CD213a1, CD213a2, CDw217, CDw218a, CDw218b, CD220, CD221, CD222, CD223, CD224, CD225, CD226, CD227, CD228, CD229, CD230, CD231, CD232, CD233, CD234, CD235a, CD235b, CD235ab, CD236, CD236R, CD238, CD239, CD240CE, CD240D, CD240DCE, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD271, CD272, CD273, CD274, CD275, CD276, CD277, CD278, CD279, CD280, CD281, CD282, CD283, CD284, CD289, CD292, CDw293, CD294, CD295, CD296, CD297, CD298, CD299, CD300a, CD300c, CD300e, CD301, CD302, CD303, CD304, CD305, CD306, CD307, CD309, CD312, CD314, CD315, CD316, CD317, CD318, CD319, CD320, CD321, CD322, CD324, CDw325, CD326, CDw327, CDw328, CDw329, CD331, CD332, CD333, CD334, CD335, CD336, CD337, CDw338, and CD339.

9. The fusion protein according to claim 1, wherein the one or more targeting-moieties has biding affinity for a cell surface molecule selected from the group consisting of 2B4/CD244/SLAMF4, ABCG2, Aldehyde Dehydrogenase 1-A1/ALDH1A1, BMI-1, C1qR1/CD93, CD34, CD38, CD44, CD45, CD48/SLAMF2, CD90/Thy1, CD117/c-kit, CD133, CDCP1, CXCR4, Endoglin/CD105, EPCR, Erythropoietin R, ESAM, EVI-1, Integrin alpha 6/CD49f, SLAM/CD150, VCAM-1/CD 106 and VEGFR2/KDR/Flk-1

10. The fusion protein according to claim 1, wherein the one or more targeting-moieties is the Stem Cell Factor (SCF), which binds CD117 (c-kit) receptor comprising an amino acid sequence having at least 70% of identity with the amino acid sequence as set forth in SEQ ID NO:4; a single-chain fragment variant (scFv) directed against CD 133 receptor comprising an amino acid sequence having at least 70% of identity with the amino acid sequence as set forth in SEQ ID NO:5; a DARPin directed against CD4 comprising an amino acid sequence having at least 70% of identity with the amino acid sequence as set forth in SEQ ID NO:6; a single-chain fragment variant (scFv) directed against CD8 comprising an amino acid sequence having at least 70% of identity with the amino acid sequence as set forth in SEQ ID NO:7; a single-chain fragment variant (scFv) directed against IA-2 receptor comprising an amino acid sequence having at least 70% of identity with the amino acid sequence as set forth in SEQ ID NO:8; or GLP1 comprising an amino acid sequence having at least 70% of identity with the amino acid sequence as set forth in SEQ ID NO:9.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. The fusion protein according to claim 1, wherein a C-terminal end of the one or more targeting-moieties is fused to an N-terminal end of the sycintin-1 polypeptide.

23. The fusion protein according to claim 1, wherein the syncytin-1 polypeptide and the one or more targeting-moieties are fused to each other directly or via a linker.

24. The fusion protein according to claim 1 that further comprises the sequence of a signal peptide.

25. The fusion protein according to claim 1 comprising the amino acid sequence as set forth in SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO:19, SEQ ID NO:20 or SEQ ID NO:21.

26. (canceled)

27. A polynucleotide that encodes for the fusion protein according to claim 1, a vector comprising the polynucleotide or a host cell which has been transfected, infected or transformed by the polynucleotide and/or the vector.

28. (canceled)

29. (canceled)

30. A virus particle functionalized with or a virus-like particle pseudotyped with the fusion protein according to claim 1 and comprising one or more viral protein(s) and one more cargo(s); a cell line for producing the virus particle or the virus-like particle; or a pharmaceutical composition comprising a plurality of the virus particle or the virus-like particle.

31. (canceled)

32. The virus particle or the virus-like particle of claim 30 that comprises a Gag protein.

33. The virus particle or the virus-like particle of claim 30, wherein the one or more viral protein(s) has an amino acid sequence having at least 70% identity with the amino acid sequence a set forth in SEQ ID NO:22 or SEQ ID NO:23.

34. The virus particle or the virus-like particle according to claim 30, wherein the one more cargo(s) is selected from the group consisting of organic molecules, polymers, polypeptides, polynucleotides and small organic compounds having a molecular weight of more than 50 and less than about 2,500 daltons.

35. The virus particle or the virus-like particle of claim 34 wherein the one more cargo(s) is a polynucleotide.

36. (canceled)

37. The virus particle or the virus-like particle according to claim 30, wherein the one more cargo(s) is a polypeptide and the one or more viral protein(s) is a viral structural protein, and wherein the polypeptide is fused to the viral structural protein either directly or via a linker.

38. (canceled)

39. A cell line for producing the virus particle or the virus-like particle according to claim 30, comprising i) one or more polynucleotides encoding the structural viral proteins required for forming the said the virus particle, ii) a polynucleotide comprising an amino acid sequence having at least 70% of identity with the amino acid sequence as set forth in SEQ ID NO:7, and iii) one or more polynucleotide encoding for the cargo(s).

40. (canceled)

41. (canceled)

42. (canceled)


SEQUENCE LISTING (41 sequences)

SEQ ID NO: 1 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 2 - protein, 11 aa, Homo sapiens
  VARIANT 6: note = X represents any amino acid
  VARIANT 7: note = X represents any amino acid
  VARIANT 9: note = X represents any amino acid
  VARIANT 10: note = X represents any amino acid
SDGGGXXDXXR

SEQ ID NO: 3 - protein, 11 aa, Homo sapiens
SDGGGVQDQAR

SEQ ID NO: 4 - protein, 212 aa, Homo sapiens
CRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKR

SEQ ID NO: 5 - protein, 243 aa, synthetic construct
MDIVLSQSPAIMSASPGEKVTISCSASSSVSYMYWYQQKPGSSPKPWIYRTSNLASGVPARFSGSGSGTSYSLTISSMEAEDAATYYCQQYHSYPPTFGAGTKLELKSSGGGGSGGGGGGSSRSSLEVKLVESGPELKKPGETVKISCKASGYTFTDYSMHWVNQAPGKGLKWMGWINTETGEPSYADDFKGRFAFSLETSASTAYLQINNLKNEDTATYFCATDYGDYFDYWGQGTTLTVSS

SEQ ID NO: 6 - protein, 129 aa, synthetic construct
AAQPADLGKKLLEAARAGQDDEVRILMANGADVNATDTLGRTPLHLAAQNGHLEIVEVLLKHSADVNAIEEVGMTPLHLAVVAGHLEIVEVLLKNGADVNAQDKFGKTAFDISIDYGNEDLAEILQKLN

SEQ ID NO: 7 - protein, 246 aa, synthetic construct
AAQPAQVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSGGGGSGGGGSGGGGSDIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIKR

SEQ ID NO: 8 - protein, 245 aa, synthetic construct
QVQLQESGPGLVKPSETLSLRCNVSGVSISGFYWGWIRQPPGKGLEWIGHIFSSGSTDYSPSLKSRVDVSMDTSKNHFSLNLSSVTAADTAVYYCARGLKGVATASFDFWGRGTLVTVSSSSGGGGSGGGGSGGGGSYVLTQPPSVSVAPGKTATITCGADNIGTKSVHWYQQRPGQAPMLVIYYNKNRPSGIPERFSGSNSGHTATLTISRVEAGDEAAYYCQVWDTRSDLVVFGGGTKLTVLG

SEQ ID NO: 9 - protein, 31 aa, synthetic construct
HAEGTFTSDVSSYLEGQAAKEFIAWLVKGRG

SEQ ID NO: 10 - protein, 725 aa, synthetic construct
MALPYHIFLFTVLLPSFTLTYPYDVPDYAEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAAGGGSGGGGSGGGSAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAEQGGTCLALGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 11 - protein, 803 aa, synthetic construct
MALPYHIFLFTVLLPSFTLTYPYDVPDYAMDIVLSQSPAIMSASPGEKVTISCSASSSVSYMYWYQQKPGSSPKPWIYRTSNLASGVPARFSGSGSGTSYSLTISSMEAEDAATYYCQQYHSYPPTFGAGTKLELKSSGGGGSGGGGGGSSRSSLEVKLVESGPELKKPGETVKISCKASGYTFTDYSMHWVNQAPGKGLKWMGWINTETGEPSYADDFKGRFAFSLETSASTAYLQINNLKNEDTATYFCATDYGDYFDYWGQGTTLTVSSGGGSGGGGSGGGSAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAEQGGTCLALGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 12 - protein, 689 aa, synthetic construct
MALPYHIFLFTVLLPSFTLTYPYDVPDYAAAQPADLGKKLLEAARAGQDDEVRILMANGADVNATDTLGRTPLHLAAQNGHLEIVEVLLKHSADVNAIEEVGMTPLHLAVVAGHLEIVEVLLKNGADVNAQDKFGKTAFDISIDYGNEDLAEILQKLNGGGSGGGGSGGGSAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAEQGGTCLALGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 13 - protein, 806 aa, synthetic construct
MALPYHIFLFTVLLPSFTLTYPYDVPDYAAAQPAQVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSGGGGSGGGGSGGGGSDIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIKRGGGSGGGGSGGGSAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAEQGGTCLALGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 14 - protein, 805 aa, synthetic construct
MALPYHIFLFTVLLPSFTLTYPYDVPDYAQVQLQESGPGLVKPSETLSLRCNVSGVSISGFYWGWIRQPPGKGLEWIGHIFSSGSTDYSPSLKSRVDVSMDTSKNHFSLNLSSVTAADTAVYYCARGLKGVATASFDFWGRGTLVTVSSSSGGGGSGGGGSGGGGSYVLTQPPSVSVAPGKTATITCGADNIGTKSVHWYQQRPGQAPMLVIYYNKNRPSGIPERFSGSNSGHTATLTISRVEAGDEAAYYCQVWDTRSDLVVFGGGTKLTVLGGGGSGGGGSGGGSAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAEQGGTCLALGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 15 - protein, 591 aa, synthetic construct
MALPYHIFLFTVLLPSFTLTYPYDVPDYAHAEGTFTSDVSSYLEGQAAKEFIAWLVKGRGGGGSGGGGSGGGSAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAEQGGTCLALGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 16 - protein, 667 aa, synthetic construct
MALPYHIFLFTVLLPSFTLTYPYDVPDYAEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAAGGGSGGGGSGGGSAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAEQGGTCLALGEECCYYVNQSGIVTEKVKEIRDRIQRRAEELRNTGPWGLLSQWMPWILPFLGPLAAIILLLLFGPCIFNLLVNFVSSRI

SEQ ID NO: 17 - protein, 745 aa, synthetic construct
MALPYHIFLFTVLLPSFTLTYPYDVPDYAMDIVLSQSPAIMSASPGEKVTISCSASSSVSYMYWYQQKPGSSPKPWIYRTSNLASGVPARFSGSGSGTSYSLTISSMEAEDAATYYCQQYHSYPPTFGAGTKLELKSSGGGGSGGGGGGSSRSSLEVKLVESGPELKKPGETVKISCKASGYTFTDYSMHWVNQAPGKGLKWMGWINTETGEPSYADDFKGRFAFSLETSASTAYLQINNLKNEDTATYFCATDYGDYFDYWGQGTTLTVSSGGGSGGGGSGGGSAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAEQGGTCLALGEECCYYVNQSGIVTEKVKEIRDRIQRRAEELRNTGPWGLLSQWMPWILPFLGPLAAIILLLLFGPCIFNLLVNFVSSRI

SEQ ID NO: 18 - protein, 631 aa, synthetic construct
MALPYHIFLFTVLLPSFTLTYPYDVPDYAAAQPADLGKKLLEAARAGQDDEVRILMANGADVNATDTLGRTPLHLAAQNGHLEIVEVLLKHSADVNAIEEVGMTPLHLAVVAGHLEIVEVLLKNGADVNAQDKFGKTAFDISIDYGNEDLAEILQKLNGGGSGGGGSGGGSAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAEQGGTCLALGEECCYYVNQSGIVTEKVKEIRDRIQRRAEELRNTGPWGLLSQWMPWILPFLGPLAAIILLLLFGPCIFNLLVNFVSSRI

SEQ ID NO: 19 - protein, 748 aa, synthetic construct
MALPYHIFLFTVLLPSFTLTYPYDVPDYAAAQPAQVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSGGGGSGGGGSGGGGSDIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIKRGGGSGGGGSGGGSAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAEQGGTCLALGEECCYYVNQSGIVTEKVKEIRDRIQRRAEELRNTGPWGLLSQWMPWILPFLGPLAAIILLLLFGPCIFNLLVNFVSSRI

SEQ ID NO: 20 - protein, 747 aa, synthetic construct
MALPYHIFLFTVLLPSFTLTYPYDVPDYAQVQLQESGPGLVKPSETLSLRCNVSGVSISGFYWGWIRQPPGKGLEWIGHIFSSGSTDYSPSLKSRVDVSMDTSKNHFSLNLSSVTAADTAVYYCARGLKGVATASFDFWGRGTLVTVSSSSGGGGSGGGGSGGGGSYVLTQPPSVSVAPGKTATITCGADNIGTKSVHWYQQRPGQAPMLVIYYNKNRPSGIPERFSGSNSGHTATLTISRVEAGDEAAYYCQVWDTRSDLVVFGGGTKLTVLGGGGSGGGGSGGGSAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAEQGGTCLALGEECCYYVNQSGIVTEKVKEIRDRIQRRAEELRNTGPWGLLSQWMPWILPFLGPLAAIILLLLFGPCIFNLLVNFVSSRI

SEQ ID NO: 21 - protein, 533 aa, synthetic construct
MALPYHIFLFTVLLPSFTLTYPYDVPDYAHAEGTFTSDVSSYLEGQAAKEFIAWLVKGRGGGGSGGGGSGGGSAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAEQGGTCLALGEECCYYVNQSGIVTEKVKEIRDRIQRRAEELRNTGPWGLLSQWMPWILPFLGPLAAIILLLLFGPCIFNLLVNFVSSRI

SEQ ID NO: 22 - protein, 708 aa, Homo sapiens
MTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGKLPGPAVEGPSATGPEIIRSPQDDASSPHLQVMLQIHLPGRHTLFVRAMIDSGASGNFIDHEYVAQNGIPLRIKDWPILVEAIDGRPIASGPVVHETHDLIVDLGDHREVLSFDVTQSPFFPVVLGVRWLSTHDPNITWSTRSIVFDSEYCRYHCRMYSPIPPSLPPPAPQPPLYYPVDGYRVYQPVRYYYVQNVYTPVDEHVYPDHRLVDPHIEMIPGAHSIPSGHVYSLSEPEMAALRDFVARNVKDGLITPTIAPNGAQVLQVKRGWKLQVSYDCRAPNNFTIQNQYPRLSIPNLEDQAHLATYTEFVPQIPGYQTYPTYAAYPTYPVGFAWYPVGRDGQGRSLYVPVMITWNPHWYRQPPVPQYPPPQPPPPPPPPPPPPSYSTL

SEQ ID NO: 23 - protein, 325 aa, Homo sapiens
MTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGNSPAPL

SEQ ID NO: 24 - DNA, 2419 bp, synthetic construct
  source 1..2419: mol_type = other DNA
gactatggccctcccttatcatatttttctctttactgttcttttaccctctttcactctcacttatccatatgatgttccagattatgctatggacattgttctctcccagtctccagcaatcatgtctgcatctccaggggagaaggtcaccatatcctgcagtgccagctcaagtgtaagttatatgtactggtaccagcagaagccaggatcctcccccaaaccctggatttatcgcacatccaacctggcttctggagtccctgctcgcttcagtggcagtgggtctgggacctcttactctctcacaatcagcagcatggaggctgaagatgctgccacttattactgccagcagtatcatagttacccacccacgttcggtgctgggaccaagctggagctgaaatcctctggtggcggtggctcgggcggtggtgggggtggttcctctagatcttccctggaagtgaagctggtggagtctggacctgagctgaagaagcctggagagacagtcaagatctcctgcaaggcttctggttataccttcacagactattcaatgcactgggtgaatcaggctccaggaaagggtttaaagtggatgggctggataaacactgagactggtgagccatcatatgcagatgacttcaagggacggtttgccttctctttggaaacctctgccagcactgcctatttgcagatcaacaacctcaaaaatgaggacacggctacatatttctgtgctaccgattacggggactactttgactactggggccaaggcaccactctcacagtctcctcaggcggtggctctggaggtggtgggtccggaggtggctctgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaacaagggggaacctgtttagccttaggggaagaatgctgttattatgttaatcaatctggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttagtga

SEQ ID NO: 25 - DNA, 2185 bp, synthetic construct
  source 1..2185: mol_type = other DNA
gactatggccctcccttatcatatttttctctttactgttcttttaccctctttcactctcacttatccatatgatgttccagattatgctgaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagattccagagtcagtgtcacaaaaccatttatgttaccccctgttgcagccggcggtggctctggaggtggtgggtccggaggtggctctgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaacaagggggaacctgtttagccttaggggaagaatgctgttattatgttaatcaatctggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttagtga

SEQ ID NO: 26 - DNA, 1903 bp, synthetic construct
  source 1..1903: mol_type = other DNA
gactatggccctcccttatcatatttttctctttactgttcttttaccctctttcactctcacttatccatatgatgttccagattatgctgcggcccagccggccgacctgggtaagaaactgctggaagctgctcgtgctggtcaggacgacgaagttcgtatcctgatggctaacggagcagatgtcaacgctactgacactcttggtcgtactccgctacacctggctgctcagaatggacatctcgaaatcgtagaagttctcctgaagcacagtgctgacgttaacgctattgaagaggttggtatgactccgctgcacctggctgttgttgctggtcacctggaaatcgttgaagttctgctgaagaacggtgctgacgttaacgctcaggacaaattcggtaagaccgctttcgacatctccatcgactacggtaacgaggacctggctgaaatcctgcaaaagcttaatggcggtggctctggaggtggtgggtccggaggtggctctgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaacaagggggaacctgtttagccttaggggaagaatgctgttattatgttaatcaatctggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatctagtga

SEQ ID NO: 27 - DNA, 2296 bp, synthetic construct
  source 1..2296: mol_type = other DNA
gactatggccctcccttatcatatttttctctttactgttcttttaccctctttcactctcacttatccatatgatgttccagattatgctggaggtggctctggtggaggaggctctggaggtggtggatcagcggcccagccggcccaggtgcagctggtgcagagcggagccgaggataagaaacctggcgctagcgtgaaggtgtcctgcaaggccagcggcttcaacatcaaggacacctacatccactgggtgcgccaggctccaggacagggcctggaatggatgggccggatcgaccccgccaacgacaacaccctgtacgccagcaagttccagggcagagtgaccatcaccgccgacaccagcagcaacaccgcctacatggaactgagcagcctgagaagcgaggacaccgccgtgtactactgtggcagaggctacggctactacgtgttcgaccactggggccagggcaccaccgtgacagtgtctagcggcggaggcggatctggaggcggaggatcaggaggcggcggatccgacatcgtgatgacccagagccctagcagcctgtctgccagcgtgggcgaccgcgtgaccattacctgccggaccagccggtccatcagccagtacctggcctggtatcaggaaaagcccggcaaggcccccaagctgctgatctacagcggcagcaccctgcagagcggcgtgccaagcagattcagcggcagcggctccggcaccgacttcaccctgaccatcagcagcctgcagcccgaggacttcgccacctactactgccagcagcacaacgagaaccccctgaccttcgggcagggaacaaaggtggagatcaagagaggcggtggttcaggtggaggtgggtccggaggtggctctgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaacaagggggaacctgtttagccttaggggaagaatgctgttattatgttaatcaatctggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatctagtga

SEQ ID NO: 28 - DNA, 2250 bp, synthetic construct
  source 1..2250: mol_type = other DNA
gactatggccctcccttatcatatttttctctttactgttcttttaccctctttcactctcacttatccatatgatgttccagattatgctcaggtccagctgcaagagtctggccctggcctggtcaagcctagcgaaacactgagcctgcggtgcaatgtgtccggcgtgtccatcagcggcttttactggggctggatcagacagcctcctggcaaaggcctggaatggatcggccacatctttagcagcggcagcaccgactatagccccagcctgaagtctagagtggacgtgtccatggataccagcaagaaccacttcagcctgaacctgagcagcgtgacagccgccgatacagccgtgtactattgtgccagaggcctgaaaggcgtggcaaccgccagcttcgatttttggggcagaggcaccctggtcaccgtgtctagttctagcggaggcggaggatctggtggcggaggaagtggcggaggcggttcttatgttctgacccagcctcctagcgtgtcagtggcccctggaaagaccgccacaattacctgtggcgccgacaacatcggcaccaagagcgtgcactggtatcagcagaggccaggacaggcccctatgctggtcatctactacaacaagaacagacccagcggcatccccgagagattcagcggctctaatagcggccacaccgccacactgaccatctccagagttgaagccggcgacgaggccgcctactactgtcaagtttgggatacccgcagcgacctggtggtttttggcggcggaacaaagctgacagtgctgggaggcggtggctctggaggtggtgggtccggaggtggctctgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaacaagggggaacctgtttagccttaggggaagaatgctgttattatgttaatcaatctggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatctagtg

SEQ ID NO: 29 - DNA, 1609 bp, synthetic construct
  source 1..1609: mol_type = other DNA
gactatggccctcccttatcatatttttctctttactgttcttttaccctctttcactctcacttatccatatgatgttccagattatgctcacgccgagggcaccttcaccagcgacgtgagcagctacctggagggccaggccgccaaggagttcatcgcttggctggttaagggtaggggaggcggtggctctggaggtggtgggtccggaggtggctctgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaacaagggggaacctgtttagccttaggggaagaatgctgttattatgttaatcaatctggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatctagtga

SEQ ID NO: 30 - DNA, 18 bp, synthetic construct
  source 1..18: mol_type = other DNA
tgggtccggaggtggctc

SEQ ID NO: 31 - DNA, 49 bp, synthetic construct
  source 1..49: mol_type = other DNA
cctaactcgaggacttgagtcattagattctggaagagacaaagttaac

SEQ ID NO: 32 - DNA, 88 bp, synthetic construct
  source 1..88: mol_type = other DNA
ttccttaagactatggccctcccttatcatatttttctctttactgttcttttaccctctttcactctcactgcaccccctccatgcc

SEQ ID NO: 33 - DNA, 49 bp, synthetic construct; identical to SEQ ID NO: 31
  source 1..49: mol_type = other DNA

SEQ ID NO: 34 - DNA, 2532 bp, synthetic construct
  source 1..2532: mol_type = other DNA
agattgattcaccggtcgccaccatggccggtgcaccaggacctctgcgacttgcgctgctgctgctcgggatggtcggacgagctggacctcgcccgcaaggtgcaacggtctctctctgggaaactgttcaaaaatggcgagagtacagacggcagtgccagagatccttgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccgcttctgcacagctgaaggcctctggctgcagaaggacaactccagcctgccctggagggacttgtcggagtgcgaggagtccaagcgaggggaaagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttatctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttgccattggggtgaacttcctcatctttgttcgggtcatctgcatcgtggtatccaaactgaaggccaatctcatgtgcaagacagacatcaaatgcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatattatactgctttgtcaacaatgaggtccagctggaatttcggaagagctgggagcgctggcggcttgagcacttgcacatccagagggacagcagcatgaagcccctcaagtgtcccaccagcagcctgagcagtggagccacggcgggcagcagcatgtacacagccacttgccaggcctcctgcagcggatccggcgcaacaaacttctctctgctgaaacaagccggagatgtcgaagagaatcctggaccgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcggccgcgtcgacaatcaacctctgga

SEQ ID NO: 35 - DNA, 22 bp, synthetic construct
  source 1..22: mol_type = other DNA
gctgtcatctcttgtgggctgt

SEQ ID NO: 36 - DNA, 21 bp, synthetic construct
  source 1..21: mol_type = other DNA
actcatgggagctgctggttc

SEQ ID NO: 37 - DNA, 26 bp, synthetic construct
  source 1..26: mol_type = other DNA
cctgtcatgcccacacaaatctctcc

SEQ ID NO: 38 - DNA, 20 bp, synthetic construct
  source 1..20: mol_type = other DNA
atggtgagcaagggcgagga

SEQ ID NO: 39 - DNA, 21 bp, synthetic construct
  source 1..21: mol_type = other DNA
cttcagggtcagcttgccgta

SEQ ID NO: 40 - DNA, 25 bp, synthetic construct
  source 1..25: mol_type = other DNA
aaacggccacaagttcagcgtgtcc

SEQ ID NO: 41 - protein, 9 aa, synthetic construct
YPYDVPDYA